(19) United States
(12) Patent Application Publication
(10) Pub. No.: US 2023/0114264 A1
(43) Pub. Date: Apr. 13, 2023

(54) ACTIVATION OF LYTIC GENES IN CANCER CELLS

(71) Applicants: The Jackson Laboratory, Bar Harbor, ME (US); The Chinese University of Hong Kong, Hong Kong Sar (CN)

(72) Inventors: Albert Cheng, Bar Harbor, ME (US); Kwok Wai Lo, Hong Kong Sar (CN); Pok Man Tom Hau, Hong Kong Sar (CN); Man Wu, Hong Kong Sar (CN)

(73) Assignees: The Jackson Laboratory, Bar Harbor, ME (US); The Chinese University of Hong Kong, Hong Kong Sar (CN)

(21) Appl. No.: 17/802,751

(22) PCT Filed: Feb. 26, 2021
(86) PCT No.: PCT/US2021/019880

§ 371 Date: Aug. 26, 2022

Related U.S. Application Data
(60) Provisional application No. 62/983,495, filed on Feb. 28, 2020.

(51) Int. Cl. C12N 15/63; C12N 15/11; C12N 9/22; C12N 15/85; A61K 31/522; A61K 38/46; A61P 35/00

(52) U.S. Cl. CPC C12N 15/635; C12N 15/11; C12N 9/22; C12N 15/85; A61K 31/522; A61K 38/465; A61P 35/00; C12N 2310/20; C12N 2800/80; C12N 2830/003; C12N 2800/107

(57) ABSTRACT

The present disclosure provides methods of inducing EBV early lytic cycle genes with high specificity. These methods slow or stop cancer cell growth in vitro and in vivo.

### RELATED APPLICATIONS

This application claims the benefit under 35 U.S.C. § 119(e) of U.S. provisional application No. 62/983,495, filed Feb. 28, 2020, which is incorporated by reference herein in its entirety.

### GOVERNMENT LICENSE RIGHTS

This invention was made with government support under 1R01HG009900 and P30CA03419 awarded by National Institutes of Health. The government has certain rights in the invention.

### BACKGROUND

Epstein-Barr virus (EBV) is etiologically linked to a remarkably wide range of human lymphoid malignancies (B-cell lymphoma (BL), Hodgkin's disease (HD), and other lymphomas) and two distinct types of epithelial cancers, gastric cancer (GC) and nasopharyngeal carcinoma (NPC). EBV-associated gastric cancers represent approximately 10% of all gastric cancers and are not an endemic disease. Among the 200,000 new cases of EBV-associated cancers reported annually worldwide, 84,000 and 78,000 are GC and NPC, respectively. In the endemic regions, including Hong Kong and South China, almost all NPCs belong to a non-keratinizing subtype that is consistently associated with EBV infection. EBV infection is also detected in 16% of conventional gastric adenocarcinomas and 89% of lympho-epithelioma-like gastric carcinomas. Lymphoeptithelioma-like carcinoma (LELC) is defined as a poorly differentiated carcinoma with dense lymphocytic infiltration and has similar histological features to undifferentiated NPC.

As a complex malignancy, EBV infection and a combination of multiple genetic aberrations contribute to NPC and GC tumorigenesis. These EBV-associated cancers are clonal malignancies derived from a single progenitor cell that was latently-infected with EBV. It is thought that genetic alterations in premalignant nasopharyngeal epithelium support a switch in cellular context to support persistent latent EBV infection. EBV infection and expression of latent viral genes e.g., EBNA1, LMP1, and LMP2A, and BART-microRNAs, then drive the clonal expansion of an infected epithelial cell during transformation. The clonal EBV genome and expression of viral transcripts in tumor cells strongly implicate EBV having critical roles in initiation and progression of NPC and GC.

### SUMMARY

The present disclosure provides, in some aspects, an efficient gene-editing method and associated tools for direct activation of EBV lytic genes. The technology provided herein overcomes the highly complex regulatory mechanism of lytic gene expression by taking advantage of the high copy number of EBV episomes in cancer cells, such as NPC cells. The artificial transcription factor system described herein utilizes a programmable DNA-binding protein to enable highly efficient lytic gene expression in EBV-positive cancer cells. Further, this forced activation of EBV-lytic gene transcription, including transcription of the EBV-encoded kinase BGLF4, enhances efficient conversion of the antiviral non-toxic prodrug form of ganciclovir to its cytotoxic DNA replication inhibitor form for cytolytic treatment.

In some aspects, the present disclosure is a method for activating an Epstein-Barr virus (EBV) gene, comprising introducing into a cell infected with EBV a programmable DNA binding protein system that targets a transcriptional regulatory sequence of a lytic EBV gene, and a transcriptional activator that is linked to a component of the programmable DNA binding protein system and is capable of activating transcription of the lytic EBV gene.

In some aspects, the present disclosure is a method comprising administering to a subject a programmable DNA binding protein system that targets a transcriptional regulatory sequence of a lytic EBV gene, and a transcriptional activator that is linked to a component of the programmable DNA binding protein system and is capable of activating transcription of the lytic EBV gene, wherein the subject has a cancer associated with EBV infection.

In some embodiments, the programmable DNA binding protein system includes a catalytically-inactive RNA-guided engineered nuclease (RGEN) or a nucleic acid encoding a catalytically-inactive RGEN, and a gRNA that targets the transcriptional regulatory sequence or a nucleic acid encoding a gRNA that targets the transcriptional regulatory sequence. In some embodiments, the gRNA binds to the transcriptional regulatory sequence.

In some embodiments, the gRNA is linked to a Pumilio-FBF (PUF) domain binding sequence (PBS). In some embodiments, the transcriptional activator is linked to a PUF domain that binds to the PBS of the gRNA. In some embodiments, the catalytically-inactive RGEN or the gRNA is linked to the transcriptional activator. In some embodiments, the catalytically-inactive RGEN is dCas9.

In some embodiments, the programmable DNA binding protein system includes a transcription activator-like effector (TALE) linked to the transcriptional activator. In some embodiments, the programmable DNA binding protein system includes a zinc finger protein (ZFP) linked to the transcriptional activator.

In some embodiments, the lytic EBV gene is an immediate-early viral transactivator. In some embodiments, the immediate-early viral transactivator is selected from BZLF1 and BRLF1.

In some embodiments, the lytic EBV gene is a protein kinase (PK) gene. In some embodiments, the PK gene is BGLF4. In some embodiments, the lytic EBV gene is a thymidine kinase gene. In some embodiments, the thymidine kinase gene is BXLF1. In some embodiments, the lytic EBV gene is essential for DNA polymerase activity. In some embodiments, the lytic EBV gene essential for DNA polymerase activity is BMRF1.

In some embodiments, the method further comprises introducing into the cell an antiviral agent. In some embodiments, the antiviral agent is a prodrug. In some embodiments, the prodrug is selected from ganciclovir, acyclovir, enciclovir, penciclovir, valacyclovir, famciclovir, and bromovinyldeoxyuridine. In some embodiments, the prodrug is ganciclovir.

In some embodiments, the transcriptional regulatory sequence is a promoter sequence. In some embodiments, the transcriptional activator binds to the transcriptional regulatory sequence. In some embodiments, the transcriptional activator comprises or encodes a heat shock factor 1 (HSF1) transactivation domain. In some embodiments, the transcriptional activator comprises or encodes p65HSF1.

In some embodiments, the expression of a component of the programmable DNA binding protein system is inducible. In some embodiments, expression of the transcriptional activator is inducible.

In some embodiments, the cell is a mammalian cell. In some embodiments, the mammalian cell is a cancer cell.

In some aspects, the present disclosure is a method of synergistic Epstein Barr virus (EBV) lytic activation, comprising introducing into a cell infected with EBV (a) a programmable DNA binding protein system that targets a transcriptional regulatory sequence of EBV BZLF1 and a transcriptional regulatory sequence of EBV BRLF1, and (b) a transcriptional activator that is linked to a component of the programmable DNA binding protein system and is capable of activating transcription of the EBV BZLF1 and EBV BRLF1, wherein expression of genes regulated by EBV BZLF1 and EBV BRLF1 is at least 2-fold higher than expression of the same genes resulting from introduction of a programmable DNA binding protein system that targets only EBV BZLF1 or only EBV BRLF1. In some embodiments, the genes regulated by EBV BZLF1 and EBV BRLF1 include EBV protein kinase (PK) and EBV early antigen diffuse component (EA-D) genes.

In some aspects, the present disclosure is a kit comprising a programmable DNA binding protein system that targets a transcriptional regulatory sequence of a lytic EBV gene, a transcriptional activator, and an antiviral agent. In some embodiments of the kit, the transcriptional activator is linked to a component of the programmable DNA binding protein system. In some embodiments of the kit, the antiviral agent is selected from ganciclovir, acyclovir, enciclovir, penciclovir, valacyclovir, famciclovir, and bromovinyldeoxyuridine. In some embodiments of the kit, the antiviral agent is ganciclovir (GCV). In some embodiments of the kit, the lytic EBV gene is selected from the group consisting of BZLF1, BRLF1, BGLF4, BXLF1, and BMRF1.

In some aspects, the present disclosure is a cell comprising a programmable DNA binding protein system that targets a transcriptional regulatory sequence of a lytic EBV gene, and a transcriptional activator that is linked to a component of the programmable DNA binding protein system and is capable of activating transcription of the lytic EBV gene.

In some aspects, the present disclosure is a gRNA linked to a Pumilio-FBF (PUF) domain binding sequence (PBS), wherein the gRNA targets a lytic EBV gene. In some embodiments of the gRNA, the PBS is bound to a PUF domain that is linked to a transcriptional activator.

In some aspects, the present disclosure is a ribonucleoprotein complex comprising a catalytically-inactive RNA-guided engineered nuclease bound to a gRNA that targets a transcriptional regulatory sequence of a lytic EBV gene, wherein the gRNA is linked to a Pumilio-FBF (PUF) domain binding sequence (PBS), and the PBS is bound to a PUF domain that is linked to a transcriptional activator.

### BRIEF DESCRIPTION OF THE DRAWINGS

FIG. 1 shows a flow diagram for activating transcription of an EBV immediate early lytic gene (BZLF1) using a Casilio (CRISPR-Cas9-Pumilio hybrid) transactivation complex (see WO2016148994A1, PCT/US2016/021491, incorporated herein by reference). Transcription of an EBV immediate early lytic gene in tumor cells triggering the activation of the EBV lytic cycle. The expression of immediate early proteins activates expression of downstream EBV early lytic genes (BGLF4, BXLF1) The EBV lytic cycle triggers the death of tumor cells infected with EBV. BGLF4 and BXLF1 phosphorylate and activate ganciclovir (GCV). Activated GCV kills tumor cells containing GCV as well as adjacent tumor cells that do not contain GCV.

FIGS. 2A-2B provide data demonstrating stable expression of HA-dCas9 and 3×FLAG-PUFa-p65HSF1 in C666-1 and SNU-719 cell lines. C666-1 is an EBV-associated nasopharyngeal carcinoma cell line and SNU-719 is an EBV-associated gastric adenocarcinoma cell line. FIG. 2A is an electrophoretic gel image showing that the anti-HA and anti-FLAG antibodies detected stable expression of HA-dCas9 and 3×FLAG-PUFa-p65HSF1 respectively in C666-1 and SNU-719 cell lines. FIG. 2B includes microscopy images showing the immunocytochemistry of HA-dCas9 and 3×FLAG-PUFa-p65HSF1 in C666-1 and SFU-719 cell lines.

FIGS. 3A-3B provide data demonstrating activation of EBV early lytic genes in C666-1 cells stably expressing HA-dCas9 and 3×FLAG-PUFa-p65HSF1 administered guide RNAs (gRNAs) targeting the EBV immediate early lytic genes. FIG. 3A includes microscopy images showing the immunocytochemistry of HA-dCas9, 3×FLAG-PUFa-p65HSF1, and the activation of transcription of BZLF1 and BGLF4 EBV early lytic genes. FIG. 3B is a graph showing the growth inhibition in the C666-1 cells treated with GCV, a gRNA that activates the transcription of BZLF1, or GCV and the gRNA that activates the transcription of BZLF1 compared to a control.

FIG. 4 is an electrophoretic gel image showing activation of the EBV early lytic genes BZLF1 (Zta) and BGLF4 in C666-1 cells stably expressing HA-dCas9 and 3×FLAG-PUFa-p65HSF1 administered guide RNAs (gRNAs) targeting the EBV early lytic gene BZLF1.

FIG. 5 includes flow cytometry graphs showing the expression of EBV early lytic genes in C666-1 cells stably expressing HA-dCas9 and 3×FLAG-PUFa-p65HSF1 administered guide RNAs (gRNAs) gRNA (A3), gRNA (A4), gRNA (A5), and gRNA (A6) targeting the EBV early lytic gene BZLF1.

FIGS. 6A-6B provide data demonstrating the response of C666-1 cells stably expressing HA-dCas9 and 3×FLAG-PUFa-p65HSF1 (C666-1) that were administered guide RNA A5 (gRNA A5) or control gRNA (mock) to activate the expression of EBV early lytic genes and treated with ganciclovir (GCV). FIG. 6A is a graph showing the growth rate of C666-1 cells treated with GCV or control (HCl). FIG. 6B is a graph showing the survival of C666-1 cells treated with GCV or control at 120 hours following treatment.

FIGS. 7A-7F provide data demonstrating that four gRNAs (gRNA (A3), gRNA (A4), gRNA (A5), and gRNA (A6) complementary to BZLF1 induced expression of EBV early lytic genes in EBV-associated cancer cell lines. FIG. 7A includes electrophoretic gel images showing the expression of the EBV early lytic gene BZLF1 in C666-1 cells stably expressing HA-dCas9 and 3×FLAG-PUFa-p65HSF1. FIG. 7B includes electrophoretic gel images showing the expression of the EBV early lytic gene BZLF1 in SNU-719 cells stably expressing HA-dCas9 and 3×FLAG-PUFa-p65HSF1. FIG. 7C includes flow cytometry graphs showing the number of BZLF1 expressing cells following transfection of gRNAs by flow cytometry. FIG. 7D includes microscopy images showing immunocytochemistry of the C666-1 cells transfected with BZLF1 gRNA (A5). FIG. 7E includes microscopy images showing immunocytochemistry of the SNU-719 cells transfected with BZLF1 gRNA (A5). FIG. 7F includes graphs showing the relative change in the expression of the EBV lytic genes BZLF1, BGLF4, and BLRF2.

FIGS. 8A-8J provide data demonstrating the effect of inducible expression of dCas9-Tet-on 3×FLAG-PUFa-p65HSF1-BZLF1 gRNA (A5) in C666-1 and SNU-719 cells. FIG. 8A includes electrophoretic gel images showing the expression of the EBV early lytic genes BZLF1 (Zta), BGLF4 (PK), BMRF1 (EA-D), and the EBV late lytic gene BFRF3 (VCAp1)8 in C666-1 and SNU-719 cells treated with doxycycline (DOX). FIG. 8B includes flow cytometry graphs showing the number of BZLF1 expressing cells transfected with gRNA (A5) and treated with doxycycline by flow cytometry. FIG. 8C includes microscopy images showing immunocytochemistry of C666-1 cells transfected with gRNA (A5) and treated with doxycycline that express BZLF1 (Zta). FIG. 8D includes microscopy images showing immunocytochemistry of SNU-719 cells transfected with gRNA (A5) and treated with doxycycline that express BZLF1 (Zta). FIG. 8E includes graphs showing the relative change in the expression of the EBV lytic genes BZLF1, BRLF1, BGLF4, and BLRF2 in C666-1 and SNU-719 cells with inducible expression of dCas9-Tet-on 3×FLAG-PUFa-p65HSF1-BZLF1 gRNA (A5). FIG. 8F includes microscopy images showing the expression of BZLF1, BRLF1, and BGLF4 in C666-1 cells by RNA in situ hybridization. One brown dot indicates one transcript, and clusters of signals suggest that cells are undergoing EBV lytic cycle activation. FIG. 8G includes microscopy images showing the expression of BZLF1, BRLF1, and BGLF4 in SNU-719 cells by RNA in situ hybridization. One brown dot indicates one transcript, and clusters of signals suggest that cells are undergoing EBV lytic cycle activation. FIG. 8H is a graph showing the additional toxic effect of ganciclovir (GCV) in cells inducibly expressing dCas9-Tet-on 3×FLAG-PUFa-p65HSF1-BZLF1 gRNA (A5) in C666-1 cells. FIG. 8I is a graph showing the additional toxic effect of GCV in cells inducibly expressing dCas9-Tet-on 3×FLAG-PUFa-p65HSF1-BZLF1 gRNA (A5) in SNU-719 cells. FIG. 8J is a graph showing EBER1 (?2) expression in EBV-negative AKATA cells infected with supernatant from DOX treated dCas9-Tet-on 3×FLAG-PUFa-p65HSF1-BZLF1 gRNA (A5) SNU-719 cells.

FIGS. 9A-9E provide data demonstrating the effect of reactivation of lytic cycle on DOX induced C666-1/dCas9-Tet-on-PUFa-p65HSF1-BZLF1 gRNA (3) in a mouse model. FIG. 9A is an experimental outline of producing an EBV reactivation model. Nude mice (n=8 per group) injected with 1×10⁶ C666-1 dCas9-Tet-on 3×FLAG-PUFa-p65HSF1-BZLF1 gRNA (A5) cells were treated with DOX diet (625 mg/kg) only or DOX diet (625 mg/kg) with I.P. injection of GCV (30 mg/kg) or no treatment, respectively for total 21 days. FIG. 9B is a graph showing the tumor volumes represented as average per group (n=8) of tumors. FIG. 9C includes photographs showing the growth of tumors in C666-1 dCas9-Tet-on 3×FLAG-PUFa-p65HSF1-BZLF1 gRNA (A5) mice at 25 days after subcutaneous implantation. FIG. 9D is a graph showing the tumor weight of sacrificed mice from each group. FIG. 9E includes microscopy images showing H&E staining of paraffin fixed tumor tissues from mice in each group. Scale bar is 100 μm.

FIG. 10 provides data demonstrating the protein expression of Rta (BRLF1) after transfecting BRLF1 gRNAs into cells. Six BRLF1 gRNAs (gRNA (1), gRNA (2), gRNA (3), gRNA (4), gRNA (5), and gRNA (6)) were transfected into C666-1 (FIG. 10) cells stably expressing dCas9-3×FLAG-PUFa-p65HSF1 individually. BRLF1 gRNA (2) and gRNA (3) induced BRLF1 (Rta) protein expression to a detectable level. The expression of BRLF1 protein induced BZLF1 (Zta) protein expression, which further induced the BGLF4 (PK) expression. Comparable PK expression was observed for cells treated with BRLF1 gRNA (3) to that of BZLF1 gRNA (3).

FIG. 11 provides data demonstrating that co-expression of BZLF1 and BRLF1 gRNAs induces EBV lytic reactivation synergistically in EBV-associated cancer cell lines. FIG. 11 includes electrophoretic gel images showing C666-1 HA-dCas9-3×FLAG-PUFa-p65HSF1 cells transfected with either BZLF1 gRNA (A5), BRLF1 gRNA (3) or both BZLF1 gRNA (A5) and BRLF1 gRNA (3). Cell lysate was extracted 48 hours post-transfection. Substantial expression of early proteins including PK (BGLF4) and EA-D (BMRF1) was observed when compared to that induced by individual gRNAs.

FIGS. 12A-12B provide data demonstrating induction of BGLF4 upon transfecting BGLF4 gRNAs (gRNA (1), gRNA (2), gRNA (3), gRNA (4), gRNA (5), gRNA (6), and gRNA (7)) into the EBV associated cancer cell lines. FIG. 12A includes electrophoretic gel images showing individual BGLF4 gRNAs transfected with 3×FLAG-PUFa-p65HSF1 into C666-1 expressing HA-dCas9 cells and total protein cell lysate was extracted 48 hours post-transfection. BGLF4 gRNA (2) and gRNA (5) induce PK (BGLF4) protein expression. FIG. 12B shows transfection of 3×FLAG-PUFa-p65HSF1 and a combination of BGLF4 gRNA (2) and gRNA (5) into C666-1 HA-dCas9 cells induces higher expression of EBV-PK (PK). EBV-PK expressed in cells without triggering EBV lytic reactivation as compared to that of gemcitabine-induced EBV lytic reactivation.

FIGS. 13A-13C provide data demonstrating induction of BZLF1 by a TALE system on inducing the BZLF1 expression in EBV-associated cancer cells. FIG. 13A includes electrophoretic gel images showing induction of BZLF1 in C666-1 cells transfected with four BZLF1 TALE constructs (TALE BZLF1 (1), TALE BZLF1 (2), TALE BZLF1 (3), and TALE BZLF1v(4). The expression of Zta (BZLF1), PK (BGLF4) and EA-D (BMRF1) EBV proteins is shown. FIG. 13B shows a comparison of BZLF1 gRNA (A5), BRLF1 gRNA (3), BZLF1 gRNA (3) and BRLF1 gRNA (3) gRNAs, and TALE BZLF1 (3) TALEs in C666-1 cells expressing dCas9. The expression of Zta (BZLF1), PK (BGLF4) and EA-D (BMRF1) EBV proteins is shown. FIG. 13C includes microscopy images showing immunocytochemistry staining of Zta (BZLF1) expression in C666-1 cells transfected with BZLF1 TALE.

FIGS. 14A-14C provide data demonstrating activation of EBV early lytic genes in C666-1 and SNU719 cells stably expressing HA-dCas9 administered p65HSF1 and single RNAs (sgRNAs) targeting the EBV immediate early lytic genes. FIG. 14A includes flow cytometry graphs showing the expression of EBV early lytic genes in C666-1 and SNU719 cells stably expressing HA-dCas9 administered p65HSF1 and single RNAs (sgRNAs) sgRNA1, sgRNA2, sgRNA3, and sgRNA4 targeting the EBV early lytic gene BZLF1. FIG. 14B includes electrophoretic gel images showing activation of the EBV lytic genes BZLF1 (Zta), BRLF1 (Rta), and BGLF4 (PK) in C666-1 and SNU 719 cells stably expressing HA-dCas9 administered p65HSF1 and single RNAs (sgRNAs) targeting the EBV early lytic gene BZLF1, and the anti-HA and anti-FLAG antibodies detected expression of HA-dCas9 and p65HSF1 respectively in SNU719 and C666-1 cells. FIG. 14C includes graphs showing the relative change in the expression of the EBV lytic genes BZLF1, BRLF1 and BGLF4 in C666-1 and SNU719 cells stably expressing HA-dCas9 administered p65HSF1 and single RNAs (sgRNAs) targeting the EBV early lytic gene BZLF1.

FIGS. 15A-15E provide data demonstrating the effect of inducible expression of dCas9-Tet on-p65HSF1-BZLF1sgRNA3 in SNU719, C666-1 and C17 cells. FIG. 15A includes flow cytometry graphs showing the number of BZLF1 expressing cells treated with doxycycline (DOX) by flow cytometry. FIG. 15B includes microscopy images showing immunocytochemistry of SNU719, C666-1 and C17 cells treated with doxycycline (DOX) that express BZLF1 (Zta) and EBV lytic late protein BFRF3 (VCAp18). FIG. 15C includes electrophoretic gel images showing the expression of the EBV early lytic proteins BZLF1 (Zta), BRLF1 (Rta), BGLF4 (PK) and EBV lytic late protein BFRF3 (VCAp18), the anti-HA and anti-FLAG antibodies detected expression of HA-dCas9 and p65HSF1 respectively in SNU719, C666-1 and C17 cells treated with doxycycline (DOX). FIG. 15D includes microscopy images showing the expression of BZLF1, BRLF1, BMRF1, BGLF4, BdRF1 and BLLF1 in SNU719, C666-1 and C17 cells treated with doxycycline (DOX) by RNA in situ hybridization. One brown dot indicates one transcript, and a cluster of signals suggest that cells are undergoing EBV lytic cycle activation. FIG. 15E is a graph showing LMP1, EBNA1, EBER1, BZLF1 and BRLF1 expression in EBV-negative AKATA cells infected with supernatant from DOX treated dCas9-Tet on-p65HSF1-BZLF1sgRNA3 SNU719 and C17 cells.

FIG. 16 provides data demonstrating the relative change in the expression of the EBV lytic genes BZLF1, BRLF1, BGLF4 and BLRF2 in C666-1, C17 and SNU 719 cells with stably inducible expression of dCas9-Tet on-p65HSF1-BZLF1sgRNA3.

FIGS. 17A-17E provide data demonstrating endogenous BZLF1 activation suppressed cell proliferation and induced apoptosis in DOX induced dCas9-Tet on-p65HSF1-BZLF1sgRNA3 in SNU719, C666-1 and C17 cells. FIG. 17A includes volcano plots of −log 10 (p-adj) and log 2 (Fold-change) in DOX-induced C666-1 and SNU719 dCas9-Tet on-p65HSF1-BZLF1sgRNA3 cells, using duplicate RNA-seq datasets. Values for EBV lytic genes (red) and BZLF1 mRNA are highlighted. Gene set enrichment analysis (GSEA) demonstrates that the apoptosis pathway is suppressed in both DOX-induced C666-1 and SNU719 dCas9-Tet on-p65HSF1-BZLF1sgRNA3 cells. FIG. 17B includes flow cytometry analysis showing sub G1 phase accumulation and S phase decrease in DOX induced dCas9-Tet on-p65HSF1-BZLF1sgRNA3 in C666-1, SNU719 and C17 cells. FIG. 17C includes flow cytometry analysis showing the number of active caspase-3 cells in DOX induced dCas9-Tet on-p65HSF1-BZLF1sgRNA3 in SNU719, C666-1 and C17 cells. FIG. 17D includes graphs demonstrating that the growth rate of DOX induced SNU719, C666-1 and C17 cells treated with ganciclovir (GCV) or control (HCl) within 8 days following treatment. FIG. 17E includes graphs showing monolayer colony formation in SNU719, C666-1 and C17 dCas9-Tet on-p65HSF1-BZLF1sgRNA3 cell growth after DOX induction, which showed a dramatic suppression.

FIGS. 18A-18C provide data demonstrating the effect of lytic cycle reactivation on DOX induced SNU719, C666-1 and C17 dCas9-Tet on-p65HSF1-BZLF1sgRNA3 in mouse model, respectively. FIG. 18A includes graphs and photographs showing the tumor volumes represented as average per group (n=8) of tumors. FIG. 18B includes microscopy images showing hematoxylin and eosin (H&E) and immunohistochemistry staining of paraffin fixed tumor tissues from mice in each group. Scale bar is 50 μm. FIG. 18C includes graphs showing the circulating EBV DNA detected in serum from mice in each group.

FIGS. 19A-19D provide data demonstrating no significant effect on DOX induced HeLa dCas9-Tet on-p65HSF1-BZLF1sgRNA3 cells. FIG. 19A includes flow cytometry analysis showing no significance in the number of active caspase-3 cells in DOX induced HeLa dCas9-Tet on-p65HSF1-BZLF1sgRNA3 cells. FIG. 19B includes flow cytometry analysis showing no difference in DOX induced dCas9-Tet on-p65HSF1-BZLF1sgRNA3 in HeLa cells. FIG. 19C is a graph showing the growth rate of HeLa cells treated with DOX or control (PBS) at 96 hours following treatment. FIG. 19D includes graphs showing monolayer colony formation in HeLa dCas9-Tet on-p65HSF1-BZLF1sgRNA3 cell growth after DOX induction, which showed a dramatic suppression.

FIGS. 20A-20E provide data demonstrating the effect of inducible expression of dCas9-Tet on-p65HSF1-BZLF1sgRNA3-BRLF1sgRNA3 in C666-1, SNU719 and C17 cells, showing the effect of artificial activation of both BZLF1 and BRLF1 in EBV positive nasopharyngeal carcinoma (NPC, C666-1 and C17) and gastric cancer (GC, SNU719) cells. FIG. 20A includes electrophoretic gel images showing the expression of the EBV lytic genes BZLF1 (Zta), BRLF1 (Rta), BGLF4 (PK) and BFRF3 (VCAp18), the anti-HA and anti-FLAG antibodies detected expression of HA-dCas9 and p65HSF1 respectively in SNU719, C666-1 and C17 cells treated with doxycycline (DOX). FIG. 20B includes flow cytometry graphs showing the number of BZLF1 expressing cells in DOX induced dCas9-Tet on-p65HSF1-BZLF1sgRNA3-BRLF1sgRNA3 in SNU719, C666-1 and C17 cells by flow cytometry, compared with DOX induced SNU719, C666-1 and C17 dCas9-Tet on-p65HSF1-BZLF1sgRNA3 cells. FIG. 20C includes flow cytometry analysis showing the number of active caspase-3 cells in DOX induced dCas9-Tet on-p65HSF1-BZLF1sgRNA3-BRLF1sgRNA3 in C666-1, SNU719 and C17 cells, compared with DOX induced SNU719, C666-1 and C17 dCas9-Tet on-p65HSF1-BZLF1sgRNA3 cells. FIG. 20D includes flow cytometry analysis showing the sub G1 phase accumulation in DOX induced dCas9-Tet on-p65HSF1-BZLF1sgRNA3-BRLF1sgRNA3 in SNU719, C666-1, and C17 cells. FIG. 20E is graphs showing the growth rate of dCas9-Tet on-p65HSF1-BZLF1sgRNA3-BRLF1sgRNA3 in C666-1, SNU719 and C17 cells treated with DOX or control (PBS) at 96 hours following treatment, compared with dCas9-Tet on-p65HSF1-BZLF1sgRNA3-BRLF1sgRNA3-BRLF1sgRNA3 in C666-1, SNU719 and C17 cells.

FIG. 21 provides data demonstrating BGLF4 sgRNAs induced BGLF4 gene expression. SNU-719 dCAS9-inducible p65-HSF1 were transiently transfected with 1 microgram (1 ug) of the BGLF4 sgRNA (1) or 1 ug of sgRNA (2) and in combination (0.5 ug:0.5 ug ratio (0.5×) and 1 ug:1 ug (1×)). The cells were further incubated with doxycycline to induce p65-HSF-1 expression 24 hours post-transfection. Cells were harvested at 48 hours post-doxycycline treatment and protein was extracted for Western blotting analysis. The result shows that synergistic effect of inducing BGLF4 expression could be achieved by co-expressing BGLF4 sgRNAs.

FIGS. 22A-22B provide data demonstrating the effect of TALE transactivator on activating BZLF1 expression in EBV-associated cancer cell lines C666-1 and SNU-719. Transient overexpression of BZLF1 TALEs induced EBV BZLF1 gene expression in C666-1 and SNU-719 cell lines. FIG. 22A. Different BZLF1 TALE constructs were transiently transfected into C666-1. The treatment of cells with gemcitabine and valproic acid was used as positive control. Cells were harvested and total protein was extracted for Western blotting analysis. Similar to the results using sgRNAs, the corresponding BZLF1 TALE constructs would induce BZLF1 gene expression. Quantitative RT-PCR showed that different BZLF1 TALEs induced BZLF1 gene expression in a different extent. FIG. 22B. Similar experiment was performed in SNU-719 cell lines as FIG. 22A. Both Western blotting and qPCR experiment show the induction of BZLF1 expression. TPA treatment of SNU-719 was used as positive control of lytic reactivation.

FIGS. 23A-23B provide data demonstrating the effect of TALE transactivator on activating BRLF and BGLF4 expression in EBV-associated cancer cell lines C666-1. FIG. 23A Transient overexpression of BRLF1 TALEs induced EBV BRLF1 gene expression in C666-1. Induction of BRLF1 induced BZLF1 expression indicates that the lytic reactivation of EBV was triggered. FIG. 23B. BGLF4 TALE constructs were transiently transfected into C666-1 individually or in combination. Cells were harvested and total protein was extracted for Western blotting analysis. Combination of BGLF4 TALE 1 and 2 promoted BGLF4 protein expression in C666-1 cells.

### DETAILED DESCRIPTION

The technology platform provided herein, in some aspects, relies on the unique episomal nature of the Epstein-Barr virus (EBV) genome and is used to activate a latent-to-lytic switch in EBV genes as a means for treating EBV-associated cancers, such as NPC and gastric cancer. When the latent EBV viruses are induced to enter into the lytic cycle, the immediate-early (IE) proteins—BZLF1 and BRLF1—are expressed, and these proteins activate the transcription of early and late proteins, such as BGLF4. Ectopic BZLF1 expression alone can trigger the switch from latent stage into lytic cycle in EBV-infected cells.

The antiviral drug ganciclovir (GCV) is a prodrug used in oncolytic therapy of EBV-associated cancers. Conversion of this prodrug to its cytotoxic form in cancer cells, however, requires phosphorylation by a viral kinase, such as EBV serine/threonine kinase BGLF4 and/or EBV thymidine kinase BXLF1. Thus, GCV is ineffective in cancer cells infected with EBV, if the EBV is latent. Other prodrugs for use as provided herein include, without limitation, acyclovir, enciclovir, penciclovir, valacyclovir, famciclovir, and bromovinyldeoxyuridine.

The technology provided herein, in some aspects, is used to activate selective latent EBV lytic genes in infected cancer cells exposed to GCV, thereby providing a highly efficient and effective means of killing not only the cancer cells but also bystander cells.

### Epstein-Barr Virus Infection

Methods of the present disclosure include activating transcription of an (at least one) Epstein-Barr virus (EBV) gene to induce the EBV into a lytic cycle. The methods comprise, in some embodiments, introducing into a cell infected with EBV a programmable DNA binding protein system that targets a transcriptional regulatory sequence of a lytic EBV gene, and a transcriptional activator that is linked to a component of the programmable DNA binding protein system and is capable of activating transcription of the lytic EBV gene.

Methods of the present disclosure also include activating transcription of an (at least one) EBV gene to induce the EBV into a lytic cycle by administering to a subject having a cancer associated with EBV-infection a programmable DNA binding protein system that targets a transcriptional regulatory sequence of a lytic EBV gene, and a transcriptional activator that is linked to a component of the programmable DNA binding protein system and is capable of activating transcription of the lytic EBV gene.

EBV is a herpes family virus that infects host cells. EBV infection is associated with numerous cancers, including but not limited to Burkitt's lymphoma, Kaposi's sarcoma, nasopharyngeal carcinoma, and gastric cancer. The EBV virus exists in host cells in a latent cycle of infection (latent cycle) or a lytic cycle of infection (lytic cycle). Latent EBV genes are expressed during the switch from a latent cycle to a lytic cycle. During the latent cycle, EBV virion particles are not produced and the EBV genome resides in condensed structures known as episomes in the cell. The viruses' episomal genome is replicated during a latent cycle using a host cell polymerase. The lytic cycle, or productive infection, results in the production of infectious EBV virions when the EBV genome is replicated using a viral polymerase. EBV virions are released from the host cell, killing the host cell and infecting neighboring cells to spread EBV infection.

The switch between an EBV lytic cycle and an EBV lytic cycle requires the expression of lytic EBV genes and can occur multiple (e.g., at least 1, 2, 3, 4, 5 or more) times throughout EBV infection. The EBV gene products that contribute to lytic infection to lytic infection are classified into three groups: immediate-early transactivator genes, early genes, and late genes.

Immediate-Early Transactivator Genes

In some embodiments, an EBV gene that is expressed is an immediate-early transactivator gene. A transactivator is a protein that mediates the switch from a lytic EBV cycle to a lytic EBV cycle by enhancing the expression of other immediate-early transactivator genes and downstream early genes and late genes. Immediate-early transactivator genes are the first EBV lytic genes that are transcribed in switching from lytic cycle to lytic cycle. Non-limiting examples of immediate-early transactivator genes include BZLF1 and BRLF1.

In some embodiments, an immediate-early viral transactivator gene is BZLF1 (Gene ID: 3783744). This gene is also referred to herein as Zta and EB1. The BamHI Z Epstein-Barr virus replication activator (“ZEBRA”) protein is produced following BZLF1 expression. ZEBRA binds to the lytic origin of replication of the EBV genome and interacts with the viral helicase-primase complex and the viral polymerase accessory factor BMRF1 to stimulate BRLF1, early gene, and late gene expression.

In some embodiments, an immediate-early viral transactivator gene is BRLF1 (Gene ID: 3783727). This gene is also referred to herein as Rta. The BRLF1 protein is produced following BRLF1 gene expression. BRFL1 protein stimulates the expression of BZLF1 through activating mitogen-activated protein kinases (MAPKs) and some downstream early genes and late genes by binding to a GC-rich motif present in some early gene and late gene promoters.

Early Genes

In some embodiments, a lytic EBV gene that is expressed is an early gene. Lytic EBV early genes are transcribed after immediate-early genes and before late genes. Early gene products regulate EBV viral genome replication and metabolism and block antigen processing. Non-limiting examples of early genes include: protein kinase genes, thymidine kinase genes, DNA polymerase genes, transcription factor genes, ribonucleotide reductase genes, alkaline exonuclease genes, dUTPAse genes, uracil DNA glycosylase genes, DNA polymerase accessory genes, DNA binding protein genes, primase genes, primase accessory genes, helicase genes, mRNA export factor genes, bcl-2 homolog genes, bcl-2 antagonist genes, virion genes, and immune evasion genes.

In some embodiments, a lytic EBV gene that is expressed is an early gene that encodes a protein kinase protein. Protein kinase proteins phosphorylate target proteins to stimulate or inhibit their activities. In some embodiments, a protein kinase gene is BGLF4 (Gene ID: 3783704). This gene is also referred to herein as protein kinase or PK. BGLF4 promotes nuclear lamina disassembly and its target proteins include BZLF1, BMRF1, EBNA-LP, and EBNA2.

In some embodiments, a lytic EBV gene that is expressed is an early gene that encodes a thymidine kinase gene. Thymidine kinases catalyze the transfer of a phosphate from ATP to (deoxy)thymidine monosphosphate and are required for introducing thymidine into DNA. In some embodiments, a thymidine kinase gene is BXLF1 (Gene ID: 3783741). This gene is also referred to herein as thymidine kinase or TK. BXLF1 phosphorylates thymidine and localizes to the centrosome in EBV-infected cells.

In some embodiments, a lytic EBV gene that is expressed is an early gene that encodes a polymerase accessory factor gene that is essential for EBV DNA polymerase activity. EBV DNA polymerases replicate the EBV genome. In some embodiments, an EBV DNA polymerase accessory factor gene is BMRF1 (Gene ID: 3783718). This gene is also referred to herein as early antigen diffuse component (EA-D). BMRF1 is a processivity factor that stimulates EBV replication in a complex with the EBV-DNA polymerase and the EBV deoxyribonuclease (DNase).

In some embodiments, a lytic EBV gene that is expressed is an early gene (e.g., at least one, at least two, or at least three early genes) that is selected from the group consisting of: BGLF4, BXLF1, BMRF1, BRRF1, BORF2, BaRF1, BGLF5, BLLF3, BKRF3, BALF5, BMRF1, BALF2, BSLF1, BBLF2/3, BBLF4, BMLF1, BSLF2, BHRF1, BALF1, BARF1, BFRF1, BHLF1, BHLF2, and BNLF2a.

Late Genes

In some embodiments, a lytic EBV gene that is expressed is a late gene. Late genes are the last set of EBV lytic genes that are transcribed. Late gene products regulate viral genome amplification, virion capsid assembly, release of virions from cells, and evasion of the immune system. Non-limiting examples of late genes include: tegument protein genes, major capsid protein genes, minor capsid protein genes, capsid protein genes, protease genes, 38Kd protein genes, glycoprotein genes, 53/55Kd membrane protein genes, and viral IL-10 genes.

In some embodiments, a lytic EBV gene that is expressed is a late gene that is selected from the group consisting of: BNRF1, BPLF1, BOLF1, BVRF1, BBLF1, BGLF1, BSRF1, BRRF2, BDLF2, BKRF4, BcLF1, BDLF1, BFRF3, BLRF2, BdRF1, BBRF1, BVRF2, BGLF2, BORF1, BLRF1, BLLF1, BZLF2, BKRF2, BBRF3, BXLF2, BILF1, BILF2, BALF4, BDLF3, BMRF2, BALF3, and BCRF1.

Multiple Lytic EBV Genes

In some embodiments, methods of the present disclosure include activation of multiple (e.g., 2, 3, 4, 5, 6, 7, 8 or more) lytic EBV genes simultaneously or sequentially. In some embodiments, multiple lytic EBV genes that are transcribed are from the same group of EBV lytic activation (e.g., immediate-early, early, or late). In some embodiments, multiple lytic EBV genes that are transcribed are from different groups of EBV lytic activation. In some embodiments, lytic EBV genes that are transcribed from different groups are transcribed sequentially (e.g., immediate-early, then early, then late). In some embodiments, at least one lytic EBV gene is transcribed from each group of EBV lytic activation.

Methods of the present disclosure also include synergistic EBV lytic activation by activating expression of multiple immediate-early transactivator genes. In some embodiments, immediate-early transactivator genes are BZLF1 and BRLF1. The BZLF1 and BRLF1 genes may regulate (e.g., activate or enhance) the expression of multiple downstream early genes or late genes. Multiple downstream early genes or late genes may be any genes disclosed herein. In some embodiments, multiple downstream early genes or late genes are selected from the group consisting of: genes that encode EBV protein kinase (e.g., PK, BGLF4), genes that encode EBV thymidine kinase (e.g., TK, BXLF1), and genes that encode EBV early antigen diffuse component (e.g., EA-D, BMRF1).

Synergistic EBV lytic activation is activation of multiple EBV lytic genes. Synergistic EBV lytic activation may be 2-fold to 100-fold relative to non-synergistic EBV lytic activation. In some embodiments, synergistic EBV lytic activation is 5-fold to 50-fold, 10-fold to 25-fold, 25-fold to 100-fold, or 2-fold to 25-fold relative to non-synergistic EBV lytic activation.

### Transcriptional Regulatory Sequences

Methods of the present disclosure provide activating a lytic EBV gene by introducing into a cell infected with EBV a programmable DNA binding protein system that targets a transcriptional regulatory sequence of a lytic EBV gene. A transcriptional regulatory sequence is a nucleotide sequence that regulates transcription of a gene (e.g., EBV lytic gene). In some embodiments, multiple (e.g., 2, 3, 4, 5, 6, 7, 8 or more) transcriptional regulatory sequences are targeted simultaneously. Non-limiting examples of transcriptional regulatory sequences are promoters, promoter response elements, enhancers, and silencers.

In some embodiments, a transcriptional regulatory sequence is a promoter. Promoters are DNA sequences that define where transcription of a gene (e.g., lytic EBV gene) begins. In some embodiments, a transcriptional regulatory sequence is a promoter response element (PRE). PREs are DNA sequences within promoters that are bound by transcription factors to regulate gene transcription. In some embodiments, a transcriptional regulatory sequence is an enhancer. Enhancers are short (e.g., 50-1500 base pair) DNA sequences that are bound by proteins that activate transcription (e.g., activators) to increase the transcription of a target gene (e.g., lytic EBV gene). In some embodiments, a transcriptional regulatory sequence is a silencer. Silencers are DNA sequences that are bound by proteins that repress transcription (e.g., repressors) to decrease the transcription of a target gene.

In some embodiments, a lytic BZLF1 EBV gene is activated by introducing a programmable DNA binding protein system that targets a transcriptional regulatory sequence of lytic gene BZLF1 (e.g., promoter, PRE, enhancer, or silencer) and a transcriptional activator that is linked to a component of the programmable DNA binding protein system and is capable of activating transcription of the lytic EBV gene BZLF1.

In some embodiments, a lytic BRLF1 EBV gene is activated by introducing a programmable DNA binding protein system that targets a transcriptional regulatory sequence of lytic gene BRLF1 (e.g., promoter, PRE, enhancer, and/or silencer) and a transcriptional activator that is linked to a component of the programmable DNA binding protein system and is capable of activating transcription of the lytic EBV gene BRLF1.

In some embodiments, a lytic BGLF4 EBV gene is activated by introducing a programmable DNA binding protein system that targets a transcriptional regulatory sequence of lytic gene BGLF4 (e.g., promoter, PRE, enhancer, and/or silencer) and a transcriptional activator that is linked to a component of the programmable DNA binding protein system and is capable of activating transcription of the lytic EBV gene BGLF4.

In some embodiments, a lytic BXLF1 EBV gene is activated by introducing a programmable DNA binding protein system that targets a transcriptional regulatory sequence of lytic gene BXLF1 (e.g., promoter, PRE, enhancer, and/or silencer) and a transcriptional activator that is linked to a component of the programmable DNA binding protein system and is capable of activating transcription of the lytic EBV gene BXLF1.

In some embodiments, a lytic BMRF1 EBV gene is activated by introducing a programmable DNA binding protein system that targets a transcriptional regulatory sequence of lytic gene BMRF1 (e.g., promoter, PRE, enhancer, and/or silencer) and a transcriptional activator that is linked to a component of the programmable DNA binding protein system and is capable of activating transcription of the lytic EBV gene BMRF1.

In some embodiments, multiple (e.g., 2, 3, 4, 5, 6, 7, 8 or more) transcriptional regulatory sequences are targeted sequentially. In some embodiments, where multiple transcriptional regulatory sequences are targeted, the transcriptional regulatory sequences are the same type (e.g., promoters, promoter response elements, activators, enhancers, and silencers). In some embodiments, where multiple transcriptional regulatory sequences are targeted, the transcriptional regulatory sequences are different types (e.g., promoters, promoter response elements, activators, enhancers, and silencers).

Programmable DNA Binding Proteins

The artificial transcription factor systems provided herein include programmable DNA binding proteins that can selectively bind to specific DNA target sites. Non-limiting examples of DNA-binding proteins include catalytically-inactive RNA-guided nucleases (e.g., dCas9), transcription activator-like effectors (TALEs), and zinc finger proteins (ZFPs). Commonly-known programmable DNA binding proteins often accompany a nuclease for gene editing purposes. Such programmable nucleases (also known as targeted nucleases; see, e.g., Porter et al. Compr Physiol. 2019 Mar. 14; 9(2):665-714); Kim et al. Nat Rev Genet. 2014 May; 15(5):321-34; and Gaj et al. Trends Biotechnol. 2013 July; 31(7):397-405) include, for example, zinc finger nucleases (ZFNs), transcription activator-like effector nucleases (TALENs), and RNA-guided nucleases (RGENs), such as Cas9 and Cpf1 nucleases. The artificial transcription factor systems of the present disclosure include a DNA binding protein (e.g., catalytically-inactive RGEN, TALE, or ZFP) for targeting a transcriptional activator to a target site.

In some embodiments, programmable DNA binding proteins are guided to a target sequence by protein DNA binding domains (e.g., zinc finger domains, transcription activator-like effector domains) or by guide RNAs (gRNAs).

For specific proteins described herein, the named protein includes any of the protein's naturally occurring forms, or variants or homologs that maintain the protein transcription factor activity (e.g., within at least 50%, 80%, 90%, 95%, 96%, 97%, 98%, 99% or 100% activity compared to the native protein). In some embodiments, variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring form. In other embodiments, the protein is the protein as identified by its NCBI sequence reference. In other embodiments, the protein is the protein as identified by its NCBI sequence reference or functional fragment or homolog thereof.

Catalytically-Inactive RNA-Guided Engineered Nucleases (RGENs)

CRISPR/dCas Nucleases

In some embodiments, a programmable DNA binding protein is a catalytically-inactive RNA-guided nuclease, such as a Clustered Regularly Interspace Palindromic Repeats (CRISPR/Cas) nuclease. Catalytically-inactive RGENs are modified such that they do not cleave nucleic acids. These catalytically “dead” molecules can be used, for example, to target gene regulation rather than gene disruption. RGENs can be made catalytically inactive, for example, by introducing one or more silencing mutations in the nuclease domain (see, e.g., Qi, et al., Cell 2013; 152(5): 1173-1183).

CRISPR/Cas nucleases exist in a variety of bacterial species, where they recognize and cut specific DNA sequences. The CRISPR/Cas nucleases are grouped into two classes. Class 1 systems use a complex of multiple CRISPR/Cas proteins to bind and degrade nucleic acids, whereas Class 2 systems use a large, single protein for the same purpose. A CRISPR/Cas nuclease (e.g., a catalytically-inactive CRISPR/Cas nuclease) as used herein may be selected from Cas9, Cas10, Cas3, Cas4, C2c1, C2c3, Cas13a, Cas13b, Cas13c, and Cas14 (e.g., Harrington, L. B. et al., Science, 2018).

CRISPR/Cas nucleases from different bacterial species have different properties (e.g., specificity, activity, binding affinity). In some embodiments, orthogonal RNA-guided nuclease species (e.g., catalytically-inactive RNA-guided nuclease species) are used. Orthogonal species are distinct species (e.g., two or more bacterial species). For example, a Neisseria meningitidis Cas9 and a Streptococcus thermophilus Cas9 are orthogonal relative to each other.

Non-limiting examples of catalytically-inactive bacterial CRISPR/Cas nucleases (e.g., catalytically-inactive CRISPR/Cas nucleases) for use herein include Streptococcus thermophilus Cas9, Streptococcus thermophilus Cas10, Streptococcus thermophilus Cas3, Staphylococcus aureus Cas9, Staphylococcus aureus Cas10, Staphylococcus aureus Cas3, Neisseria meningitidis Cas9, Neisseria meningitidis Cas10, Neisseria meningitidis Cas3, Streptococcus pyogenes Cas9, Streptococcus pyogenes Cas10, and Streptococcus pyogenes Cas3.

A catalytically-inactive “Cas9 nuclease” herein includes any of the recombinant or naturally-occurring forms of the CRISPR-associated protein 9 (Cas9) or variants or homologs thereof that are modified to be catalytically inactive (e.g. within at least 50%, 80%, 90%, 95%, 96%, 97%, 98%, 99% or 100% activity compared to Cas9). In some aspects, the variants or homologs have at least 90%, 95%, 96%, 97%, 98%, 99% or 100% amino acid sequence identity across the whole sequence or a portion of the sequence (e.g., a 50, 100, 150 or 200 continuous amino acid portion) compared to a naturally-occurring Cas9 nuclease. In some embodiments, a catalytically-inactive Cas9 nuclease is a modified version of the protein identified by the UniProt reference number Q99ZW2 or a variant or homolog having substantial identity thereto.

Guide RNAs (gRNAs)

RGENs are directed to a target site of interest through complementary base pairing between the target site and a guide RNA (gRNA). A guide RNA comprises (1) at least a user-defined spacer sequence (also referred to as a DNA-targeting sequence) that hybridizes to (binds to) a target nucleic acid sequence (e.g., a promoter sequence, a coding sequence, or a noncoding sequence) and (2) a scaffold sequence (e.g., a repeat sequence) that binds the programmable catalytically-inactive RGEN to guide the catalytically-inactive RGEN to the target nucleic acid sequence. As is understood by the person of ordinary skill in the art, each gRNA is designed to include a spacer sequence complementary to its target sequence. See, e.g., Jinek et al., Science, 2012; 337: 816-821 and Deltcheva et al. Nature, 2010; 471: 602-607, each of which is incorporated by reference herein. The length of the spacer sequence may vary, for example, it may have a length of 15-50, 15-40, 15-30, 20-50, 20-40, or 20-30 nucleotides. In some embodiments, the length of a spacer sequence is 15, 16, 17, 18, 19, 20, 21, 22, 23, 24, or 25+/−2 nucleotides. In some embodiments, the gRNA binds to a transcriptional regulatory sequence.

Other RGENs

Other catalytically-inactive RGENs may be used as provided herein. For example, a catalytically-inactive CRISPR-associated endonuclease from Prevotella and Francisella 1 (Cpf1) may be used. Cpf1 is a bacterial endonuclease similar to Cas9 nuclease in terms of activity. However, Cpf1 is typically used with a short (˜42 nucleotide) gRNA, while Cas9 is typically used with a longer (˜100 nucleotide) gRNA. In some embodiments, a catalytically-inactive RNA-guided nuclease is Acidaminococcus Cpf1 or Lachnospiraceae Cpf1. Any one of the foregoing RGENs may be catalytically-inactive.

RNA-Guided Tripartite Complexes

In some embodiments, a catalytically-inactive programmable nuclease is a component of an RNA-guided tripartite system that includes (1) a catalytically-inactive programmable nuclease, (2) a gRNA linked to an RNA motif that is recognized by a corresponding RNA binding protein, and (3) the corresponding RNA-binding protein. In some embodiments, the RNA-binding protein is linked to a transcriptional activator. An example of such an RNA-guided tripartite system is referred to as the ‘Casilio’ system, which herein includes a catalytically-inactive programmable nuclease (e.g., dCas9), a gRNA linked to a PUF-domain binding sequence, and a PUF domain that binds to the PUF-binding sequence (see, e.g., International Publication No. WO2016148994A and Cheng A. et al. Cell Research 2016; 26: 254-257, each of which is incorporated herein by reference). Other tripartite systems, for example, those that use other RNA motifs, may be used in accordance with the present disclosure. Non-limiting examples of other RNA motifs includes MS2, PPC, and COM motifs (see, e.g., Konermann S. et al. Nature 2015; 517: 583-588, and Zalatan J G. et al. Cell 2015; 160: 339-350, each of which is incorporated herein by reference).

In some embodiments, a gRNA is linked to one, or more than one, copy of an RNA motif (e.g., a PUF-binding sequence) that is recognized by a corresponding RNA binding protein. For example, a gRNA may be linked to 1-100, 1-50, 1-25, 5-100, 5-50, or 5-25 copies of an RNA motif. In some embodiments, a gRNA is linked to 1, 2, 3, 4, 5, 6, 7, 8, 9, 10, 11, 12, 13, 14, 15, 16, 17, 18, 19, 20, 21, 22, 23, 24, or 25 copies of an RNA motif.

The RNA motif, in some embodiments, is a PUF-binding sequence (PBS), which can be recognized and bound by a PUF domain. Non-limiting examples of PBS sequences include 5′-UGUAUGUA-3′, which can be bound by the PUF domain PUF(3-2), and 5′-UUGAUAUA-3′, which can be bound by the PUF domain PUF(6-2/7-2). Other non-limiting examples of PUF-binding sequences (and corresponding PUF domains) are provided in International Publication No. WO2016148994A.

Thus, some aspects of the present disclosure provide a tripartite complex (e.g., a ribonucleoprotein complex (catalytically-inactive nuclease bound to gRNA)) comprising an catalytically-inactive RNA-guided engineered nuclease bound to a gRNA that targets a transcriptional regulatory sequence of a lytic EBV gene, wherein the gRNA is linked to a Pumilio-FBF (PUF) domain binding sequence (PBS), and the PBS is bound to a PUF domain that is linked to a transcriptional activator.

Other aspects of the present disclosure provide a gRNA linked to a Pumilio-FBF (PUF) domain binding sequence (PBS), wherein the gRNA targets a lytic EBV gene. In some embodiments, the PBS is bound to a PUF domain that is linked to a transcriptional activator.

Zinc Finger Proteins (ZFN)

In some embodiments, a programmable DNA binding protein is a zinc finger protein (ZFP). The DNA-binding domains of ZFPs generally contain 3-6 individual zinc finger repeats that recognize 9-18 nucleotides. For example, if the zinc finger domain perfectly recognizes a 3 base pair sequence, then a 3 zinc finger array can be generated to recognize a 9 base pair target DNA sequence. Because individual zinc fingers recognize relatively short (e.g., 3 base pairs) target DNA sequences, ZFNs with 4, 5, or 6 zinc finger domains are typically used to minimize off-target DNA cutting. Non-limiting examples of zinc finger DNA-binding domains that may be used and/or modified to be catalytically inactive include Zif268, Gal4, HIV nucleocapsid protein, MYST family histone acetyltransferases, myelin transcription factor Myt1, and suppressor of tumorigenicity protein 18 (ST18). A ZFN may contain homogeneous DNA binding domains (all from the same source molecule) or a ZFN may contain heterogeneous DNA binding domains (at least one DNA binding domain is from a different source molecule).

Transcription Activator-Like Effectors (TALEs)

In some embodiments, a programmable DNA binding protein is a transcription activator-like effector (TALE). TALEs recognize and bind single target nucleotides in the DNA. TALEs found in bacteria are modular DNA binding domains that include central repeat domains made up of repetitive sequences of residues (Boch J. et al. Annual Review of Phytopathology 2010; 48: 419-36; Boch J Biotechnology 2011; 29(2): 135-136). The central repeat domains, in some embodiments, contain between 1.5 and 33.5 repeat regions, and each repeat region may be made of 34 amino acids; amino acids 12 and 13 of the repeat region, in some embodiments, determines the nucleotide specificity of the TALE and are known as the repeat variable diresidue (RVD) (Moscou M J et al. Science 2009; 326 (5959): 1501; Juillerat A et al. Scientific Reports 2015; 5: 8150). Unlike ZF DNA sensors, TALE-based sequence detectors can recognize single nucleotides. In some embodiments, combining multiple repeat regions produces sequence-specific synthetic TALEs (Cermak T et al. Nucleic Acids Research 2011; 39 (12): e82). Non-limiting examples of TALEs that may be utilized in the present disclosure include IL2RG, AvrBs, dHax3, and thXoI.

### Transcriptional Activators

In some embodiments of the present disclosure, a DNA binding protein is linked to a transcriptional activator to activate a lytic EBV gene. Transcriptional activators are polypeptides or polynucleotides that activate (or, in some embodiments, increase) the transcription of a target gene (e.g., lytic EBV gene). In some embodiments, a transcriptional activator of the programmable DNA binding protein system binds to a transcriptional regulatory sequence. Transcriptional activators, in some embodiments, bind to a target gene promoter to activate or increase transcription, although other methods of activating or increasing transcription are not excluded. Non-limiting examples of transcriptional activators include: heat shock factor 1 (HSF1) (see, e.g., Gilbert, et al. Cell 2013; 154: 442-451), p65 (see, e.g., Gilbert, et al., 2013), viral protein 16 (VP16) (see, e.g., Kaneto, et al. Diabetes 2005; 54(5): 1009-22), viral protein 64 (VP64) (see, e.g., Mali, et al., Nat. Biotechnol. 2013; 31(9): 833-838), VP64-p65-Rta (VPR) (see, e.g., Chavez, et al. Nat. Methods 2015; 12(4): 326-328), synergistic activation mediator (SAM) (see, e.g., Konerman et al. Nature 2015; 517(7536): 583-588), SunTag (see, e.g., Tanenbaum, et al. Cell 2014; 159(3): 635-646), myoblast determination protein 1 (MyoD1) (see, e.g., Weintraub, et al. Genes Dev 1991; 5(8): 1377-1386, Achaete-scute homolog 1 (Asc11), and purine rich element binding protein A (PURA).

In some embodiments, a transcriptional activator comprises or encodes a heat shock factor 1 (HSF1) transactivation domain. A transactivation domain is a protein domain that binds DNA and activates the transcription of a target gene. HSF1 protein is the primary mediator of transcriptional responses to proteotoxic stress. In some embodiments, a transcriptional activator encodes a full-length HSF1 protein. In some embodiments, a transcriptional activator encodes a fragment of a full-length HSF1 protein that retains the full (within 10%) transcriptional activation activity of the full-length HSF1 protein.

In some embodiments, a transcriptional activator comprises or encodes a p65 transactivation domain. p65 is a subunit of the NF-κB protein, which regulates DNA transcription, cytokine production, and cell survival in responses to stimuli such as stress, cytokines, free radicals, heavy metals, ultraviolet radiation, and bacterial or viral antigens. The p65 subunit (“RELA”) regulates NF-κB heterodimer formation, nuclear translocation, and activation. In some embodiments, a transcriptional activator encodes a full-length p65 protein. In some embodiments, a transcriptional activator encodes a fragment of a full-length p65 protein that retains the full transcriptional activation activity of the full-length p65 protein.

In some embodiments, a transcriptional activator comprises multiple (e.g., 2, 3, 4, 5, 6 or more) transactivation domains. In some embodiments, multiple transactivation domains are derived from the same protein (e.g., two HSF1 transactivation domains, two p65 transactivation domains). In some embodiments, multiple transactivation domains are derived from different proteins (e.g., one HSF1 transactivation domain, one p65 transactivation domains). In some embodiments, multiple transactivation domains are linked together (e.g., tandem). In some embodiments, multiple transactivation domains are linked to and separated by other components of the programmable DNA binding protein system. In some embodiments, a transcriptional activator comprises or encodes p65HSF1.

A transcriptional activator, in some embodiments, is linked to another component of the programmable DNA binding protein system. In some embodiments, a transcriptional activator is linked to another component via a linker. Linkers can be by any structure known in the art include, but not limited to: polypeptide linkers, polynucleotide linkers, covalent linkers, non-covalent linkers, and modified polynucleotide linkers. A transcriptional activator may be linked at the N-terminus and/or the C-terminus to another component of the programmable DNA binding protein system. In some embodiments, a transcriptional activator is fused to another component of the programmable DNA binding protein system (e.g., encoded as a fusion protein).

In some embodiments, a transcriptional activator is linked to a Pumilio-FBF (PUF) domain that binds to the PBS of the gRNA. A transcriptional activator may be linked at the N-terminus and/or the C-terminus to a PUF domain. In some embodiments, a transcriptional activator is linked to the catalytically-inactive RGEN. A transcriptional activator may be linked at the N-terminus and/or the C-terminus of the catalytically-inactive RGEN. In some embodiments, a transcriptional activator is linked to the gRNA. A transcriptional activator may be linked at the 5′ end and/or the 3′ end of the gRNA.

In some embodiments, expression of a component of the programmable DNA binding protein system (e.g., catalytically-inactive programmable nuclease, gRNA, transcriptional activator) is inducible. Inducible means that expression is activated by an inducing agent. Non-limiting examples of inducing agents include, doxycycline, tetracycline, isopropyl-β-D-thiogalactopyranoside (IPTG), galactose, propionate, tamoxifen, and cumate. In some embodiments, expression of a transcriptional activator is inducible.

### Antiviral Agents

Methods of the present disclosure include introducing an antiviral agent into a cell. An antiviral agent is a compound that inhibits the proliferation of or kills a virus (e.g., EBV). Non-limiting examples of antiviral agents include chemical agents, antibodies, and oligonucleotides (e.g., shRNA, siRNA, microRNA, etc.). Non-limiting examples of antiviral agents include nucleoside analogs, protein kinase inhibitors (e.g., maribavir), and thymidine derivatives (e.g., (1-[(2S,4S-2-(hydroxymethyl)-1,3-dioxolan-4-yl]5-vinylpyrimidine-2,4,(1H,3H)-dione), KAY 41, KAH-39-149).

In some embodiments, an antiviral agent is a prodrug. A prodrug is a biologically inactive compound that is metabolized in the body to produce a biologically active compound. Prodrugs may be metabolized by EBV proteins, host proteins, or EBV proteins and host proteins. In some embodiments, a prodrug is metabolized by EBV proteins. In some embodiments, a prodrug is metabolized by EBV proteins produced from activated lytic EBV genes.

In some embodiments, an antiviral agent is a nucleoside analog. A nucleoside analog is synthetic, chemically modified nucleoside that mimics endogenous nucleosides and blocks viral replication or transcription by impairing DNA/RNA synthesis or inhibiting cellular or viral enzymes involved in nucleoside metabolism. Non-limiting examples of nucleoside analogs include: ganciclovir (GCV) (see, e.g., Höcker, et al. Transpl Int 2012; 25(7): 723-731), acyclovir (ACV) (see, e.g., Pagano, et al. Am J Med 1982; 73(1A): 18-26), valgancyclovir (VGCV) (see, e.g., Höcker, et al. Transpl Int 2012; 25(7): 723-731), omaciclovir (see, e.g., Abele, et al Antimicrob. Agents Chemother 1988; 32: 1137-1142), valomaciclovir (see, e.g., Activity of Valomaciclovir in Infectious Mononucleosis Due to Primary Epstein-Barr Virus Infection (Mono6), 2007, ClinicalTrials.gov, NCT00575185, and cidofovir (see, e.g., Yoshizaki, et al. J. Med. Virol. 2008; 80: 879-882).

In some embodiments, an antiviral agent is ganciclovir (GCV). GCV is a nucleoside analog prodrug that is phosphorylated by EBV protein kinase (BGLF4) and thymidine kinase (BXLF1) to GCV-monophosphate. Host cellular kinases then catalyze the conversion of GCV-monophosphate to GCV-diphosphate and GCV-triphosphate. GCV-triphosphate is a competitive inhibitor of deoxyguanosine triphosphate (dGTP) incorporation into EBV DNA, and preferentially inhibits EBV DNA polymerase compared to cellular DNA polymerases. Other antiviral agents (e.g., prodrugs) for use as provided herein include ganciclovir, acyclovir, enciclovir, penciclovir, valacyclovir, famciclovir, and bromovinyldeoxyuridine.

The amount (e.g., dose) of antiviral agent administered to a cell with an activated lytic EBV gene is a therapeutically effective amount. Therapeutically effective amount is a dose that produces a beneficial difference (e.g., decreased number of host cells infected with EBV, decreased spread of EBV to neighboring cells, etc.) in cells compared with cells that are not administered the antiviral agent. A dose may vary based on numerous factors, including, but not limited to: administration of other antiviral agents, administration frequency, administration duration, expression level of lytic EBV genes, and other disease states or infections present in the cell.

In some embodiments, an amount of GCV (or other prodrug or other antiviral agent) administered is 1 mg/kg-100 mg/kg. In some embodiments, an amount of GCV administered is 5 mg/kg-50 mg/kg, 10 mg/kg-25 mg/kg, 2 mg/kg-50 mg/kg, or 1 mg/kg-30 mg/kg (see, e.g., Bortezomib and Ganciclovir in Treating Patients with Relapsed or Refractory Epstein Barr Virus-Positive Lymphoma, ClinicalTrials.gov, NCT00093704; Ganciclovir Plus Arginine Butyrate in Treating Patients with Cancer or Lymphoproliferative Disorders Associated with Epstein Barr Virus, ClinicalTrials.gov, NCT00006340; Study of HQK-1004 and Valganciclovir to Treat Epstein-Barr Virus—Positive Lymphoid Malignancies or Lymphoproliferative Disorders, ClinicalTrials.gov, NCT00992732). In some embodiments, an amount of GCV administered is 5 mg/kg. In some embodiments, an amount of GCV administered is 100 mg/kg (see, e.g., Westphal, et al. Cancer Research 2000; 60: 5781-5788).

### Kits

The present disclosure, in some embodiments, provides a kit. A kit may comprise, for example, a programmable DNA binding protein system that targets a transcriptional regulatory sequence of a lytic EBV gene, a transcriptional activator, and an antiviral agent. In some embodiments, a transcriptional activator is linked to a component of the programmable DNA binding protein system (such as a catalytically-inactive programmable nuclease, a protein binding domain, or a gRNA). An antiviral agent may be any antiviral agent described herein. In some embodiments, an antiviral agent is selected from ganciclovir, acyclovir, enciclovir, penciclovir, valacyclovir, famciclovir, and bromovinyldeoxyuridine. In some embodiments, an antiviral agent is ganciclovir. A lytic EBV gene may be any lytic EBV gene described herein. In some embodiments, a lytic EBV gene is selected from the group consisting of BZLF1, BRLF1, BGLF4, BXLF1, and BMRF1.

In addition to the above components, a kit may further include instructions for use of the components and/or practicing the methods. These instructions may be present in the kits in a variety of forms, one or more of which may be present in the kit. One form in which these instructions may be present is as printed information on a suitable medium or substrate, such as a piece or pieces of paper on which the information is printed, in the packaging of the kit, or in a package insert. Yet another means would be a computer readable medium, such as diskette, or CD, on which the information has been recorded. Further, another means by which the instructions may be present is a website address used via the internet to access the information at a removed site.

Components of the kits may be packaged either in aqueous media or in lyophilized form. Kits will generally be packaged to include at least one vial, test tube, flask, bottle, syringe or other container means, into which the described reagents may be placed, and suitably aliquoted. Where additional components are provided, a kit may also generally contain a second, third or other additional container into which such component may be placed.

Kits of the present disclosure may also include a means for containing the reagent containers in close confinement for commercial sale. Such containers may include injection or blow-molded plastic containers into which the desired vials are retained.

### Cells

The present disclosure, in some embodiments, provides a cell comprising a programmable DNA binding protein system that targets a transcriptional regulatory sequence of a lytic EBV gene, and a transcriptional activator that is linked to a component of the programmable DNA binding protein system and is capable of activating transcription of the lytic EBV gene.

A cell (one or more) may be any cell that is infected with EBV. EBV directly infects epithelial cells and B-cells, may be taken in by dendritic cells, and may transcytose into the lymphoid tissues of Waldeyer's ring (tonsils, adenoids, and other lymphoid tissues). In some embodiments, a cell is mammalian cell. A mammalian cell may be from any mammal including, but not limited to, a human, a mouse (see, e.g., Christian Curr Opin Virol 2017; 25: 113-118), a rat (see, e.g., Yang, et al. J. Med. Virol. 2003; 70(1): 126-130), a non-human primate (see, e.g., Wang, Curr Opin Virol, 2013; 3(3): 233-237), a dog (see, e.g., Milman, et al. Vet. Microbiol. 2011; 150(1-2): 15-20), a cat (see, e.g., Milman, et al. 2011), and a pig (see, e.g., Santoni, et al. Transplantation, 2006; 13(4): 308-317).

A cell may be any type of cell including, but not limited to: a B-lymphocyte, an epithelial cell, a natural killer cell (see, e.g., Isobe, et al. Cancer Res. 2004; 64(6): 2167-2174), a dendritic cell (see, e.g., Christian Microbiol. 2014; 5: 308), a T-lymphocyte (see, e.g., Coleman, et al. Journal of Virology 2015; 89(4); 2301-2312), a thyroid cell, a breast cell (see, e.g., Arbach, et al. Journal of Virology 2006; 80(2): 845-853), a colon cell (see, e.g., Spieker, et al. Am. J. Pathol. 2000; 157: 51-57), a renal cell (see, e.g., Becker, et al. J. Clin. Invest. 1999; 104(12): 1673-1681), a bladder cell (see, e.g., Jhang, et al. Journal of Urology 2018), a uterine cervix cell (see, e.g., Sasagawa, et al. Hum. Pathol. 2000; 31(3): 318-326), and a salivary gland cell (see, e.g., Wolf, et al. Journal of Virology 1984; 51(3): 795-798).

In some embodiments, a mammalian cell is a cancer cell. A cancer cell may be any cancer cell that is infected with EBV. Non-limiting examples of cancer cells include: nasopharyngeal carcinoma cells (e.g., C666-1), gastric cancer cells (e.g., SNU-719), Kaposi's sarcoma cells, Burkitt's lymphoma cells, Hodgkin's lymphoma cells, post-transplant lymphoproliferative disease (LPD) cells, lymphoepithelioma-like carcinoma cells, immunodeficiency-related leiomyosarcoma cells, T-cell lymphoma cells, B-cell lymphoma cells, diffuse large B-cell lymphoma cells, thyroid gland cancer cells, salivary gland cancer cells, breast cancer cells, lung cancer cells, colon cancer cells, renal cancer cells, bladder cancer cells, uterine cervix cancer cells, and squamous cell carcinoma cells.

### ADDITIONAL EMBODIMENTS

Additional embodiments of the present disclosure are encompassed by the following numbered paragraphs:

1. A method for activating a lytic Epstein-Barr virus (EBV) gene, comprising introducing into a cell infected with EBV

a programmable DNA binding protein system that targets a transcriptional regulatory sequence of a lytic EBV gene, and

a transcriptional activator that is linked to a component of the programmable DNA binding protein system and is capable of activating transcription of the lytic EBV gene.

2. A method comprising administering to a subject a programmable DNA binding protein system that targets a transcriptional regulatory sequence of a lytic EBV gene, and a transcriptional activator that is linked to a component of the programmable DNA binding protein system and is capable of activating transcription of the lytic EBV gene, wherein the subject has a cancer associated with EBV-infection.

3. The method of claim 1 or 2, wherein the programmable DNA binding protein system includes a catalytically-inactive RNA-guided engineered nuclease (RGEN) or a nucleic acid encoding a catalytically-inactive RGEN, and a gRNA that targets the transcriptional regulatory sequence or a nucleic acid encoding a gRNA that targets the transcriptional regulatory sequence.

4. The method of claim 3, wherein the gRNA binds to the transcriptional regulatory sequence.

5. The method of claim 3 or 4, wherein the gRNA is linked to a Pumilio-FBF (PUF) domain binding sequence (PBS).

6. The method of claim 5, wherein the transcriptional activator is linked to a PUF domain that binds to the PBS of the gRNA.

7. The method of claim 3 or 4, wherein the catalytically-inactive RGEN or the gRNA is linked to the transcriptional activator.

8. The method of any one of claims 3-7, wherein the catalytically-inactive RGEN is dCas9.

9. The method of claim 1 or 2, wherein the programmable DNA binding protein system includes a transcription activator-like effector (TALE) linked to the transcriptional activator.

10. The method of claim 1 or 2, wherein the programmable DNA binding protein system includes a zinc finger protein (ZFP) linked to the transcriptional activator.

11. The method of any one of the preceding claims, wherein the lytic EBV gene is an immediate-early viral transactivator.

12. The method of claim 11, wherein the immediate-early viral transactivator is selected from BZLF1 and BRLF1.

13. The method of any one of the preceding claims, wherein the lytic EBV gene is a protein kinase (PK) gene.

14. The method of claim 13, wherein the PK gene is BGLF4.

15. The method of any one of the preceding claims, wherein the lytic EBV gene is a thymidine kinase gene.

16. The method of claim 15, wherein the thymidine kinase gene is BXLF1.

17. The method of any one of the preceding claims, wherein the lytic EBV gene is essential for EBV DNA polymerase activity.

18. The method of claim 17, wherein the gene is BMRF1.

19. The method of any one of the preceding claims, further comprising introducing into the cell an antiviral agent.

20. The method of claim 19, wherein the antiviral agent is a prodrug.

21. The method of claim 20, wherein the prodrug is selected from ganciclovir, acyclovir, enciclovir, penciclovir, valacyclovir, famciclovir, and bromovinyldeoxyuridine.

22. The method of claim 21, wherein the prodrug is ganciclovir.

23. The method of any one of the preceding claims, wherein the transcriptional regulatory sequence is a promoter sequence.

24. The method of any one of the preceding claims, wherein the transcriptional activator binds to the transcriptional regulatory sequence.

25. The method of any one of the preceding claims, wherein the transcriptional activator comprises or encodes a heat shock factor 1 (HSF1) transactivation domain.

26. The method of any one of the preceding claims, wherein the transcriptional activator comprises or encodes p65HSF1.

27. The method of any one of the preceding claims, wherein expression of a component of the programmable DNA binding protein system is inducible.

28. The method of any one of the preceding claims, wherein expression of the transcriptional activator is inducible.

29. The method of any one of the preceding claims, wherein the cell is a mammalian cell.

30. The method of claim 29, wherein the cell is a cancer cell.

31. A method of synergistic Epstein Barr virus (EBV) lytic activation, comprising introducing into a cell infected with EBV (a) a programmable DNA binding protein system that targets a transcriptional regulatory sequence of EBV BZLF1 and a transcriptional regulatory sequence of EBV BRLF1, and (b) a transcriptional activator that is linked to a component of the programmable DNA binding protein system and is capable of activating transcription of the EBV BZLF1 and EBV BRLF1, wherein expression of genes regulated by EBV BZLF1 and EBV BRLF1 is at least 2-fold higher than expression of the same genes resulting from introduction of a programmable DNA binding protein system that targets only EBV BZLF1 or only EBV BRLF1.

32. The method of claim 31, wherein the genes regulated by EBV BZLF1 and EBV BRLF1 include EBV protein kinase (PK) and EBV early antigen diffuse component (EA-D).

33. A kit comprising:

programmable DNA binding protein system that targets a transcriptional regulatory sequence of a lytic EBV gene;

a transcriptional activator; and

an antiviral agent.

34. The kit of claim 33, wherein the transcriptional activator is linked to a component of the programmable DNA binding protein system.

35. The kit of claim 33 or 34, wherein the antiviral agent is ganciclovir (GCV).

36. The kit of any one of the preceding claims, wherein the lytic EBV gene is selected from the group consisting of BZLF1, BRLF1, BGLF4, BXLF1, and BMRF1.

37. A cell comprising a programmable DNA binding protein system that targets a transcriptional regulatory sequence of a lytic EBV gene, and a transcriptional activator that is linked to a component of the programmable DNA binding protein system and is capable of activating transcription of the lytic EBV gene.

38. A gRNA linked to a Pumilio-FBF (PUF) domain binding sequence (PBS), wherein the gRNA targets a lytic EBV gene.

39. The gRNA of claim 33, wherein the PBS is bound to a PUF domain that is linked to a transcriptional activator.

40. A ribonucleoprotein complex comprising a catalytically-inactive RNA-guided engineered nuclease bound to a gRNA that targets a transcriptional regulatory sequence of a lytic EBV gene, wherein the gRNA is linked to a Pumilio-FBF (PUF) domain binding sequence (PBS), and the PBS is bound to a PUF domain that is linked to a transcriptional activator.

### EXAMPLES

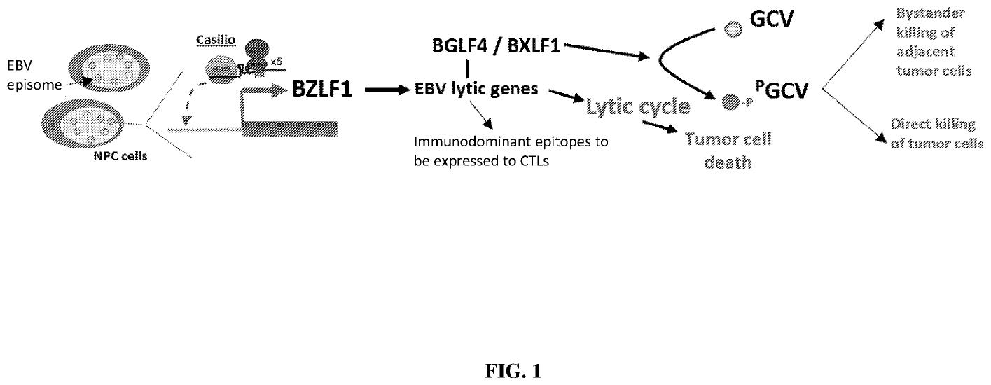

Epstein Barr virus (EBV) infects host cells and exists in either a latent phase or a lytic phase. The lytic phase occurs when the virus has commandeered host cell replication machinery to produce virion particles. The latent phase occurs when the virus is dormant and is stored in structures known as episomes in the host cell. EBV virus can be triggered to re-enter the lytic cycle in infected cells. Current cytolytic treatments that include the chemical inducers and ganciclovir (GCV) (or other prodrugs) have numerous disadvantages including low efficiency and lack of efficacy. The present disclosure provides improved technology for treating EBV-associated cancer, for example, by activating EBV lytic genes including BZLF1, BRLF1, BGLF4, and BXLF1 (FIG. 1) using programmable artificial transcription factor systems. These systems enable inducible, highly specific expression of EBV immediate early lytic genes and slow or stop cancer cell growth in vitro and in vivo.

### Example 1: Establishing Stable Cells Lines Expressing HA-dCas9-EGFP:3×FLAG-PUFa-p65HSF1 Complexes

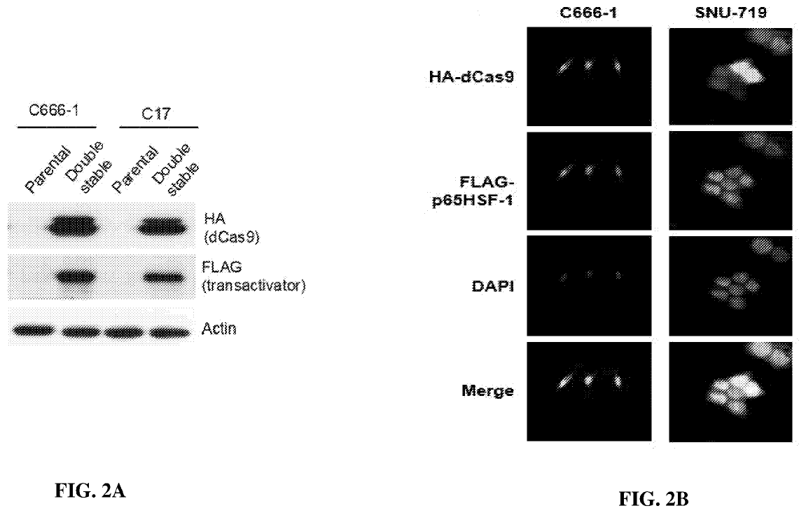

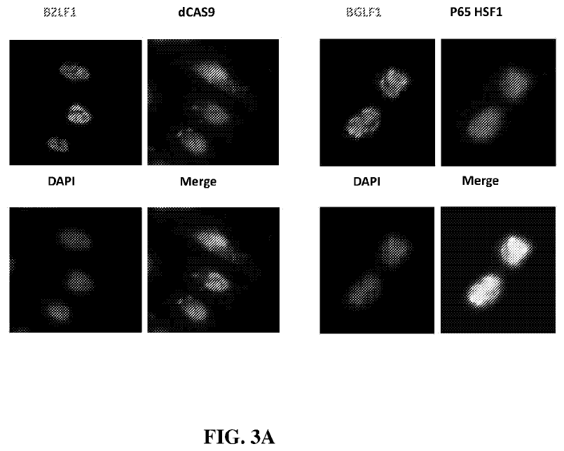

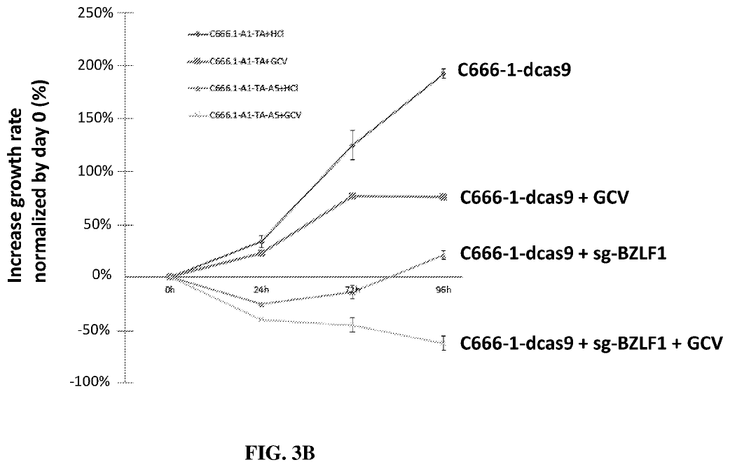

C666-1 (nasopharyngeal carcinoma cell line) and SNU-719 (gastric adenocarcinoma cell line) cells were transduced with a viral vector containing hemagglutinin tagged-nuclease deficient Cas9-EGFP complexes (HA-dCas9-EGFP) and a viral vector containing 3×FLAG tag-PUF domain a-p65HSF1 (3×FLAG-PUFa-p65HSF1) prior to selection of transfected cells. Prior to transduction, cells were seeded in 6-well plates at a density of 2×10⁶ cells/well, and 100 μL to 500 μL of viral vector was added to each well. The stable expression of each of the HA-dCas9-EGFP and 3×FLAG-PUFa-p65HSF1 constructs was confirmed by Western blot (FIG. 2A) and immunofluorescent staining with anti-BZLF1 monoclonal primary antibody and Alexa-555 secondary antibody (FIGS. 2B, 3A). Thus, C666-1 and SNU-719 cancer cell lines were established that stably expressed HA-dCas9-EGFP and FLAG-PUFa-p65HSF1.

### Materials and Methods

Establishment of a HA-dCas9-expressing cell line. The day prior to transfection, HEK293FT cells were seeded into a 10 cm petri dish at 70% density. The cells were transfected with the lentiviral packaging plasmids (pRRE (gag/pol), pRSV (rev), and VSV-G (envelope)) and a dCas9 lentiviral expression plasmid through Lipofectamine 2000 reagent (Invitrogen). Medium was exchanged at 6 hours (hrs) post-transfection. At 48 hours post-transfection, 5 mL of medium containing the lentivirus was collected and centrifuged for 5 minutes at 2,000 rpm to remove cellular debris. The supernatant was filtered utilizing a 45 μm pore filter (Millipore), and the lentivirus was collected. SNU-719 or C666-1 cells were seeded into a 10 cm petri dish at 60% density per dish, were transduced with 7 mL of the dCas9 lentivirus in culture medium supplemented with 8 μg/ml polybrene for 48 hours, and subsequently selected with Blasticidin antibiotics on the third day post-transduction.

Establishment of a HA-dCas9-EGFP and 3×FLAG-PUFa-p65HSF1-expressing cell line. The day prior to transfection, HEK293FT cells were seeded into a 10 cm petri dish at 70% density. The cells were transfected with the lentiviral packaging plasmids (pRRE (gag/pol), pRSV (rev), and VSV-G (envelope)) and a PUFa-p65HSF1 lentiviral expression plasmid through Lipofectamine 2000 reagent (Invitrogen). Medium was exchanged at 6 hours post-transfection. At 48 hours post-transfection, 5 mL of medium containing the lentivirus was collected and centrifuged for 5 minutes at 2,000 rpm to remove cellular debris. The supernatant was filtered utilizing a 45 μm pore filter (Millipore), and the lentivirus was collected. SNU-719 or C666-1 cells were seeded into a 10 cm petri dish at 60% density per dish, were transduced with 7 mL of the dCas9-PUFa-p65HSF1 lentivirus in culture medium supplemented with 8 μg/ml polybrene for 48 hours, and subsequently selected with Hygromycin antibiotics on the third day post-transduction.

Cell Culture. HEK293FT cells were cultivated in Dulbecco's modified Eagle's medium (DMEM)(Sigma) with 10% fetal bovine serum (FBS) (Gibco). C666-1 or SNU-719 cells were cultivated in RPMI-1640 (Sigma) with 10% fetal bovine serum (FBS) (Gibco), 1% Glutamax (Gibco). Incubator conditions were 37° C. and 5% CO₂.

Packaging lentivirus. HEK293FT cells were seeded into 10 cm petri dish at 70% density before being transfected with ratio of 4:1:1:1 for HA-dCas9 vector or 3×FLAG-PUFa-p65HSF1 vector:PRRE vector:VSV-G vector:RSV vector with total 10 μg plasmids. After transfection, supernatant which contained the HA-dCas9 construct or 3×FLAG-PUFa-p65HSF1 lentivirus was harvested through the 45 μm filter after 48 hours. C666-1 or SNU-719 cells were seeded into a 10 cm petri dish at 70% density before being transduced with lentivirus containing the HA-dCas9 construct or the 3×FLAG-PUFa-p65HSF1 construct. After transduction and selection for two weeks, cells were harvested for protein extraction.

Selection of transgenic cells. The selection of transgenic, e.g., multi-transgenic cells, such a single, double, triple, and/or quadruple transgenic cells, depends on the type of selectable marker used. For example, if the selectable marker protein is an antibiotic resistance protein, the selection step may include exposing the cells to a specific antibiotic and selecting only those cells that survive. If the selectable marker protein is a fluorescent protein, the selection step may include simply viewing the cells under a microscope and selecting cells that fluoresce, or the selection step may include other fluorescent selection methods, such as fluorescence-activated cell sorting (FACS) sorting.

Imaging Experiments. Cells were seeded into 6-well plates with 22 mm×22 mm×1 mm microscope cover glass at 2×10⁶ cells per well the day before imaging. The seeded cells were grown for 24 hours and then immunostained (FIG. 2B).

### Example 2: HA-dCas9-EGFP:3×FLAG-PUFa-p65HSF1 Complexes Reactivate EBV

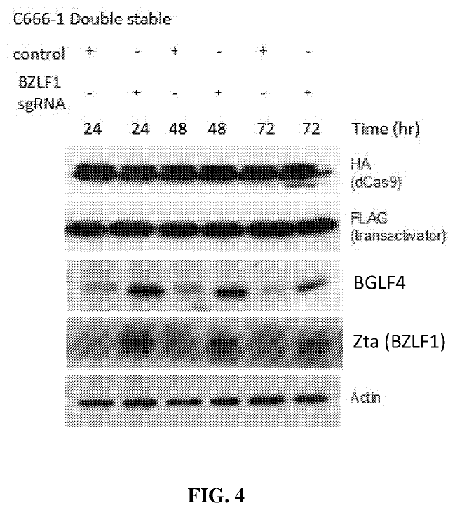

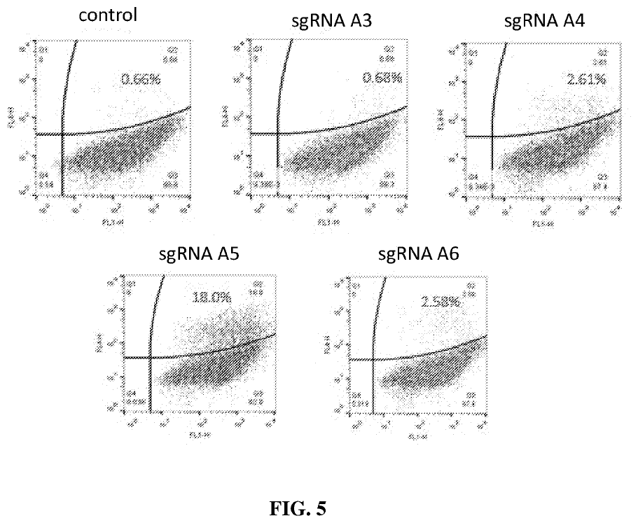

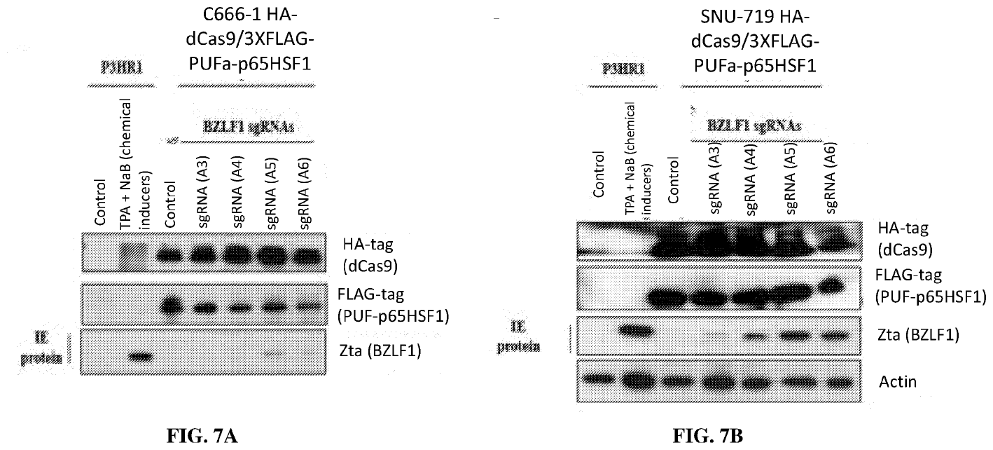

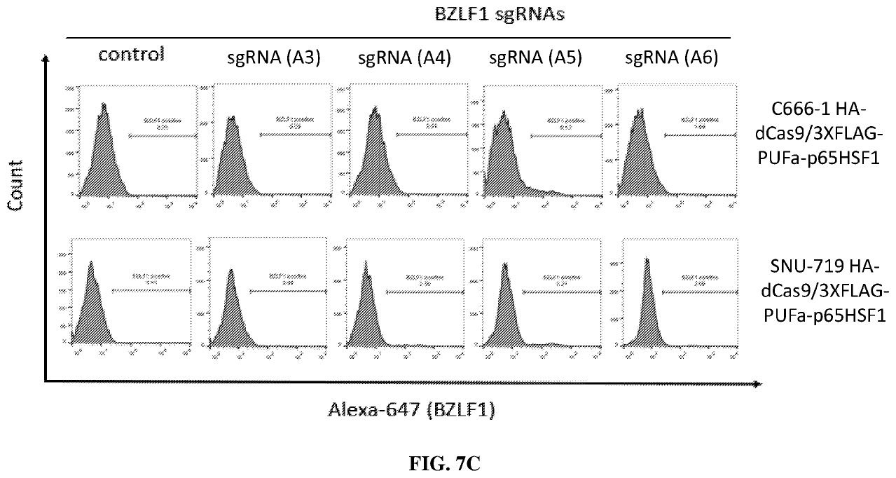

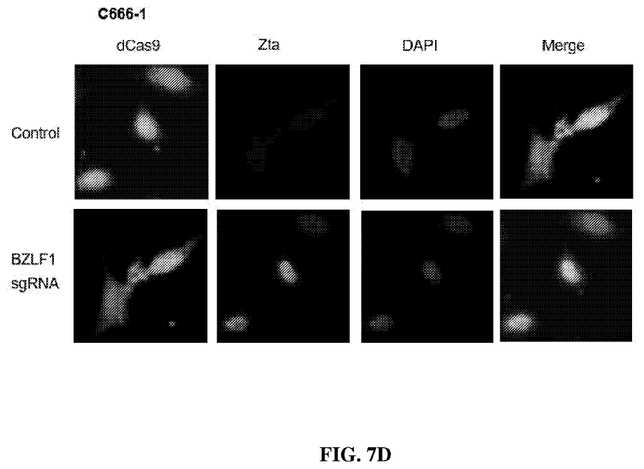

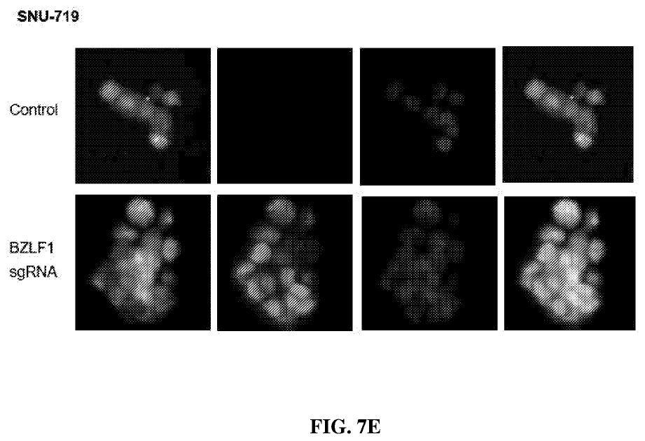

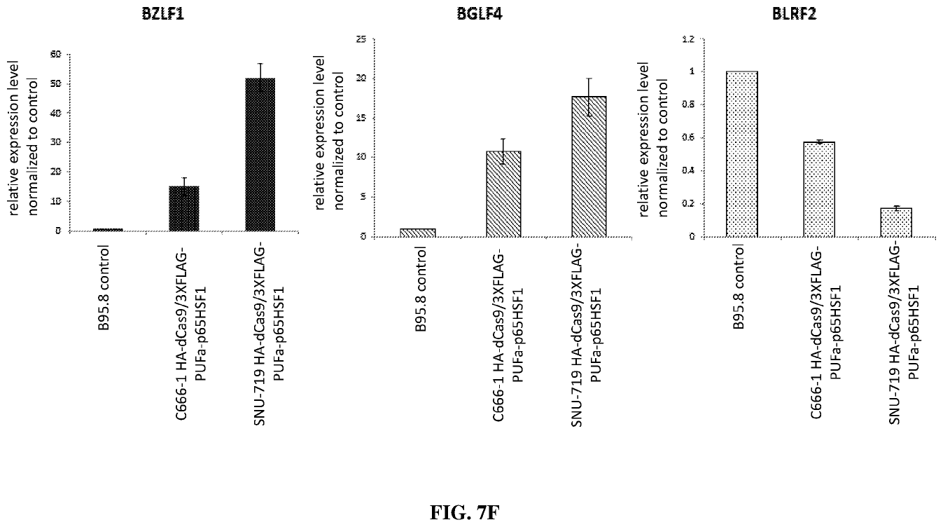

SNU-719 and C666-1 cells stably expressing HA-dCas9-EGFP and 3×FLAG-PUFa-p65HSF1 were cultivated in RPMI-1640 (Sigma) with 10% fetal bovine serum (FBS) (Gibco) and 1% GlutaMAX (Gibco) and cultured in 37° C. incubator with 5% CO2. EBV reactivation experiments were conducted with cells seeded into 6-well plates at 2×10⁶ cells per well the day before transfection. 2 micrograms (μg) of control gRNA or individual BZLF1 gRNA (A3) (SEQ ID NO: 8), gRNA (A4) (SEQ ID NO: 9), gRNA (A5) (SEQ ID NO: 10), or gRNA (A6) (SEQ ID NO: 11) gRNAs were transfected into cells with Lipofectamine 2000 (Invitrogen). After transfection, cells were grown for 48 hours and harvested for protein extraction or FACS (FIGS. 4, 5, 7A-7E). BZLF1 gRNA (A5) and BZLF1 gRNA (A6) reactivate EBV expression at detectable levels in both C666-1 and SNU-719 cells, as seen by induction of BZLF1 (Zta) protein expression. BZLF1 gRNA (A3) and BZLF1 gRNA (A4) also reactivate EBV expression at detectable levels in SNU-719 cells. Further, BZLF1 gRNA (A5) activated BZLF1 (ZTa) protein expression in both C666-1 and SNU-719 cells was shown by immunostaining (FIGS. 7D and 7E). BZLF gRNA (A5) also induces the expression of the downstream EBV genes BGLF4 and BLRF2 (FIG. 7F).

### Materials and Methods

Imaging experiments were conducted as described in Example 1.

FACS Analysis. Cells were harvested with trypsin and fixed for 15 minutes (mins) with 4% paraformaldehyde. The cells were then centrifuged at 2000×g for 5 mins and resuspended in 3% BSA for 30 mins. The resuspend cells were centrifuged again at 2000×g for 5 mins. Samples were stained with Alexa-647 conjugated anti-BZLF1 monoclonal antibody for 2 hours. Samples were analyzed on a FACSCalibur flow cytometer using CellQuest Pro software (BD Bioscience). Thousands of events were collected in each run.

Quantitative RT-PCR Analysis. Cells were harvested with trypsin, centrifuged at top speed for 1 min, and RNA was extracted using TRIzol reagent (Invitrogen). 2 μg of total RNA was used to produce a cDNA library using Superscript III Reverse Transcriptase. SYBR Green gene expression assays (Roche) were performed using the GAPDH primers as endogenous control and primers targeting the EBV lytic genes BZLF1, BGLF4, BLRF2 were used to assess EBV reactivation. Power SYBR Green master mix was used for performing real-time quantitative PCR (RT-qPCR). Each reaction was performed with 2 μL of 1:3 diluted cDNA. The RT-qPCR samples were analyzed with a Roche LC480 PCR instrument. Gene expression levels were calculated by “delta Ct” algorithm and normalized to control samples.

### Example 3: EBV Reactivation Suppresses Cancer Cells Growth In Vitro

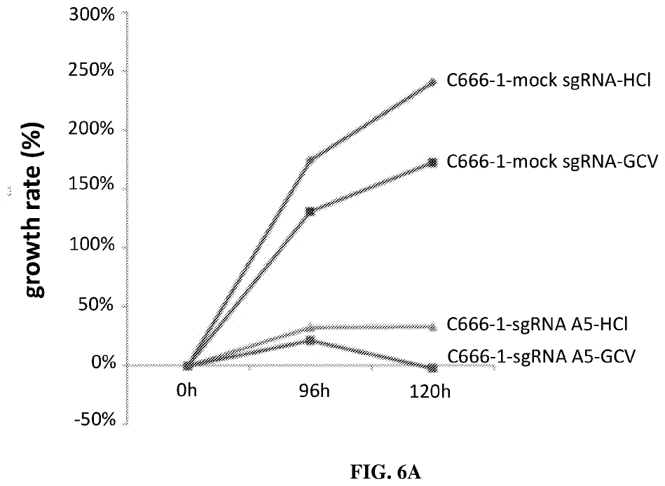

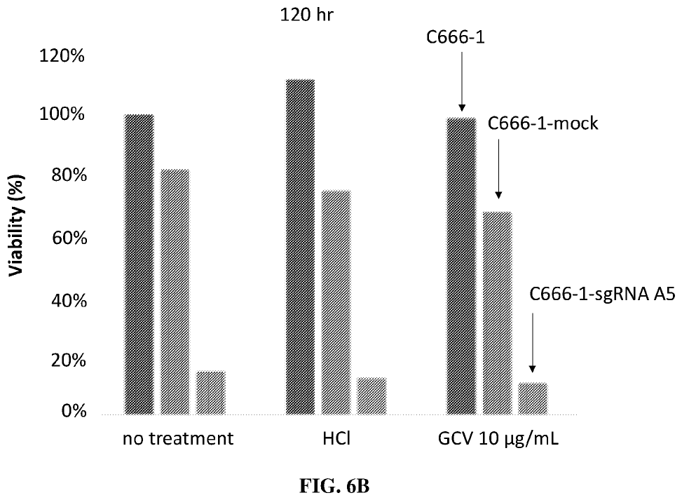

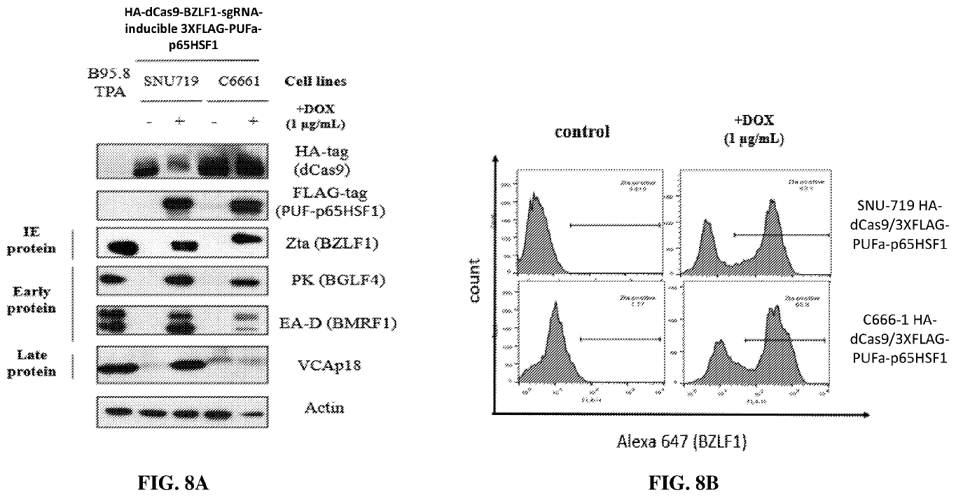

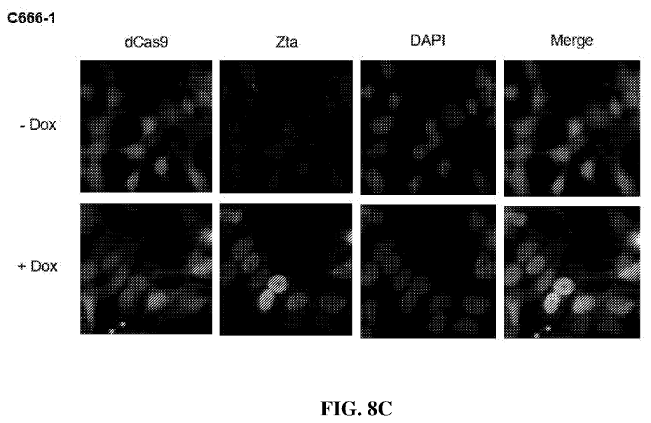

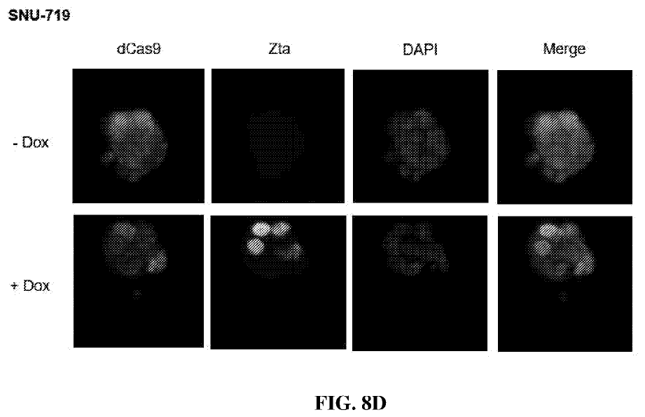

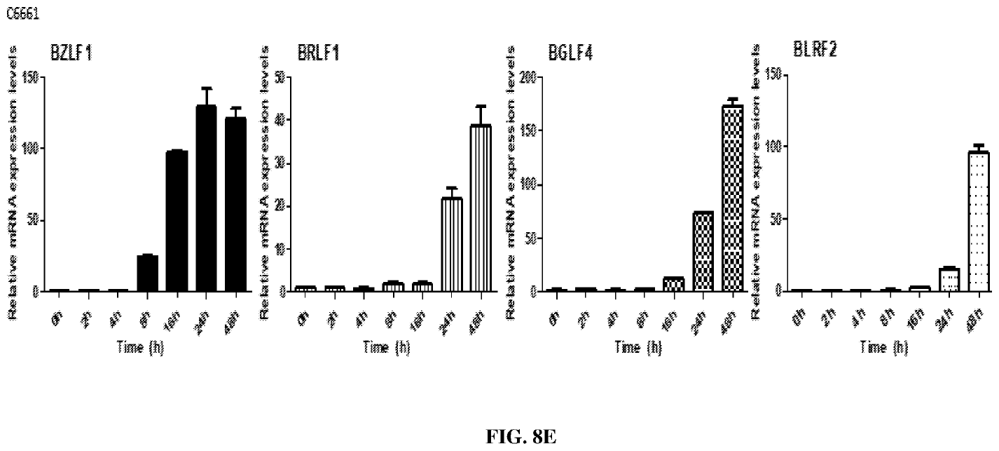

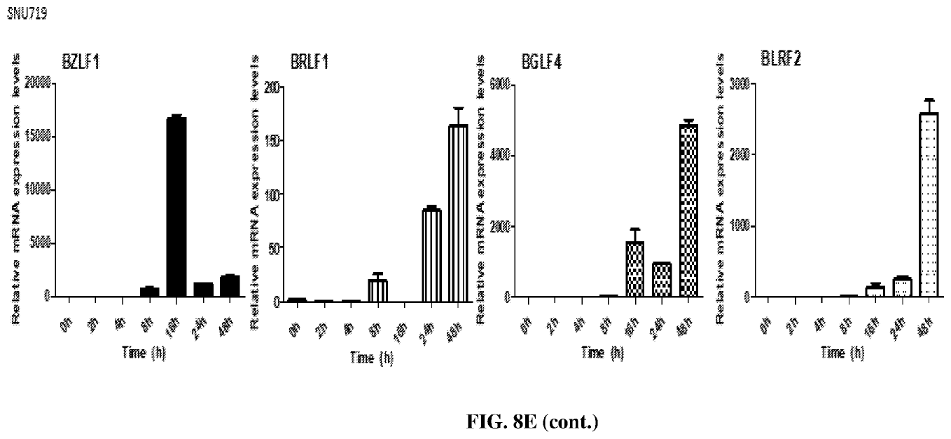

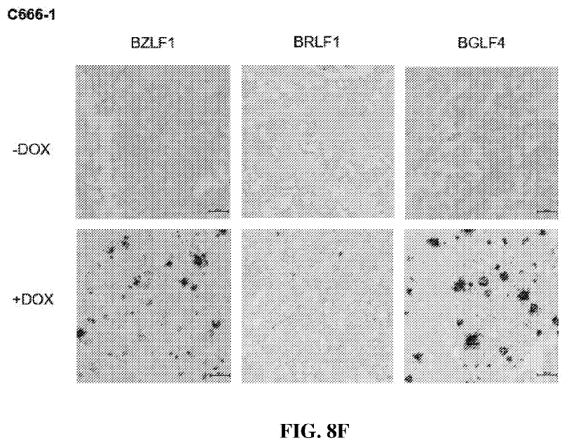

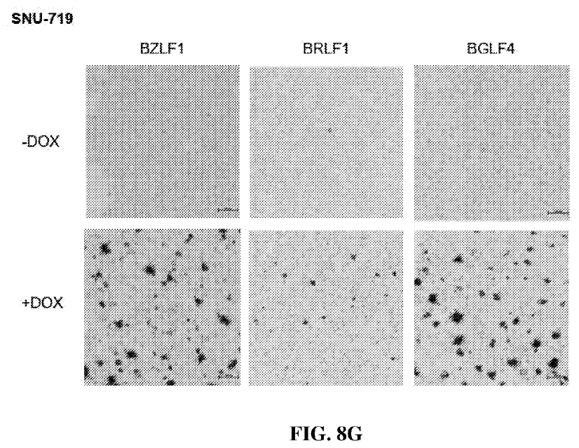

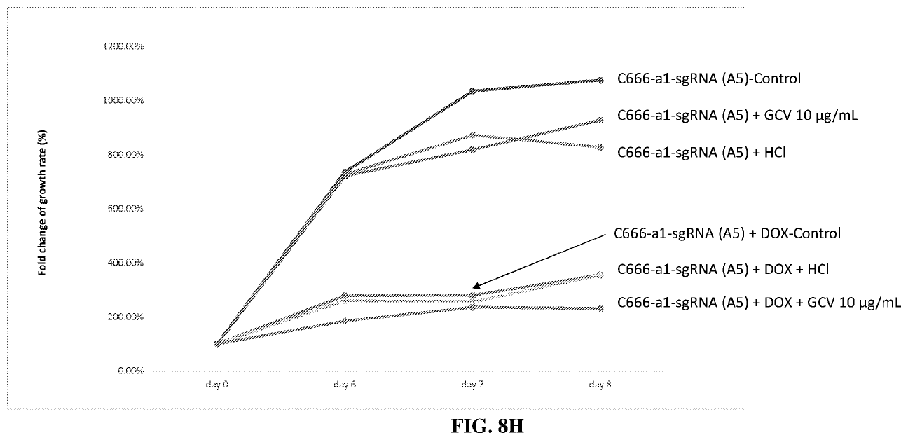

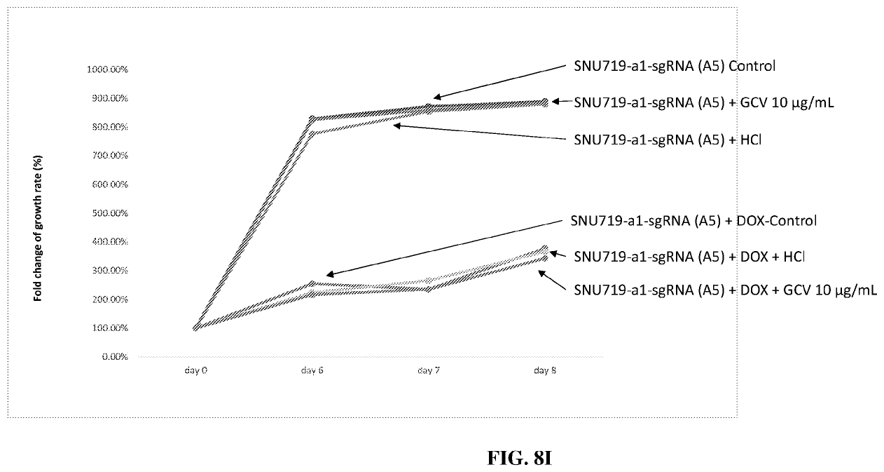

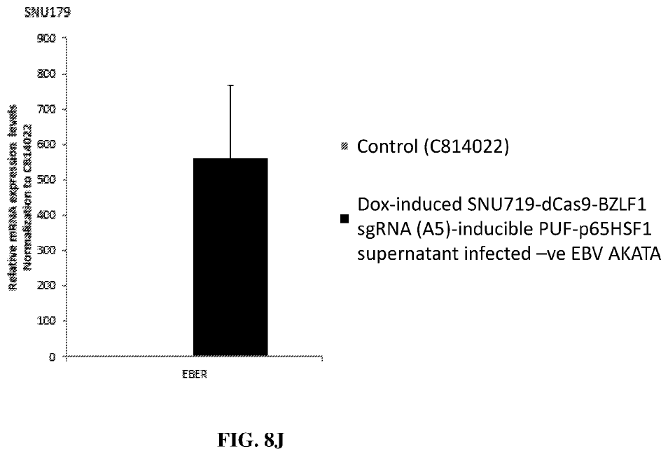

SNU-719 and C666-1 cancer cells stably expressing HA-dCas9-EGFP and BZLF1 gRNA (A5) were modified to inducibly express 3×FLAG-PUFa-p65HSF1 using a Tetracycline response element (Tet-On). Induction of 3×FLAG-PUFa-p65HSF1 expression with 1 μg/mL Doxycycline reactivated EBV, as seen through induction of the BZLF1, BGLF4, BMRF1, and BFRF3 (VCAp18) EBV lytic genes (FIGS. 8A-8G). This reactivation of EBV slows the growth rate of cells by greater than 4 fold compared with cells treated with ganciclovir (GCV) alone, a current standard treatment for EBV infection (FIGS. 3B, 6A, 8H-8I). Further, treatment with BZLF1 gRNA (A5) decreases C666-1 cell viability by 50-70% relative to control (FIG. 6B). Additionally, supernatant from SNU-719 cells expressing HA-dCas9-EGFP, gRNA BZLF1 (A5), and 3×FLAG-PUFa-p65HSF1 was able to infect and trigger the EBV gene expression (e.g. EBER1 in EBV-negative AKATA cells (FIG. 8J). Thus, the reactivated EBV lytic genes in SNU-719 and C666-1 cells slow or stop the growth and decrease cancer cell viability in vitro.

### Materials and Methods

dCas9-expressing cell lines were produced as described previously. EBV reactivation, FACS, imaging, and RT-PCR experiments were conducted as described previously.

Producing Tet-on PUFa-p65HSF1 cell line. SNU-719 or C666-1 cells stably expressing HA-dCas9-EGFP were seeded into a 60 mm culture dish at 70% density. The cells were transfected with 1 μg hyPBase (transposase) and 2 μg dox-inducible PUFa-p65HSF1 in a piggyBac vector plasmid through Lipofectamine 2000 reagent (Invitrogen). At 6 hours post-transfection, cell culture medium was exchanged. At 48 hours post-transfection, cells were selected with hygromycin antibiotics.

Producing Tet-on PUFa-p65HSF1 and BZLF1 gRNA (3) cell line. The day prior to transfection, HEK-293FT cells were seeded into a 10 cm culture dish at 70% density. The cells were transfected with the lentiviral packaging plasmids (pRRE (gag/pol), pRSV (rev), and VSV-G (envelope)) and a BZLF1 gRNA (A5) lentiviral expression plasmid using Lipofectamine 2000 reagent (Invitrogen). At 6 hours post-transfection, medium was exchanged for fresh. At 48 hours post-transfection, 5 mL of medium containing the lentivirus was collected and centrifuged for 10 minutes at 2,000 rpm to remove cellular debris. The supernatant was filtered through a 0.45-micron filter (Millipore), and the lentivirus was collected. SNU-719 dCas9-Tet-on-PUFa-p65HSF1 or C666-1 dCas9-Tet-on-PUFa-p65HSF1 cells were seeded into a 10 cm culture dish at 60% density per dish, transduced with 7 mL of the BZLF1 gRNA (A5) lentivirus in culture medium supplemented with 8 μg/mL polybrene for 48 hours, and subsequently selected with Puromycin antibiotics on the third day post-transduction.

RNA in-situ hybridization ISH (RNAscope®). Doxycycline (DOX) treated or untreated SNU-719 HA-dCas9-EGFP-Tet-on-PUFa-p65HSF1-BZLF1 gRNA (A5) cells or C666-1-HA-dCas9-Tet-on-PUFa-p65HSF1-BZLF1 gRNA (A5) cells were incubated for 48 hours. Cells were trypsinized, washed with PBS for 5 min, and centrifuged 1000 rpm for 5 min. 10% buffered formalin were used to fix cells for at least 10 min before cells were processed and embedded to generate a formalin fixed paraffin embedded cell block. Formalin-fixed paraffin-embedded (FFPE) samples were cut into 4 μm sections and baked at 60° C. for 1 hour and used within 1 week. Baked slides were de-waxed and rehydrated. 5 to 8 drops of pre-treatment reagent 1 of a pre-treatment kit were added to the de-waxed and fixed slides and incubated at room temperature for 10 min. The rack with slides was moved into boiled 1× pre-treatment reagent 2 of the pretreatment kit for 15 min, and slides were immediately transferred into a dish containing distilled water for 2 washes. 5 drops of pre-treatment reagent 3 of the pretreatment kit were added, and slides were then placed in a 40° C. pre-warmed HybEZTM oven for 30 min. About 4 drops of different specific probes (BZLF1, BRLF1, BGLF4) were placed onto each section, and slides were incubated in the 40° C. pre-warmed HybEZTM oven for 2 hours. Slides were washed with 1× wash buffer twice for 2 min at room temperature. About 4 drops of AMP1 were added to the slides, which were incubated in the 40° C. HybEZTM oven 30 min. 4 drops of AMP2 were then added, followed by a 15 min incubation in the 40° C. HybEZTM oven. 4 drops of AMP3 were then added, followed by a 30 min incubation in the 40° C. HybEZTM oven. 4 drops of AMP4 were then added, followed by a 15 min incubation in the 40° C. HybEZTM oven. 4 drops of Amp 5 was added and incubated of 30 min at room temperature. After washing, 4 drops of Amp6 were added and incubated at room temperature for 15 min. Around 120 μL of DAB was pipetted onto each section, and the slides were incubated at room temperature for 10 min and washed with distilled water. Slides were moved to 50% hematoxylin I solution for 2 min at room temperature for counterstain. Again, the slides were washed with distilled water. The slides were then dehydrated with 70% ethanol, 100% ethanol, 100% ethanol and xylene for 2 min, 2 min, 2 min and 5 min, respectively. Finally, the slides were mounted with 1 to 2 drops of cytoseal. (FIGS. 8F-8G).

Growth proliferation assay. SNU-719 HA-dCas9-Tet-on-PUFa-p65HSF1-BZLF1 gRNA (3) or C666-1 HA-dCas9-Tet-on-PUFa-p65HSF1-BZLF1 gRNA (A5) cells were seeded into individual wells of a 96-well plate for at 1×10⁵ cells per well the day before being treated with 1 μg/mL DOX and Ganciclovir (GCV). Cells were counted with the cell counting kit-8 (CCK-8, Sigma) at day 6, day 7, and day 8 (FIGS. 8H-8I).

Functional virion proliferation assay. SNU-719 HA-dCas9-Tet-on-PUFa-p65HSF1-BZLF1 gRNA (3) or C666-1 HA-dCas9-Tet-on-PUFa-p65HSF1-BZLF1 gRNA (A5) cells were seeded into 15 cm culture dish at 70% density the day before being treated with 1 μg/mL DOX. The supernatant was filtered utilizing a 45 μm pore filter (Millipore), and virus was collected by ultra-centrifuged at 20,000 rpm for 4 hours at 4° C. and resuspended in 1:30 fresh medium. EBV negative AKATA cells were treated with the medium containing virus for 96 hours, and RNA samples were extracted for quantitative RT-PCR Analysis (FIG. 8J).

### Example 4: Inducing EBV Reactivation Tumor Growth In Vivo

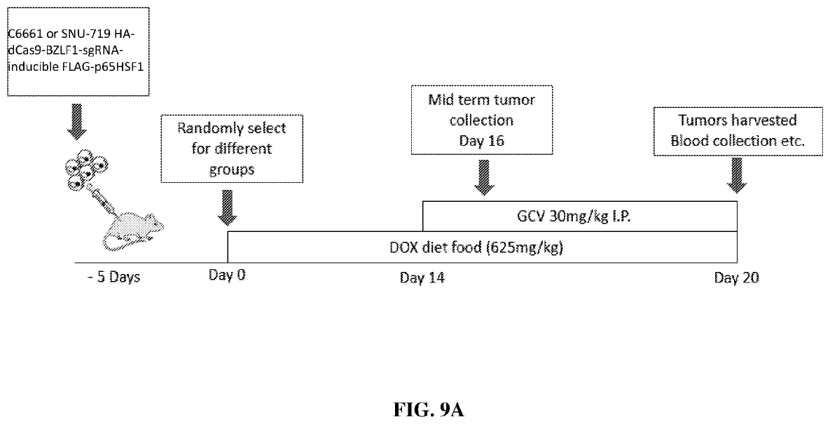

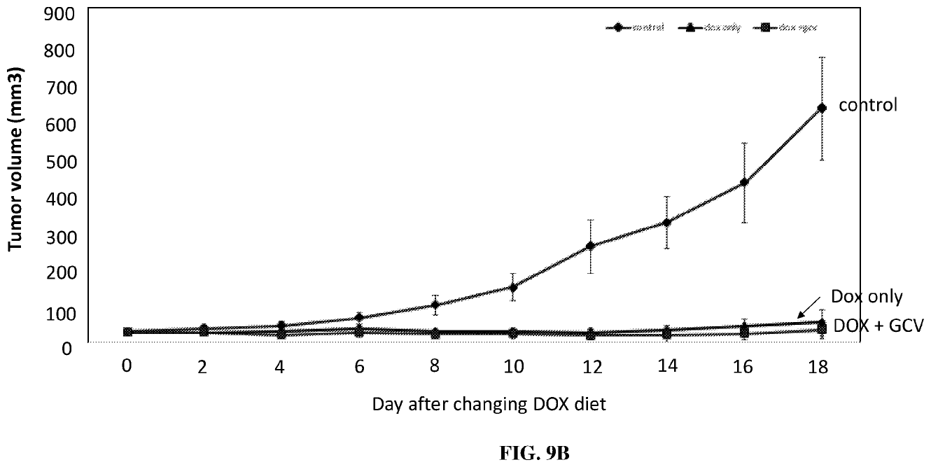

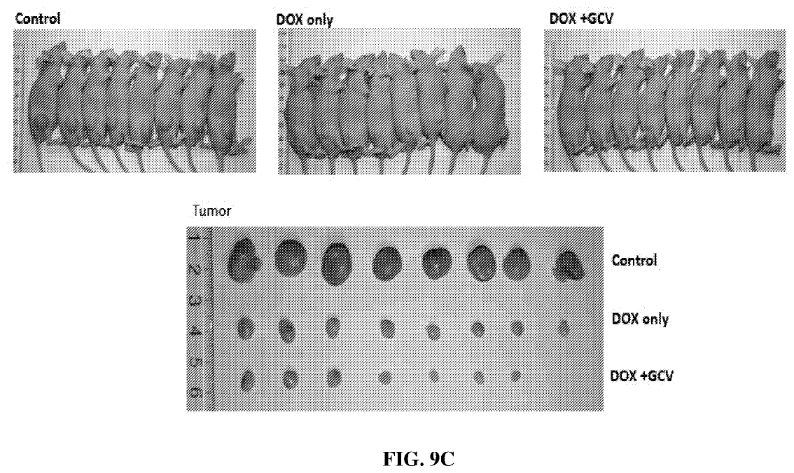

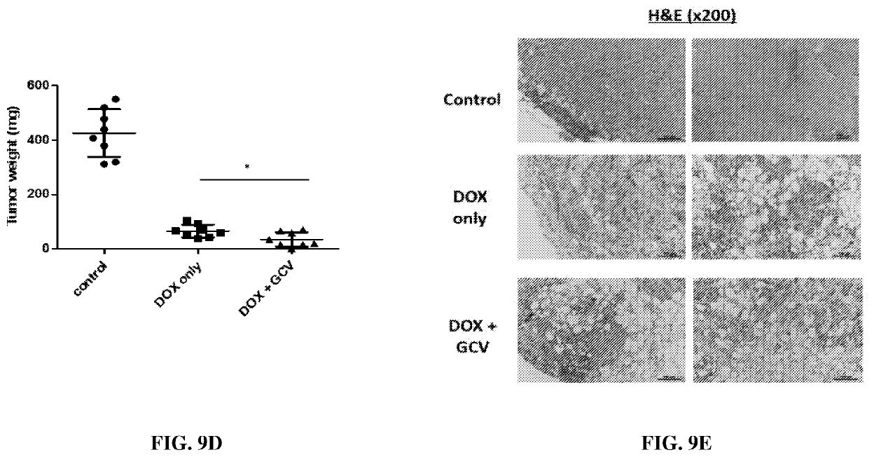

C666-1 cells stably expressing HA-dCas9-EGFP and inducibly expressing Tet-on-PUFa-p65HSF1-BZLF1 gRNA (A5) were injected into nude mice subcutaneously. The mice were treated according to the diagram in FIG. 9A. Briefly, 5 days after injection with C666-1 cells, the mice were fed a diet containing DOX to induce expression of Tet-on-PUFa-p65HSF1-BZLF1 gRNA (A5) for 20 days. 14 days after the mice began the DOX-containing diet, some mice were also treated with an intraperitoneal (I.P.) injection of GCV at 30 mg/kg. 2 days after the GCV injection, one tumor sample from each animal groups was collected for testing EBV reactivation and cell death by IHC. 4 days after the first tumor collection, all tumors were harvested and blood was collected from the mice.

Similar experiments were performed in SNU-719 cells stably expressing the HA-dCas9-EGFP and inducibly expressing Tet-on-PUFa-p65HSF1-BZLF1 gRNA (A5) with some changes in research protocols. Briefly, 12 days after cells injection the mice were fed with DOX diet to induce the expression of Tet-on-PUFa-p65HSF1-BZLF1 gRNA (A5) for 21 days. At day 9 after the mice began the DOX diet, some mice were treated with IP injection of GCV at 30 mg/kg (FIGS. 9A-9E).

Results indicate that C666-1 and SNU719 stably expressing dCas9-Tet-on-PUFa-p65HSF1-BZLF1 gRNA (A5) mice not treated with DOX or GCV develop rapidly growing tumors. Treatment with either DOX or DOX+GCV significantly slows or stops the growth of the tumors (p<0.05), indicating that reactivating the EBV lytic cycle is effective in killing tumor cells in vivo (FIGS. 9B-9E).

### Materials and Methods

Generation of mouse model. C666-1 HA-dCas9-Tet-on-PUFa-p65HSF1-BZLF1 gRNA (A5) and SNU-719 HA-dCas9-Tet-on-PUFa-p65HSF1-BZLF1 gRNA (A5) cells were mixed with an equal volume of Matrigel for of total 100 μL for subcutaneous injection to each mouse (1×10⁶ cells/mouse) at Day −5 and Day −12 respectively. (n=8 mice for each group).

Experimental design. Mice were fed with DOX diet (625 mg/kg) for two treatment groups (DOX and DOX+GCV) and the control group was fed with normal diet for 14 days. GCV was injected I.P injected to the DOX+GCV treatment group daily for 6 days (C666-1 set) and 10 days (SNU-719 set) (FIG. 9A).

Tumor size measurement. Tumor size was measured every two days during the experiment, starting from the day when the mice fed with the DOX diet. Tumor size was measured with a digital caliper along the length and width of the tumor. Length indicates the maximum horizontal dimension of the tumor and width indicates the minimum horizontal dimension of the tumor. The tumor volume was calculated followed the formula: V=Length×Width2/2, and the results are shown ±SD. FIG. 9C shows the average growth curve of tumor volume of each group. Two DOX diet fed groups showed significant growth inhibition of tumor, compared with control group.

Tumor weight measurement. Mice were sacrificed at the end of the experiment and tumors were measured with an electronic balance. The result is shown ±SD (FIG. 9D).

Hematoxylin & Eosin staining of tumor. Tumors were fixed with 10% buffered formalin to make the paraffin block. Paraffin sections of 4 μm thickness were de-waxed and rehydrated before being used for Hematoxylin & Eosin staining (FIG. 9E).

### Example 5: BRLF1 gRNAs Induce BRLF1 Expression and EBV Reactivation

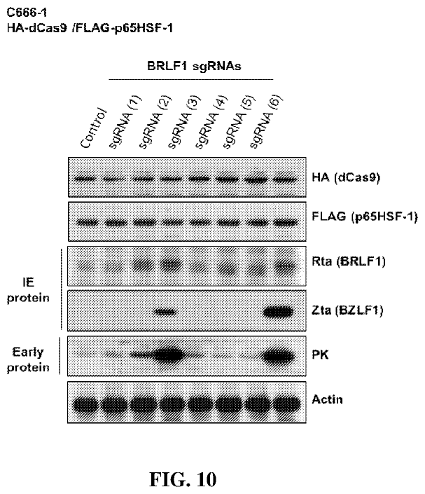

BRLF1 gRNAs trigger reactivation of EBV in vitro. Six EBV genomic loci were used to design gRNAs for BRLF1 induction and EBV reactivation. Individual gRNAs (gRNA (1), gRNA (2), gRNA (3), gRNA (4), gRNA (5), and gRNA (6) were transfected into C666-1 cells expressing HA-dCas9-EGFP and 3×FLAG-pUFa-p65HSF1. The BRLF1 gRNAs (2) and (3) induced detectable BRLF1 expression, with BRLF1 gRNA (3) inducing the strongest BRLF1 expression (FIG. 10). EBV reactivation was also triggered by the expression of BRLF1, as shown by the detection of immediate early (IE) protein BZLF1 and early protein BGLF4 (PK).

### Example 6: BRLF1 and BZLF1 gRNAs Trigger EBV Reactivation Synergistically In Vitro

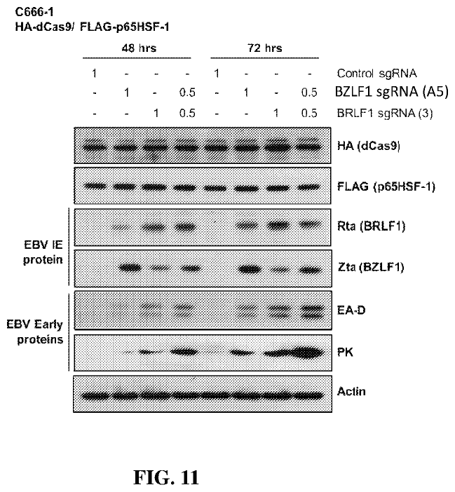

BZLF1 gRNA (A5), BRLF1 gRNA (3), or BZLF1 gRNA (3) and BRLF1 gRNA (3) in combination were transfected into C666-1 cells expressing HA-dCas9-EGFP and 3×FLAG-pUFa-p65HSF1 and the expression of EBV lytic proteins was examined Expression of BZLF1 gRNA (A5) or BRLF1 gRNA (3) induce EBV reactivation, as shown by expression of the EBV early proteins EA-D and PK (FIG. 11). Meanwhile, co-expression of both BZLF1 gRNA (A5) and BRLF1 gRNA (3) triggers a synergistic increase of EBV reactivation in the further upregulation of early proteins, EA-D and PK.

### Example 7: BGLF4 gRNAs Induce BGLF4 Expression without Triggering EBV Reactivation

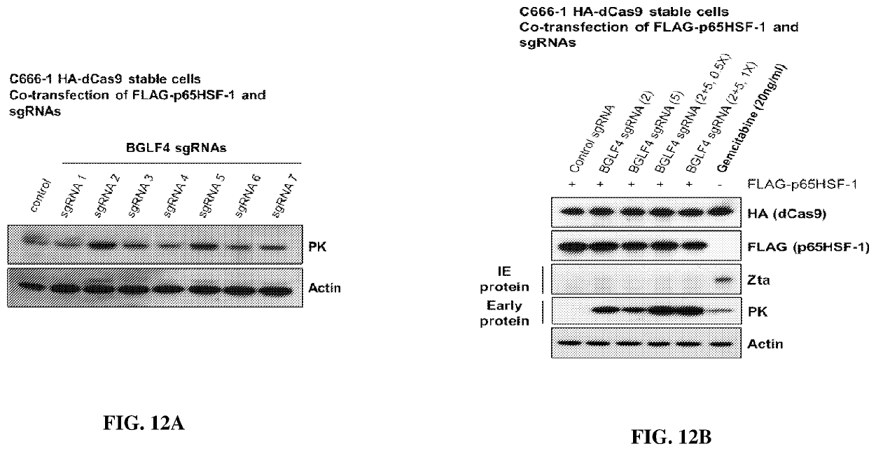

BGLF4 gRNAs were tested for triggering EBV reactivation in C666-1 cells expressing HA-dCas9-EGFP and 3×FLAG-pUFa-p65HSF1. Seven EBV genomic loci at the BGLF4 promoter were used to design the gRNAs for BGLF4 induction (gRNA (1), gRNA (2), gRNA (3), gRNA (4), gRNA (5), gRNA (6), and gRNA (7)). Individual BGLF4 gRNAs were transfected into cells and the induction of BGLF4 protein expression was studied by Western blotting. The BGLF4 gRNAs (2) and (5) activate BGLF4 expression to detectable levels (FIG. 12A). To study the combination effects of BGLF4 gRNAs, BGLF4 gRNAs were transfected into cells individually or in combination. The co-expression of BGLF4 gRNAs (gRNAs (2) and (5)) induced higher expression of BGLF4 when compared with individual gRNA-transfected cells. However, expression of BGLF4 alone did not trigger EBV reactivation as shown by the absence of BZLF1 expression (FIG. 12B). As a positive control of EBV reactivation, the C666-1 cells were treated with the chemical inducer Gemcitabine to trigger EBV reactivation. The expression of BZLF1 together with BGLF4 in cells treated with Gemcitabine implied the trigger of EBV reactivation.

### Example 8: BZLF1 TALE Triggers EBV Reactivation In Vitro Similar to Casilio System

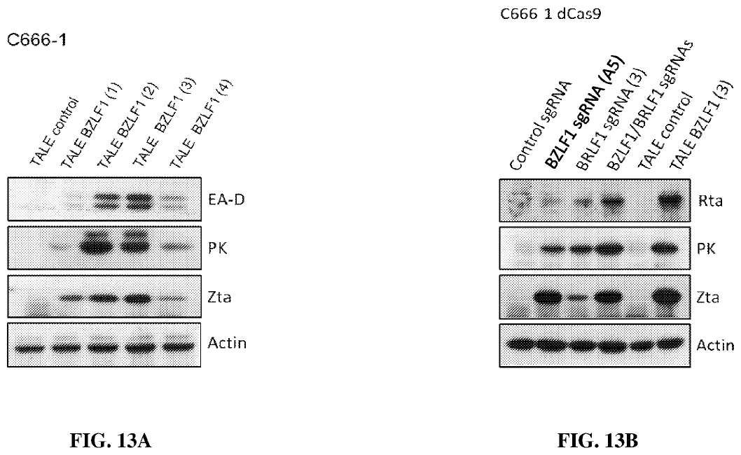

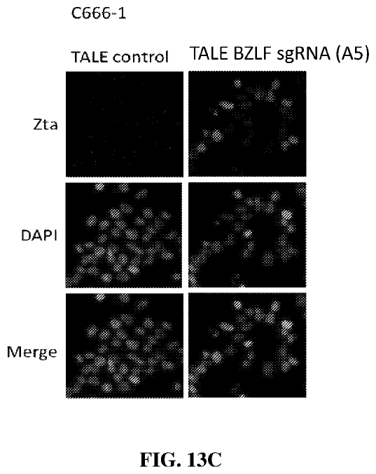

The effects of TALEs targeting BZLF1 on inducing BZLF1 expression in EBV-associated cells was studied in vitro. Four TALE constructs were designed according to the previous BZLF1 gRNA sequences and were transfected into C666-1 (nasopharyngeal carcinoma) cells to test for the induction of BZLF1 (BZLF1 TALE 1, BZLF1 TALE 2, BZLF1 TALES, and BZLF1 TALE 4). Similar to the results of BZLF1 gRNAs, the TALEs induced BZLF1 expression to different levels, and BZLF1 TALE (2) and BZLF1 TALE (3) induced the highest expression of BZLF1 protein (FIG. 13A). Concomitantly, BZLF1 TALE triggered EBV reactivation as shown by the expression of the early proteins EA-D (BMRF1) and EBV-Protein kinase (PK).

The effect of TALE BZLF1 on inducing EBV reactivation was compared with that of Casilio system. For the TALE experiment, cells were transfected with either the control TALE or the BZLF1 TALE (3). For comparison, C666-1 cells were transfected with p65HSF1 transactivator together with either BZLF1 gRNA (A5) or BRLF1 gRNA (3). Cells lysate was collected 48 hours post-transfection. TALE BZLF1 induced similar expression level of EBV lytic genes (Zta, Rta, PK) compared to BZLF1 gRNA using the Casilio system (FIG. 13B).

To study the expression of BZLF1 on individual cells, the BZLF1 TALE (3) plasmid was transfected into C666-1 cells and cells were fixed with paraformaldehyde 48 hours post-transfection. Primary antibody (anti-BZLF1) and the corresponding secondary antibody conjugated with Alexa-596 fluorochrome were used to detect the presence of BZLF1 protein in individual cells. TALE BZLF1 (3) induced expression of BZLF1 in individual cells in vitro (FIG. 13C).

### Example 9: HA-dCas9-EGFP Reactivate EBV Immediate Early Gene BZLF1 with 3×FLAG-PUFa-p65HSF1 and BZLF1 sgRNAs

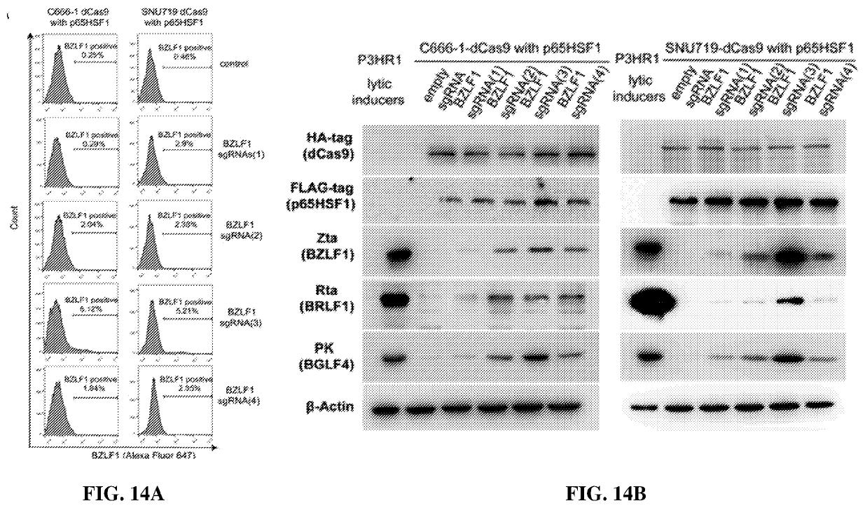

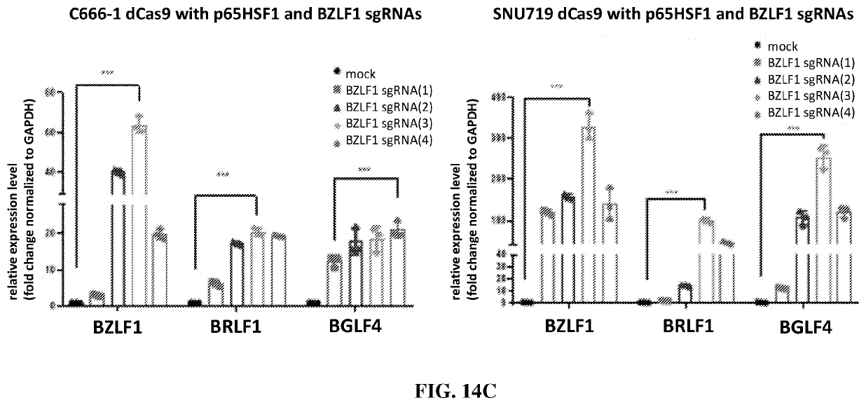

SNU-719 and C666-1 cells stably expressing HA-dCas9-EGFP were cultivated in RPMI-1640 (Sigma) with 10% fetal bovine serum (FBS) (Gibco) and 1% GlutaMAX (Gibco) and cultured in a 37° C. incubator with 5% CO2. EBV reactivation experiments were conducted with cells seeded into 6-well plates at 2×10⁶ cells per well the day before transfection. 1 microgram (μg) of p65HSF1 and 1 microgram (μg) of control sgRNA or individual BZLF1 sgRNA1 (SEQ ID NO: 8), sgRNA2 (SEQ ID NO: 9), sgRNA 3 (SEQ ID NO: 10), or sgRNA4 (SEQ ID NO: 11) were transfected into cells with Lipofectamine 2000 (Invitrogen). After transfection, cells were grown for 48 hours and harvested for FACS, protein extraction, or RNA extraction (FIGS. 14A-14C). All the BZLF1 sgRNAs reactivated EBV expression at detectable levels in both C666-1 and SNU-719 cells, as seen by induction of BZLF1(Zta) through FACS (FIG. 14A). The expression of EBV immediate early protein BRLF1 (Rta) and early protein BGLF4 (PK) were also detectable (FIG. 14B). The highest expression level of BZLF1 were shown in both protein and RNA samples which transfected with p65HSF1 and BZLF1sgRNA3 (FIGS. 14A-14C).

### Materials and Methods

dCas9-expressing cell lines were produced as described previously. EBV reactivation, FACS, electrophoretic gel images, and RT-PCR experiments were conducted as described previously

### Example 10: EBV Reactivation Suppresses Cancer Cells Growth In Vitro

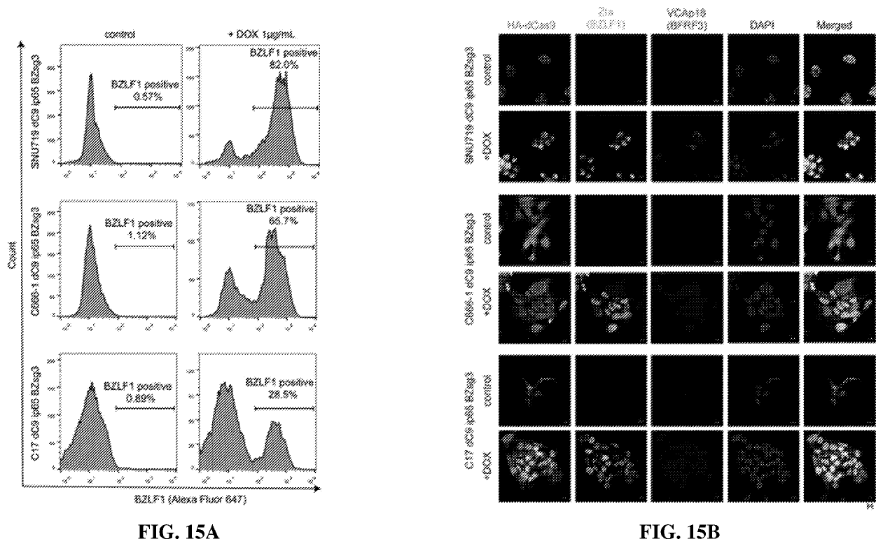

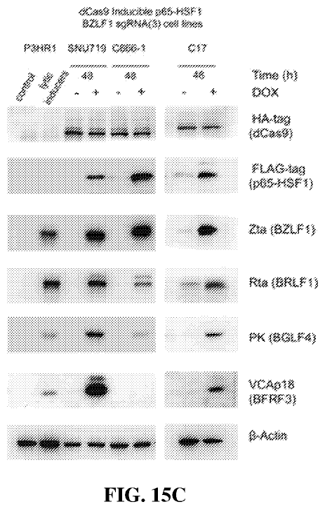

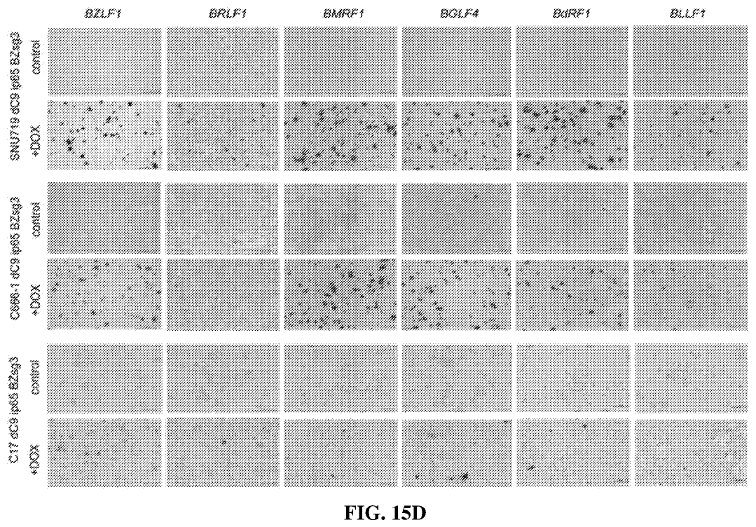

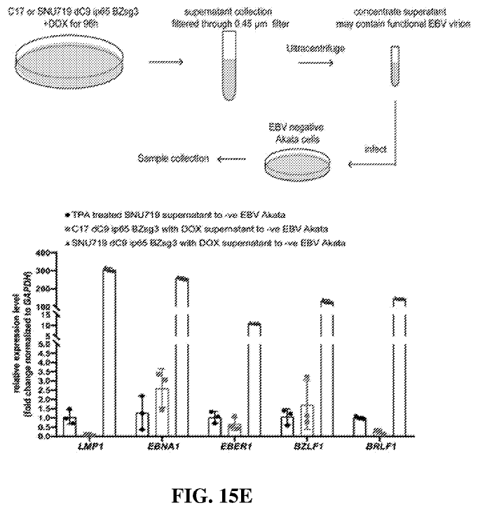

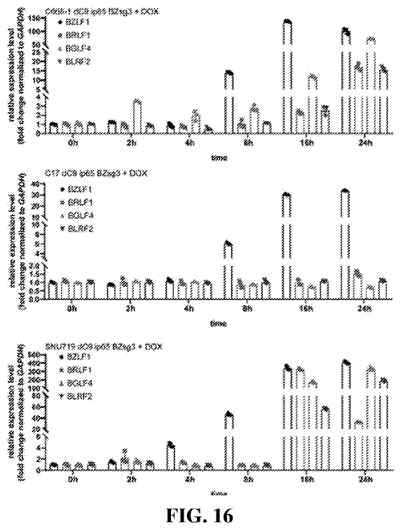

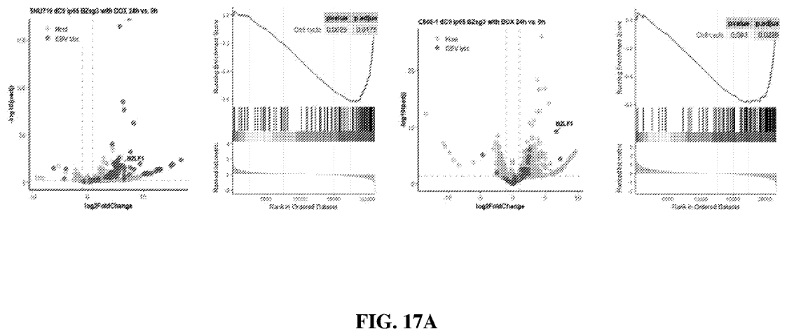

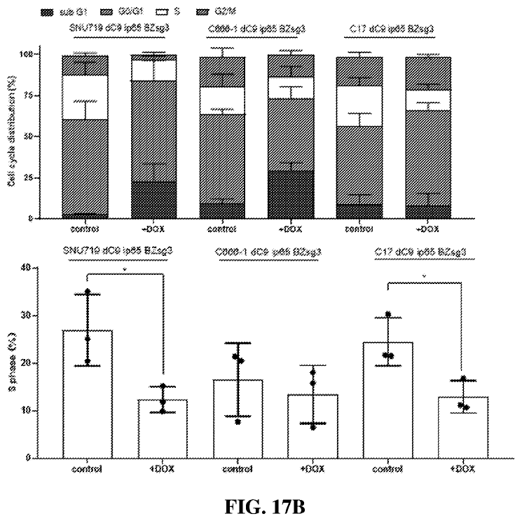

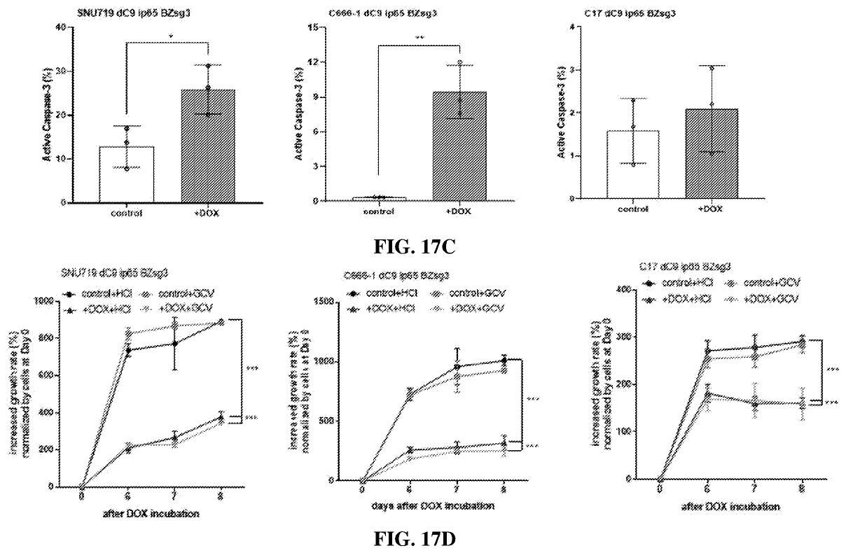

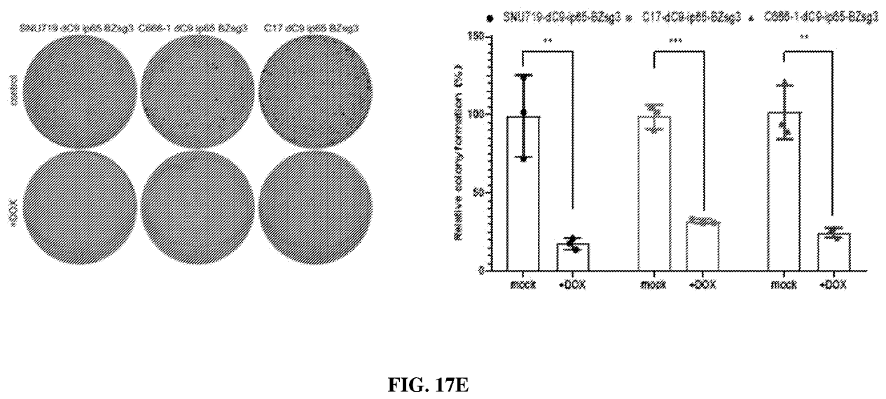

SNU-719, C666-1 and C17 (nasopharyngeal carcinoma) cancer cells stably expressing HA-dCas9-EGFP and BZLF1 sgRNA3 were modified to inducibly express 3×FLAG-PUFa-p65HSF1 using a Tetracycline response element (Tet-On). Induction of 3×FLAG-PUFa-p65HSF1 expression with 1 μg/mL Doxycycline (DOX) reactivated BZLF1 (Zta) (FIGS. 15A-15B). It also induces the expression of other EBV lytic genes including BRLF1 (Rta), BGLF4 (PK), BFRF3 (VCAp18), BMRF1, BdRF1 and BLLF1 (FIGS. 15C-15D, 16). Supernatant from DOX treated dCas9-Tet on-p65HSF1-BZLF1 sgRNA3 SNU-719 and C17 cells was able to infect the EBV-negative AKATA cells and the EBV gene expression was detectable in the infected EBV-negative AKATA cells (e.g. LMP1, EBER1, EBNA1, BZLF1, BRLF1) (FIG. 15E). Additionally, the reactivation of EBV suppressed the cell proliferation, induced apoptosis, and slowed the growth of cells in vitro (FIGS. 17A-17E).

### Materials and Methods

dCas9-Tet on-p65HSF1-BZLF1sgRNA3 cell lines were produced as described previously. EBV reactivation, FACS, electrophoretic gel images, RT-PCR experiments, RNAscope in situ hybridization, growth proliferation assay and functional virion proliferation assay were conducted as described previously

RNA-seq analysis. C666-1 and SNU-719 dCas9-Tet on-p65HSF1-BZLF1 sgRNA3 cells were seeded as 60% density in 6-well plates the day before treatment. RNA samples were collected at 0, 8, 16, 24 hrs after DOX 1 mg/mL of DOX treatment. Two micrograms (μg) RNA was used for the preparation of the ribodepleted treatment eukaryotic strand-specific RNA library according to the manufacturers' instructions. The libraries were then subjected to paired-end sequencing with the Hiseq-PE150 platform (Illumina). RNA sequencing transcript abundance was aligned to human reference genome (hg38) by HISAT2, and annotated using StringTie with the GRCh38 transcript reference genome annotation. DESeq2 was used for the differential expression analysis with treatment, time as covariates. Geneset enrichment analysis was performed, pathways with p-adj values <0.05, q-values <0.05 and absolute NES values >1 were considered significantly enriched.

Active caspase-3 analysis. To detect the apoptosis in DOX treated dCas9-Tet on-p65HSF1-BZLF1sgRNA3 SNU719 and C17 cells, active caspase-3 apoptosis kit (BD Pharmingen #550914) was used. Untreated and treated cells were trypsinized and then fixed with fixation buffer in ice for 20 mins and washed with washing buffer twice. Cells were then stained with the PE conjugated rabbit anti active caspase-3 antibody in dark for 30 mins. Stained cells were collected with BD LSRFortessa.

Cell cycle analysis. After 96 hours from induction of DOX, dCas9-Tet on-p65HSF1-BZLF1sgRNA3 SNU719 and C17 cells were trypsinized and then fixed with 75% cold ethanol for 2 hrs at −20° C. Cells were washed three times with cold PBS and then resuspended in PBS containing 100 μg/mL RNase A and 10 μg/mL propidium iodide for 10 min at room temperature. Stained cells were collected with BD LSRFortessa.

Clonogenic assays. Cells were seeded at a density of 500 cells/well in 6-well plates the day before treatment. Then cells were stained with 0.5% crystal violet 14 days after treatment, and at least 50 cells were considered as a single colony

### Example 11: Inducing EBV Reactivation Tumor Growth In Vivo

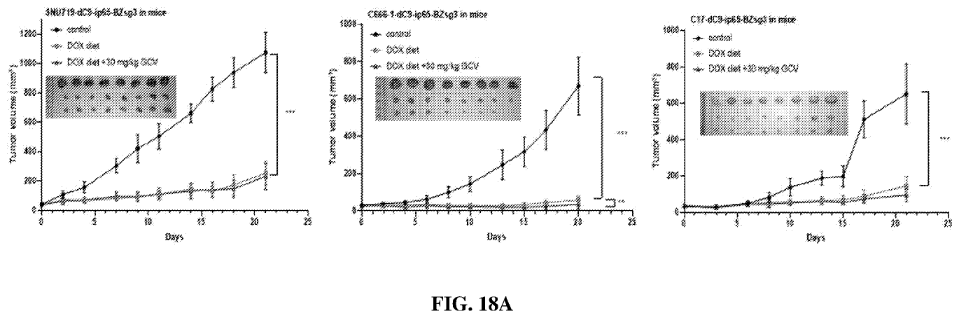

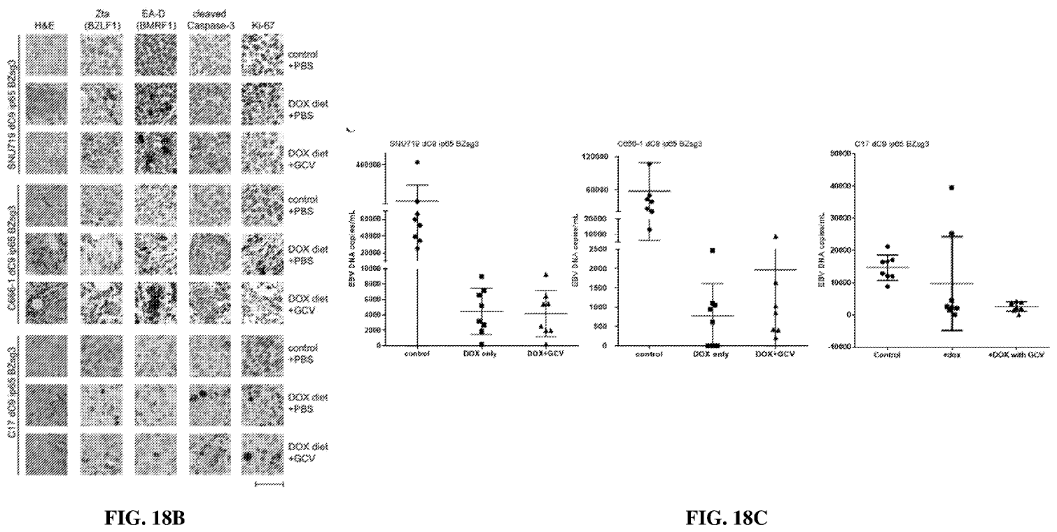

SNU-719, C666-1 and C17 cells stably expressing dCas9-Tet on-p65HSF1-BZLF1 sgRNA3 were injected into nude mice subcutaneously. 5 days after injection, the mice were fed a diet containing DOX to induce expression of Tet-on-PUFa-p65HSF1-BZLF1 sgRNA3 for 20 days. 12 to 14 days after the mice began the DOX-containing diet, some mice were also treated with an intraperitoneal (I.P.) injection of GCV at 30 mg/kg. 6 to 7 days after GCV injection, all tumors were harvested, and blood was collected from the mice. Results indicate that nude mice injected with SNU-719, C666-1 and C17 cells stably expressing dCas9-Tet-on-PUFap65HSF1-BZLF1 sgRNA3 and not treated with DOX or GCV develop rapidly growing tumors. Treatment with either DOX or DOX+GCV significantly slows or stops the growth of the tumors (p<0.05), indicating that reactivating the EBV lytic cycle is effective in killing tumor cells in vivo (FIGS. 18A-18C).

### Materials and Methods

Generation of mouse model, experimental design, tumor size measurement, and Hematotoxylin & Eosin staining of tumor were conducted as described previously.

Immunohistochemistry (IHC) Paraffin sections of 4 μm thickness were de-waxed and retrieval with citrate buffer. The slides were then blocked with 1% BSA for 30 mins at room temperature and then washing with TBS 3 mins for 3 times. Rabbit anti cleaved caspase-3 antibody (9664, Cell signaling) (1:100), mouse anti-Ki67 (550609, BD Biosciences) (1:2000), mouse anti-BZLF1 antibody (1:100) (Santa Cruz), mouse anti-BMRF1 antibody (1:200), and goat anti-BFRF3 antibody (1:4000) (Invitrogen) were stained the sections overnight at room temperature. DAB substrate was prepared to add to the slides in which the secondary antibodies were removed.

EBV DNA load in whole blood after treatment. The whole blood was collected from mice, stayed at room temperature for 30 mins and was then subjected to centrifugation at 2000 g for 15 min to collect the serum. DNA samples were extracted from serum using the DNA blood mini kit (Qiagen). A final elution volume of 20 μL was used for subsequent experiments. The primers specific targeting BamHI-W region were used: W-44F (5′-CCCAACACTCCACCACACC-3′, SEQ ID NO: 35) and W-119R (5′-TCTTAGGAGCTGTCCGAGGG-3′, SEQ ID NO: 36), and primers were synthesized by Life Technologies, Inc. A specific TaqMan fluorescent probe W-67T [59-(FAM) CACACACTACACACACCCACCCGTCTC (TAMRA)-39, SEQ ID NO: 37] was used.

### Example 12: HA-dCas9-EGFP: 3×FLAG-PUFa-p65HSF1: BZLF1 sgRNA3 Complexes do not Show Effect on EBV-Negative Cell Line

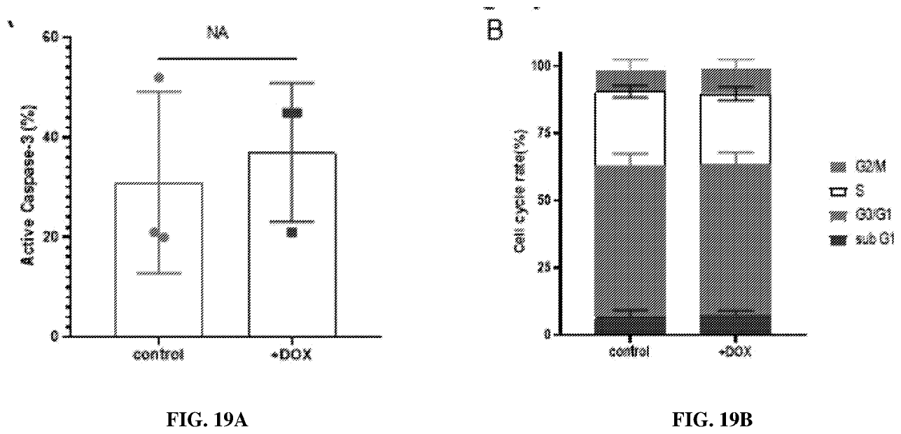

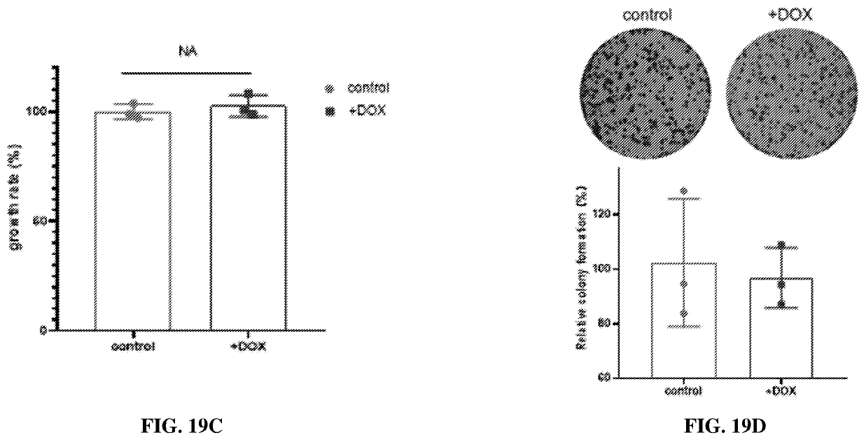

A dCas9-Tet on-p65HSF1-BZLF1 sgRNA3 HeLa cell line was produced. The induction of 3×FLAG-PUFa-p65HSF1 expression by 1 μg/mL Doxycycline (DOX) did not show significant effects on cell proliferation, apoptosis and cell cycle in HeLa cells (FIGS. 19A-19D). This indicates that reactivation of EBV gene expression does not effect EBV-negative cells.

### Materials and Methods

dCas9-Tet on-p65HSF1-BZLF1sgRNA3 cell line was produced as described previously. DOX treatment, FACS and clonogenic assay were conducted as described previously

### Example 13: BRLF1 and BZLF1 gRNAs Trigger EBV Reactivation Synergistically In Vitro

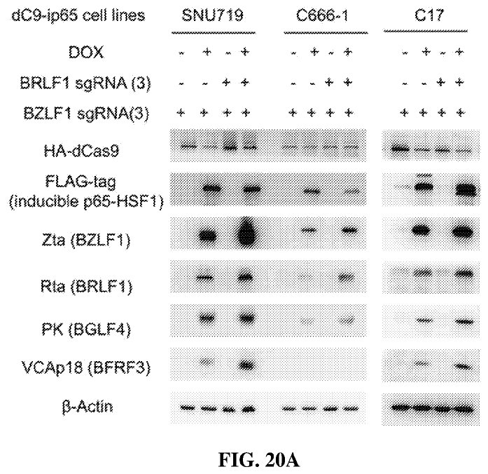

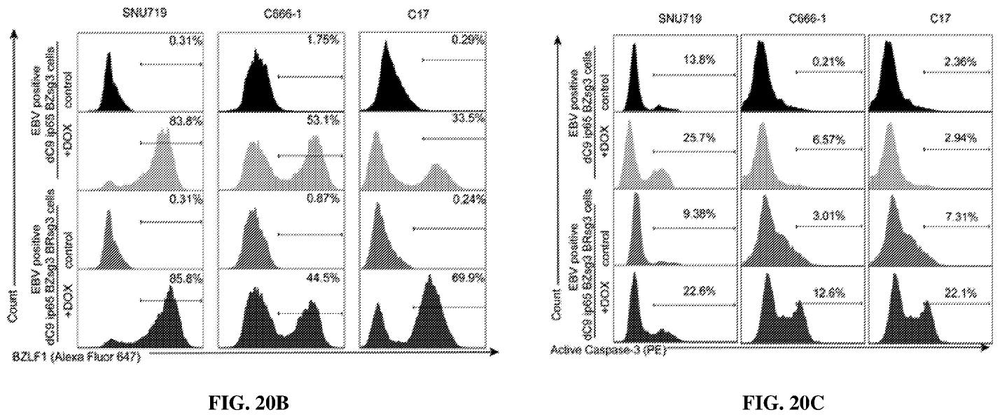

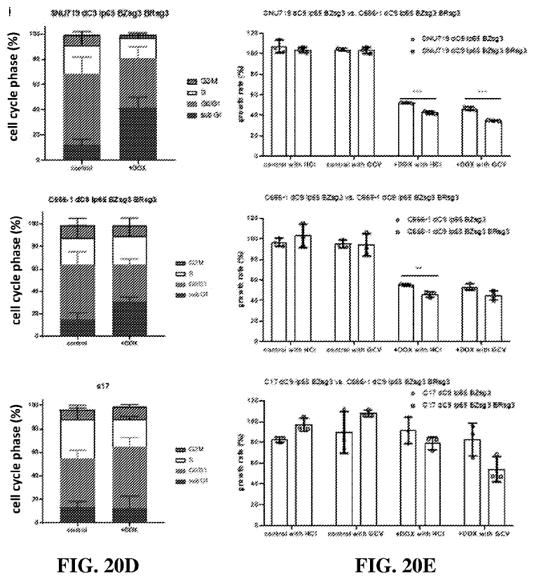

SNU-719, C666-1, and C17 cell lines that express dCas9-Tet on-p65HSF1-BZLF1 sgRNA3-BRLF1 sgRNA3 were constructed, and the expression of EBV lytic proteins BGLF4 (PK) and BFRF3 (VCAp18) were detectable, and synergistic protein production occurred compared to DOX treated dCas9-Tet on-p65HSF1-BZLF1 sgRNA3 SNU-719, C666-1, and C17 cells (FIGS. 20A-20B). The co-expression of BZLF1sgRNA3 and BRLF1 sgRNA3 increased the number of cells expressing active caspase-3, more cells were accumulated in sub G1 phase, and the cell viability was decreased, compared with DOX treated dCas9-Tet on-p65HSF1-BZLF1 sgRNA3 SNU-719, C666-1, and C17 cells (FIGS. 20C-20E).

### Materials and Methods

dCas9-Tet on-p65HSF1-BZLF1 sgRNA3 cell lines were produced as described previously. EBV reactivation, FACS and electrophoretic gel images were conducted as described previously

Producing HA-dCas9-EGFP-Tet-on PUFa-p65HSF1-BZLF1 sgRNA3-BRLF1sgRNA3 cell line. The day prior to transfection, HEK-293FT cells were seeded into a 10 cm culture dish at 70% density. The cells were transfected with the lentiviral packaging plasmids (pRRE (gag/pol), pRSV (rev), and VSVG (envelope)) and a BRLF1 sgRNA3 lentiviral expression plasmid using Lipofectamine 2000 reagent (Invitrogen). At 6 hours post-transfection, medium was exchanged for fresh. At 48 hour post-transfection, 5 mL of medium containing the lentivirus was collected and centrifuged for 10 minutes at 2,000 rpm to remove cellular debris. The supernatant was filtered through a 0.45-micron filter (Millipore), and the lentivirus was collected. SNU-719 dCas9-Tet-on-PUFap65HSF1-BZLF1 sgRNA3 or C666-1 dCas9-Tet-on-PUFa-p65HSF1-BZLF1 sgRNA3, or C17 dCas9-Tet-on-PUFa-p65HSF1-BZLF1sgRNA3 cells were seeded into a 10 cm culture dish at 60% density per dish, transduced with 7 mL of the BRLF1 sgRNA3 lentivirus in culture medium supplemented with 8 μg/mL polybrene for 48 hours, and subsequently selected with G418 antibiotics on the third day post-transduction.

### Example 14: Activation of BGLF4 with BGLF4 sgRNAs

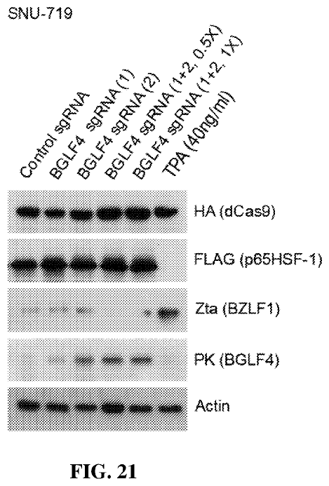

We tested the direct activation of BGLF4 (PK-encoding gene) by single or a pair of sgRNAs targeting the BGLF4 promoter. SNU-719 dCAS9-inducible p65-HSF1 cells were transiently transfected with sgRNAs and cultured in doxycycline-containing media to induce p65-HSF1 expression and assayed for BGLF4 expression by western blot (FIG. 21). The results demonstrated the activation of BGLF4/PK directly independent of its natural activator BZLF1/Zta and that synergistic activation can be achieved by a mixture of promoter-targeting gRNAs.

### Example 15: Activation of BZLF1 by TALE Transactivators

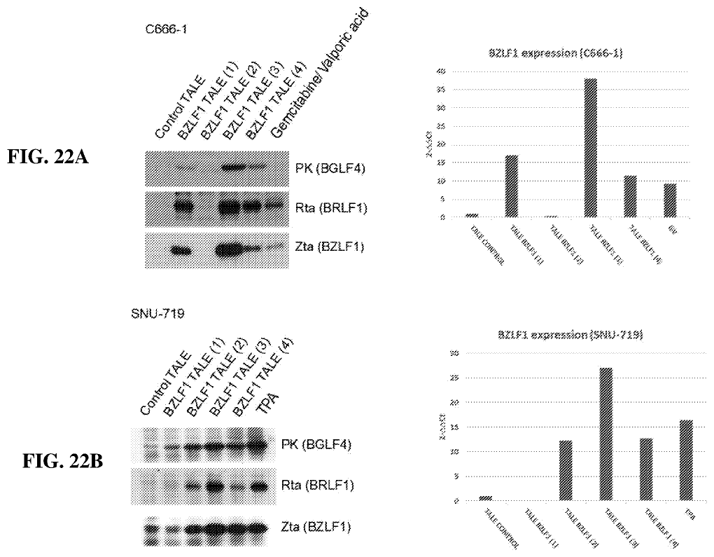

We tested the utility of TALE transactivator for activating BZLF1 expression. Four BZLF1 TALE activators were constructed to target the promoter of BZLF1, and were transiently transfected into EBV-associated cancer cell lines C666-1 (FIG. 22A) and SNU-719 (FIG. 22B). TALE (3) gave the highest activity in both cells, while TALE (2) was only able to activate BZLF1 in C666-1 cells instead of and SNU-719 cells. As expected, activation of BZLF1 results in the activation of BRLF1 and BGLF4.

### Example 16: Activation of BRLF and BGLF4 by TALE Transactivators

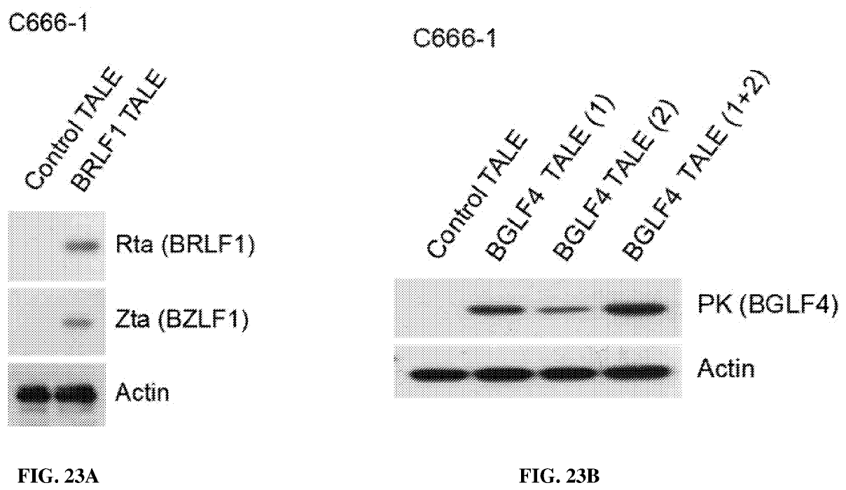

We tested the utility of TALE transactivators for activating BRLF and BGLF4 expression. One TALE transactivator targeting BRLF1 promoter was constructed and transiently transfected into C666-1 cells, resulting in the activation of BRLF1 as well as BZLF1 (FIG. 23A). Two TALE transactivators were constructed and transiently transfected into C666-1 cells, resulting in the activation of BGLF4 expression (FIG. 23B). Combined transfection of both BGLF4 TALE transactivators resulted in the synergistic activation of BGLF4 (FIG. 23B).

SEQUENCES

>SEQ ID NO: 1, amino acid sequence of 3x FLAG-2x NLS-p65HSFl
MDYKDHDGDYKDHDIDYKDDDDKIDGGGGSDPKKKRKVDPKKKRKVDPKKKRKVGS
TGSRNDGGGGSGGGGSGGGGSGRAGILPPKKKRKVSRGRSRLLEDFRNNRYPNLQLREI
AGHIMEFSQDQHGSRFIQLKLERATPAERQLVFNEILQAAYQLMVDVFGNYVIQKFFEF
GSLEQKLALAERIRGHVLSLALQMYGSRVIEKALEFIPSDQQNEMVRELDGHVLKCVKD
QNGNHVVQKCIECVQPQSLQFIIDAFKGQVFALSTHPYGCRVIQRILEHCLPDQTLPILEE
LHQHTEQLVQDQYGNYVIQHVLEHGRPEDKSKIVAEIRGNVLVLSQHKFASNVVEKCV
THASRTERAVLIDEVCTMNDGPHSALYTMMKDQYANYVVQKMIDVAEPGQRKIVMHK
IRPHIATLRKYTYGKHILAKLEKYYMKNGVDLGDPKKKRKVDPKKKRKVGGRGGGGS
GGGGSGGGGSGPAGGGGSGGGGSGGGGSGPKKKRKVAAAGSPSGQISNQALALAPSSA
PVLAQTMVPSSAMVPLAQPPAPAPVLTPGPPQSLSAPVPKSTQAGEGTLSEALLHLQFDA
DEDLGALLGNSTDPGVFTDLASVDNSEFQQLLNQGVSMSHSTAEPMLMEYPEAITRLVT
GSQRPPDPAPTPLGTSGLPNGLSGDEDFSSIADMDFSALLSQISSSGQGGGGSGFSVDTSA
LLDLFSPSVTVPDMSLPDLDSSLASIQELLSPQEPPRPPEAENSSPDSGKQLVHYTAQPLFL
LDPGSVDTGSNDLPVLFELGEGSYFSEGDGFAEDPTISLLTGSEPPKAKDPTVSID*
>SEQ ID NO: 2, amino acid sequence of dCas9-2A-EGFP
MYPYDVPDYASPKKKRKVEASDKKYSIGLAIGTNSVGWAVITDEYKVPSKKFKVLGNT
DRHSIKKNLIGALLFDSGETAEATRLKRTARRRYTRRKNRICYLQEIFSNEMAKVDDSFF
HRLEESFLVEEDKKHERHPIFGNIVDEVAYHEKYPTIYHLRKKLVDSTDKADLRLIYLAL
AHMIKFRGHFLIEGDLNPDNSDVDKLFIQLVQTYNQLFEENPINASGVDAKAILSARLSK
SRRLENLIAQLPGEKKNGLFGNLIALSLGLTPNFKSNFDLAEDAKLQLSKDTYDDDLDNL
LAQIGDQYADLFLAAKNLSDAILLSDILRVNTEITKAPLSASMIKRYDEHHQDLTLLKAL
VRQQLPEKYKEIFFDQSKNGYAGYIDGGASQEEFYKFIKPILEKMDGTEELLVKLNREDL
LRKQRTFDNGSIPHQIHLGELHAILRRQEDFYPFLKDNREKIEKILTFRIPYYVGPLARGNS
RFAWMTRKSEETFTPWNFEEVVDKGASAQSFIERMTNFDKNLPNEKVLPKHSLLYEYFT
VYNELTKVKYVTEGMRKPAFLSGEQKKAIVDLLFKTNRKVTVKQLKEDYFKKIECFDS
VEISGVEDRFNASLGTYHDLLKIIKDKDFLDNEENEDILEDIVLTLTLFEDREMIEERLKT
YAHLFDDKVMKQLKRRRYTGWGRLSRKLINGIRDKQSGKTILDFLKSDGFANRNFMQL
IHDDSLTFKEDIQKAQVSGQGDSLHEHIANLAGSPAIKKGILQTVKVVDELVKVMGRHK
PENIVIEMARENQTTQKGQKNSRERMKRIEEGIKELGSQILKEHPVENTQLQNEKLYLYY
LQNGRDMYVDQELDINRLSDYDVDAIVPQSFLKDDSIDNKVLTRSDKNRGKSDNVPSEE
VVKKMKNYWRQLLNAKLITQRKFDNLTKAERGGLSELDKAGFIKRQLVETRQITKHVA
QILDSRMNTKYDENDKLIREVKVITLKSKLVSDFRKDFQFYKVREINNYHHAHDAYLNA
WGTALIKKYPKLESEFVYGDYKVYDVRKMIAKSEQEIGKATAKYFFYSNIMNFFKTEIT
LANGEIRKRPLIETNGETGEIVWDKGRDFATVRKVLSMPQVNIVKKTEVQTGGFSKESIL
PKRNSDKLIARKKDWDPKKYGGFDSPTVAYSVLVVAKVEKGKSKKLKSVKELLGITIM
ERSSFEKNPIDFLEAKGYKEVKKDLIIKLPKYSLFELENGRKRMLASAGELQKGNELALP
SKYVNFLYLASHYEKLKGSPEDNEQKQLFVEQHKHYLDEIIEQISEFSKRVILADANLDK
VLSAYNKHRDKPIREQAENIIHLFTLTNLGAPAAFKYFDTTIDRKRYTSTKEVLDATLIHQ
SITGLYETRIDLSQLGGDSPKKKRKVEASGSGSGQCTNYALLKLAGDVESNPGPLIKMVS
KGEELFTGVVPILVELDGDVNGHKFSVSGEGEGDATYGKLTLKFICTTGKLPVPWPTLV
TTLTYGVQCFSRYPDHMKQHDFFKSAMPEGYVQERTIFFKDDGNYKTRAEVKFEGDTL
VNRIELKGIDFKEDGNILGHKLEYNYNSHNVYIMADKQKNGIKVNFKIRHNIEDGSVQL
ADHYQQNTPIGDGPVLLPDNHYLSTQSALSKDPNEKRDHMVLLEFVTAAGITLGMDEL
YKGSGATNFSLLKQAGDVEENPGPMAKPLSQEESTLIERATATINSIPISEDYSVASAALS
SDGRIFTGVNVYHFTGGPCAELVVLGTAAAAAAGNLTCIVAIGNENRGILSPCGRCRQV
LLDLHPGIKAIVKDSDGQPTAVGIRELLPSGYVWEG*
>SEQ ID NO 3: amino acid sequence of 3x FLAG-4x NLS-TALE-19-2x NLS-p65HSF1
MDYKDHDGDYKDHDIDYKDDDDKIDGGGGSDPKKKRKVDPKKKRKVDPKKKRKVGS
TGSRNDGGGGSGGGGSGGGGSGRAVDLRTLGYSQQQQEKIKPKVRSTVAQHHEALVG
HGFTHAHIVALSQHPAALGTVAVTYQHIITALPEATHEDIVGVGKQWSGARALEALLTD
AGELRGPPLQLDTGQLVKIAKRGGVTAMEAVHASRNALTGAPLNLTPDQVVAIASNNG
GKQALETVQRLLPVLCQDHGLTPDQVVAIASNIGGKQALETVQRLLPVLCQDHGLTPD
QVVAIASNNGGKQALETVQRLLPVLCQDHGLTPDQVVAIASNIGGKQALETVQRLLPVL
CQDHGLTPDQVVAIASNIGGKQALETVQRLLPVLCQDHGLTPDQVVAIASNIGGKQALE
TVQRLLPVLCQDHGLTPDQVVAIASNGGGKQALETVQRLLPVLCQDHGLTPDQVVAIA
SNGGGKQALETVQRLLPVLCQDHGLTPDQVVAIASNGGGKQALETVQRLLPVLCQDHG
LTPDQVVAIASNNGGKQALETVQRLLPVLCQDHGLTPDQVVAIASNIGGKQALETVQRL
LPVLCQDHGLTPDQVVAIASHDGGKQALETVQRLLPVLCQDHGLTPDQVVAIASNGGG
KQALETVQRLLPVLCQDHGLTPDQVVAIASNNGGKQALETVQRLLPVLCQDHGLTPDQ
VVAIASNNGGKQALETVQRLLPVLCQDHGLTPDQVVAIASHDGGKQALETVQRLLPVL
CQDHGLTPDQVVAIASNIGGKQALETVQRLLPVLCQDHGLTPDQVVAIASNGGGKQAL
ESIVAQLSRPDPALAALTNDHLVALACLGGRPAMDAVKKGLPHAPELIRRVNRRIGERT
SHRVARDPKKKRKVDPKKKRKVGGRGGGGSGGGGSGGGGSGPAGGGGSGGGGSGGG
GSGPKKKRKVAAAGSPSGQISNQALALAPSSAPVLAQTMVPSSAMVPLAQPPAPAPVLT
PGPPQSLSAPVPKSTQAGEGTLSEALLHLQFDADEDLGALLGNSTDPGVFTDLASVDNSE
FQQLLNQGVSMSHSTAEPMLMEYPEAITRLVTGSQRPPDPAPTPLGTSGLPNGLSGDEDF
SSIADMDFSALLSQISSSGQGGGGSGFSVDTSALLDLFSPSVTVPDMSLPDLDSSLASIQEL
LSPQEPPRPPEAENSSPDSGKQLVHYTAQPLFLLDPGSVDTGSNDLPVLFELGEGSYFSEG
DGFAEDPTISLLTGSEPPKAKDPTVSID*
>SEQ ID NO: 4, amino acid sequence of 3xFLAG-4xNLS_TALE-BZLF1pp-1_2xNLS-
p65HSF1, also referred to as BZLF1 TALE (1)
MDYKDHDGDYKDHDIDYKDDDKIDGGGGSDPKKKRKVDPKKKRKVDPKKKRKVGST
GSRNDGGGGSGGGGSGGGGSGRAVDLRTLGYSQQQQEKIKPKVRSTVAQHHEALVGH
GFTHAHIVALSQHPAALGTVAVTYQHIITALPEATHEDIVGVGKQWSGARALEALLTDA
GELRGPPLQLDTGQLVKIAKRGGVTAMEAVHASRNALTGAPLNLTPDQVVAIASNIGG
KQALETVQRLLPVLCQDHGLTPDQVVAIASNNGGKQALETVQRLLPVLCQDHGLTPDQ
VVAIASHDGGKQALETVQRLLPVLCQDHGLTPDQVVAIASNIGGKQALETVQRLLPVLC
QDHGLTPDQVVAIASNIGGKQALETVQRLLPVLCQDHGLTPDQVVAIASNIGGKQALET
VQRLLPVLCQDHGLTPDQVVAIASNNGGKQALETVQRLLPVLCQDHGLTPDQVVAIAS
NNGGKQALETVQRLLPVLCQDHGLTPDQVVAIASNGGGKQALETVQRLLPVLCQDHGL
TPDQVVAIASNNGGKQALETVQRLLPVLCQDHGLTPDQVVAIASNNGGKQALETVQRL
LPVLCQDHGLTPDQVVAIASHDGGKQALETVQRLLPVLCQDHGLTPDQVVAIASHDGG
KQALETVQRLLPVLCQDHGLTPDQVVAIASNNGGKQALETVQRLLPVLCQDHGLTPDQ
VVAIASNNGGKQALESIVAQLSRPDPALAALTNDHLVALACLGGRPAMDAVKKGLPHA
PELIRRVNRRIGERTSHRVARDPKKKRKVDPKKKRKVGGRGGGGSGGGGSGGGGSGPA
GGGGSGGGGSGGGGSGPKKKRKVAAAGSPSGQISNQALALAPSSAPVLAQTMVPSSAM
VPLAQPPAPAPVLTPGPPQSLSAPVPKSTQAGEGTLSEALLHLQFDADEDLGALLGNSTD
PGVFTDLASVDNSEFQQLLNQGVSMSHSTAEPMLMEYPEAITRLVTGSQRPPDPAPTPL
GTSGLPNGLSGDEDFSSIADMDFSALLSQISSSGQGGGGSGFSVDTSALLDLFSPSVTVPD
MSLPDLDSSLASIQELLSPQEPPRPPEAENSSPDSGKQLVHYTAQPLFLLDPGSVDTGSND
LPVLFELGEGSYFSEGDGFAEDPTISLLTGSEPPKAKDPTVSID*
>SEQ ID NO: 5, amino acid sequence of BZLF1 TALE (2)
MDYKDHDGDYKDHDIDYKDDDDKIDGGGGSDPKKKRKVDPKKKRKVDPKKKRKVGS
TGSRNDGGGGSGGGGSGGGGSGRAVDLRTLGYSQQQQEKIKPKVRSTVAQHHEALVG
HGFTHAHIVALSQHPAALGTVAVTYQHIITALPEATHEDIVGVGKQWSGARALEALLTD
AGELRGPPLQLDTGQLVKIAKRGGVTAMEAVHASRNALTGAPLNLTPDQVVAIASHDG
GKQALETVQRLLPVLCQDHGLTPDQVVAIASHDGGKQALETVQRLLPVLCQDHGLTPD
QVVAIASNGGGKQALETVQRLLPVLCQDHGLTPDQVVAIASHDGGKQALETVQRLLPV
LCQDHGLTPDQVVAIASNGGGKQALETVQRLLPVLCQDHGLTPDQVVAIASNNGGKQA
LETVQRLLPVLCQDHGLTPDQVVAIASNGGGKQALETVQRLLPVLCQDHGLTPDQVVA
IASNNGGKQALETVQRLLPVLCQDHGLTPDQVVAIASNIGGKQALETVQRLLPVLCQDH
GLTPDQVVAIASNGGGKQALETVQRLLPVLCQDHGLTPDQVVAIASNNGGKQALETVQ
RLLPVLCQDHGLTPDQVVAIASNGGGKQALETVQRLLPVLCQDHGLTPDQVVAIASHD
GGKQALETVQRLLPVLCQDHGLTPDQVVAIASNIGGKQALETVQRLLPVLCQDHGLTP
DQVVAIASNGGGKQALETVQRLLPVLCQDHGLTPDQVVAIASNNGGKQALETVQRLLP
VLCQDHGLTPDQVVAIASNNGGKQALESIVAQLSRPDPALAALTNDHLVALACLGGRP
AMDAVKKGLPHAPELIRRVNRRIGERTSHRVARDPKKKRKVDPKKKRKVGGRGGGGS
GGGGSGGGGSGPAGGGGSGGGGSGGGGSGPKKKRKVAAAGSPSGQISNQALALAPSSA
PVLAQTMVPSSAMVPLAQPPAPAPVLTPGPPQSLSAPVPKSTQAGEGTLSEALLHLQFDA
DEDLGALLGNSTDPGVFTDLASVDNSEFQQLLNQGVSMSHSTAEPMLMEYPEAITRLVT
GSQRPPDPAPTPLGTSGLPNGLSGDEDFSSIADMDFSALLSQISSSGQGGGGSGFSVDTSA
LLDLFSPSVTVPDMSLPDLDSSLASIQELLSPQEPPRPPEAENSSPDSGKQLVHYTAQPLFL
LDPGSVDTGSNDLPVLFELGEGSYFSEGDGFAEDPTISLLTGSEPPKAKDPTVSID
>SEQ ID NO: 6, amino acid sequence of 3xFLAG-4xNLS_TALE-BZLF1pp-3_2xNLS-
p65HSF1, also referred to as BZLF1 TALE (3)
MDYKDHDGDYKDHDIDYKDDDDKIDGGGGSDPKKKRKVDPKKKRKVDPKKKRKVGS
TGSRNDGGGGSGGGGSGGGGSGRAVDLRTLGYSQQQQEKIKPKVRSTVAQHHEALVG
HGFTHAHIVALSQHPAALGTVAVTYQHIITALPEATHEDIVGVGKQWSGARALEALLTD
AGELRGPPLQLDTGQLVKIAKRGGVTAMEAVHASRNALTGAPLNLTPDQVVAIASNIG
GKQALETVQRLLPVLCQDHGLTPDQVVAIASNGGGKQALETVQRLLPVLCQDHGLTPD
QVVAIASNNGGKQALETVQRLLPVLCQDHGLTPDQVVAIASHDGGKQALETVQRLLPV
LCQDHGLTPDQVVAIASNIGGKQALETVQRLLPVLCQDHGLTPDQVVAIASNGGGKQA
LETVQRLLPVLCQDHGLTPDQVVAIASNNGGKQALETVQRLLPVLCQDHGLTPDQVVA
IASNIGGKQALETVQRLLPVLCQDHGLTPDQVVAIASNNGGKQALETVQRLLPVLCQDH
GLTPDQVVAIASHDGGKQALETVQRLLPVLCQDHGLTPDQVVAIASHDGGKQALETVQ
RLLPVLCQDHGLTPDQVVAIASNIGGKQALETVQRLLPVLCQDHGLTPDQVVAIASHDG
GKQALETVQRLLPVLCQDHGLTPDQVVAIASNIGGKQALETVQRLLPVLCQDHGLTPD
QVVAIASNNGGKQALETVQRLLPVLCQDHGLTPDQVVAIASNNGGKQALESIVAQLSRP
DPALAALTNDHLVALACLGGRPAMDAVKKGLPHAPELIRRVNRRIGERTSHRVARDPK
KKRKVDPKKKRKVGGRGGGGSGGGGSGGGGSGPAGGGGSGGGGSGGGGSGPKKKRK
VAAAGSPSGQISNQALALAPSSAPVLAQTMVPSSAMVPLAQPPAPAPVLTPGPPQSLSAP
VPKSTQAGEGTLSEALLHLQFDADEDLGALLGNSTDPGVFTDLASVDNSEFQQLLNQGV
SMSHSTAEPMLMEYPEAITRLVTGSQRPPDPAPTPLGTSGLPNGLSGDEDFSSIADMDFS
ALLSQISSSGQGGGGSGFSVDTSALLDLFSPSVTVPDMSLPDLDSSLASIQELLSPQEPPRP
PEAENSSPDSGKQLVHYTAQPLFLLDPGSVDTGSNDLPVLFELGEGSYFSEGDGFAEDPT
ISLLTGSEPPKAKDPTVSID*
>SEQ ID NO: 7, amino acid sequence of 3xFLAG-4xNLS_TALE-BZLF1pp-4_2xNLS-
p65HSF1, also referred to as BZLF1 TALE (4)
MDYKDHDGDYKDHDIDYKDDDDKIDGGGGSDPKKKRKVDPKKKRKVDPKKKRKVGS
TGSRNDGGGGSGGGGSGGGGSGRAVDLRTLGYSQQQQEKIKPKVRSTVAQHHEALVG
HGFTHAHIVALSQHPAALGTVAVTYQHIITALPEATHEDIVGVGKQWSGARALEALLTD
AGELRGPPLQLDTGQLVKIAKRGGVTAMEAVHASRNALTGAPLNLTPDQVVAIASNNG
GKQALETVQRLLPVLCQDHGLTPDQVVAIASNGGGKQALETVQRLLPVLCQDHGLTPD
QVVAIASHDGGKQALETVQRLLPVLCQDHGLTPDQVVAIASNGGGKQALETVQRLLPV
LCQDHGLTPDQVVAIASNIGGKQALETVQRLLPVLCQDHGLTPDQVVAIASNIGGKQAL
ETVQRLLPVLCQDHGLTPDQVVAIASNIGGKQALETVQRLLPVLCQDHGLTPDQVVAIA
SNIGGKQALETVQRLLPVLCQDHGLTPDQVVAIASNGGGKQALETVQRLLPVLCQDHG
LTPDQVVAIASNIGGKQALETVQRLLPVLCQDHGLTPDQVVAIASNIGGKQALETVQRL
LPVLCQDHGLTPDQVVAIASNNGGKQALETVQRLLPVLCQDHGLTPDQVVAIASHDGG
KQALETVQRLLPVLCQDHGLTPDQVVAIASNGGGKQALETVQRLLPVLCQDHGLTPDQ
VVAIASNNGGKQALETVQRLLPVLCQDHGLTPDQVVAIASNNGGKQALESIVAQLSRPD
PALAALTNDHLVALACLGGRPAMDAVKKGLPHAPELIRRVNRRIGERTSHRVARDPKK
KRKVDPKKKRKVGGRGGGGSGGGGSGGGGSGPAGGGGSGGGGSGGGGSGPKKKRKV
AAAGSPSGQISNQALALAPSSAPVLAQTMVPSSAMVPLAQPPAPAPVLTPGPPQSLSAPV
PKSTQAGEGTLSEALLHLQFDADEDLGALLGNSTDPGVFTDLASVDNSEFQQLLNQGVS
MSHSTAEPMLMEYPEAITRLVTGSQRPPDPAPTPLGTSGLPNGLSGDEDFSSIADMDFSA
LLSQISSSGQGGGGSGFSVDTSALLDLFSPSVTVPDMSLPDLDSSLASIQELLSPQEPPRPP
EAENSSPDSGKQLVHYTAQPLFLLDPGSVDTGSNDLPVLFELGEGSYFSEGDGFAEDPTI
SLLTGSEPPKAKDPTVSID*
>SEQ ID NO: 32, amino acid sequence of BRLF1 TALE
MDYKDHDGDYKDHDIDYKDDDDKIDGGGGSDPKKKRKVDPKKKRKVDPKKKRKVGS
TGSRNDGGGGSGGGGSGGGGSGRAVDLRTLGYSQQQQEKIKPKVRSTVAQHHEALVG
HGFTHAHIVALSQHPAALGTVAVTYQHIITALPEATHEDIVGVGKQWSGARALEALLTD
AGELRGPPLQLDTGQLVKIAKRGGVTAMEAVHASRNALTGAPLNLTPDQVVAIASHDG
GKQALETVQRLLPVLCQDHGLTPDQVVAIASHDGGKQALETVQRLLPVLCQDHGLTPD
QVVAIASNGGGKQALETVQRLLPVLCQDHGLTPDQVVAIASNHGGKQALETVQRLLPV
LCQDHGLTPDQVVAIASNIGGKQALETVQRLLPVLCQDHGLTPDQVVAIASHDGGKQA
LETVQRLLPVLCQDHGLTPDQVVAIASNIGGKQALETVQRLLPVLCQDHGLTPDQVVAI
ASNHGGKQALETVQRLLPVLCQDHGLTPDQVVAIASHDGGKQALETVQRLLPVLCQDH
GLTPDQVVAIASHDGGKQALETVQRLLPVLCQDHGLTPDQVVAIASHDGGKQALETVQ
RLLPVLCQDHGLTPDQVVAIASNIGGKQALETVQRLLPVLCQDHGLTPDQVVAIASNHG
GKQALETVQRLLPVLCQDHGLTPDQVVAIASNIGGKQALETVQRLLPVLCQDHGLTPD
QVVAIASNHGGKQALETVQRLLPVLCQDHGLTPDQVVAIASNHGGKQALETVQRLLPV
LCQDHGLTPDQVVAIASNHGGKQALETVQRLLPVLCQDHGLTPDQVVAIASNHGGKQA
LESIVAQLSRPDPALAALTNDHLVALACLGGRPAMDAVKKGLPHAPELIRRVNRRIGER
TSHRVARDPKKKRKVDPKKKRKVGGRGGGGSGGGGSGGGGSGPAGGGGSGGGGSGG
GGSGPKKKRKVAAAGSPSGQISNQALALAPSSAPVLAQTMVPSSAMVPLAQPPAPAPVL
TPGPPQSLSAPVPKSTQAGEGTLSEALLHLQFDADEDLGALLGNSTDPGVFTDLASVDNS
EFQQLLNQGVSMSHSTAEPMLMEYPEAITRLVTGSQRPPDPAPTPLGTSGLPNGLSGDED
FSSIADMDFSALLSQISSSGQGGGGSGFSVDTSALLDLFSPSVTVPDMSLPDLDSSLASIQE
LLSPQEPPRPPEAENSSPDSGKQLVHYTAQPLFLLDPGSVDTGSNDLPVLFELGEGSYFSE
GDGFAEDPTISLLTGSEPPKAKDPTVSID*
>SEQ ID NO: 33, amino acid sequence of BGLF4 TALE (1)
MDYKDHDGDYKDHDIDYKDDDDKIDGGGGSDPKKKRKVDPKKKRKVDPKKKRKVGS
TGSRNDGGGGSGGGGSGGGGSGRAVDLRTLGYSQQQQEKIKPKVRSTVAQHHEALVG
HGFTHAHIVALSQHPAALGTVAVTYQHIITALPEATHEDIVGVGKQWSGARALEALLTD
AGELRGPPLQLDTGQLVKIAKRGGVTAMEAVHASRNALTGAPLNLTPDQVVAIASNHG
GKQALETVQRLLPVLCQDHGLTPDQVVAIASNIGGKQALETVQRLLPVLCQDHGLTPD
QVVAIASNGGGKQALETVQRLLPVLCQDHGLTPDQVVAIASNHGGKQALETVQRLLPV
LCQDHGLTPDQVVAIASHDGGKQALETVQRLLPVLCQDHGLTPDQVVAIASNIGGKQA
LETVQRLLPVLCQDHGLTPDQVVAIASNIGGKQALETVQRLLPVLCQDHGLTPDQVVAI
ASNIGGKQALETVQRLLPVLCQDHGLTPDQVVAIASNIGGKQALETVQRLLPVLCQDHG
LTPDQVVAIASNHGGKQALETVQRLLPVLCQDHGLTPDQVVAIASNHGGKQALETVQR
LLPVLCQDHGLTPDQVVAIASNHGGKQALETVQRLLPVLCQDHGLTPDQVVAIASNGG
GKQALETVQRLLPVLCQDHGLTPDQVVAIASNGGGKQALETVQRLLPVLCQDHGLTPD
QVVAIASHDGGKQALETVQRLLPVLCQDHGLTPDQVVAIASHDGGKQALETVQRLLPV
LCQDHGLTPDQVVAIASNGGGKQALETVQRLLPVLCQDHGLTPDQVVAIASNHGGKQA
LESIVAQLSRPDPALAALTNDHLVALACLGGRPAMDAVKKGLPHAPELIRRVNRRIGER
TSHRVARDPKKKRKVDPKKKRKVGGRGGGGSGGGGSGGGGSGPAGGGGSGGGGSGG
GGSGPKKKRKVAAAGSPSGQISNQALALAPSSAPVLAQTMVPSSAMVPLAQPPAPAPVL
TPGPPQSLSAPVPKSTQAGEGTLSEALLHLQFDADEDLGALLGNSTDPGVFTDLASVDNS
EFQQLLNQGVSMSHSTAEPMLMEYPEAITRLVTGSQRPPDPAPTPLGTSGLPNGLSGDED
FSSIADMDFSALLSQISSSGQGGGGSGFSVDTSALLDLFSPSVTVPDMSLPDLDSSLASIQE
LLSPQEPPRPPEAENSSPDSGKQLVHYTAQPLFLLDPGSVDTGSNDLPVLFELGEGSYFSE
GDGFAEDPTISLLTGSEPPKAKDPTVSID*
>SEQ ID NO: 34, amino acid sequence of BGLF4 TALE (2)
MDYKDHDGDYKDHDIDYKDDDDKIDGGGGSDPKKKRKVDPKKKRKVDPKKKRKVGS
TGSRNDGGGGSGGGGSGGGGSGRAVDLRTLGYSQQQQEKIKPKVRSTVAQHHEALVG
HGFTHAHIVALSQHPAALGTVAVTYQHIITALPEATHEDIVGVGKQWSGARALEALLTD
AGELRGPPLQLDTGQLVKIAKRGGVTAMEAVHASRNALTGAPLNLTPDQVVAIASNIG
GKQALETVQRLLPVLCQDHGLTPDQVVAIASHDGGKQALETVQRLLPVLCQDHGLTPD
QVVAIASHDGGKQALETVQRLLPVLCQDHGLTPDQVVAIASNHGGKQALETVQRLLPV
LCQDHGLTPDQVVAIASNIGGKQALETVQRLLPVLCQDHGLTPDQVVAIASNHGGKQA
LETVQRLLPVLCQDHGLTPDQVVAIASNHGGKQALETVQRLLPVLCQDHGLTPDQVVA
IASHDGGKQALETVQRLLPVLCQDHGLTPDQVVAIASNGGGKQALETVQRLLPVLCQD
HGLTPDQVVAIASHDGGKQALETVQRLLPVLCQDHGLTPDQVVAIASNGGGKQALETV
QRLLPVLCQDHGLTPDQVVAIASNGGGKQALETVQRLLPVLCQDHGLTPDQVVAIASNI
GGKQALETVQRLLPVLCQDHGLTPDQVVAIASNHGGKQALETVQRLLPVLCQDHGLTP
DQVVAIASNGGGKQALETVQRLLPVLCQDHGLTPDQVVAIASNGGGKQALETVQRLLP
VLCQDHGLTPDQVVAIASNHGGKQALETVQRLLPVLCQDHGLTPDQVVAIASHDGGKQ
ALESIVAQLSRPDPALAALTNDHLVALACLGGRPAMDAVKKGLPHAPELIRRVNRRIGE
RTSHRVARDPKKKRKVDPKKKRKVGGRGGGGSGGGGSGGGGSGPAGGGGSGGGGSG
GGGSGPKKKRKVAAAGSPSGQISNQALALAPSSAPVLAQTMVPSSAMVPLAQPPAPAPV
LTPGPPQSLSAPVPKSTQAGEGTLSEALLHLQFDADEDLGALLGNSTDPGVFTDLASVD
NSEFQQLLNQGVSMSHSTAEPMLMEYPEAITRLVTGSQRPPDPAPTPLGTSGLPNGLSGD
EDFSSIADMDFSALLSQISSSGQGGGGSGFSVDTSALLDLFSPSVTVPDMSLPDLDSSLASI
QELLSPQEPPRPPEAENSSPDSGKQLVHYTAQPLFLLDPGSVDTGSNDLPVLFELGEGSYF
SEGDGFAEDPTISLLTGSEPPKAKDPTVSID*

TABLE 1
List of gRNA spacer sequences targeting the BZLF1, BRLF1, and BGLF4 genes

| | |
|---|---|
| BZLF1 gRNA spacer sequences | SEQ ID NO: 8, gRNA (A3) |
| | GCAAAGATAGCAAAGGTGGC |
| | SEQ ID NO: 9, gRNA (A4) |
| | GCAGCCTCCTCTGTGATGTCA |
| | SEQ ID NO: 10, gRNA (A5) |
| | GAAACTATGCATGAGCCAC |
| | SEQ ID NO: 11, gRNA (A6) |
| | GCAGAAGTGTCTAAAATAAGC |
| BRLF1 gRNA spacer sequences | SEQ ID NO: 12, gRNA (1) |
| | GAAACACTATCCCGAAGTGG |
| | SEQ ID NO: 13, gRNA (2) |
| | GCATCTACTGAACACCATCG |
| | SEQ ID NO: 14, gRNA (3) |
| | GCACTCCTGACAGCCCAGAGG |
| | SEQ ID NO: 15, gRNA (4) |
| | GTGTACAGCAGCACAAGCTGC |
| | SEQ ID NO: 16, gRNA (5) |
| | GCCCCAAGATCTTAAAGAAGC |
| | SEQ ID NO: 17, gRNA (6) |
| | GCTAAGCTACTACTCCCCCA |
| BGLF4 gRNA spacer sequences | SEQ ID NO: 18, gRNA (1) |
| | GCTGTTTTGCCATTTTATTC |
| | SEQ ID NO: 19, gRNA (2), also referred to as |
| | BGLF4-gRNA (1) target sequence |
| | GCACACACGAGTGATGCAAAA |
| | SEQ ID NO: 20, gRNA (3) |
| | GCAAGGATCATACGTGTCCAC |
| | SEQ ID NO: 21, gRNA (4) |
| | GAGTACGGTAGTTCCAGTGG |
| | SEQ ID NO: 22, gRNA (5), also referred to as |
| | BGLF4-gRNA (2) target sequence |
| | GTACCGAGGCTCTTAGTTGCT |
| | SEQ ID NO: 23, gRNA (6) |
| | GACTGTGTTTCAAACAGAGCG |
| | SEQ ID NO: 24, gRNA (7) |
| | GACTCAAACGTCTCCCTTGCG |

TABLE 2
List of TALE recognition site sequences

| | |
|---|---|
| BZLF1 TALE | SEQ ID NO: 25, TALE (1) |
| recognition site | AGCAAAGGTGGCCGG |
| | SEQ ID NO: 26, TALE (2) |
| | CCTCTGTGATGTCATGG |
| | SEQ ID NO: 27, TALE (3) |
| | ATGCATGAGCCACAGG |
| | SEQ ID NO: 28, TALE (4) |
| | GTCTAAAATAAGCTGG |
| BRLF1 TALE | SEQ ID NO: 29, TALE (1) |
| recognition site | CCTGACAGCCCAGAGGGG |
| BGLF4 TALE | SEQ ID NO: 30, TALE (1) |
| recognition sites | GATGCAAAAGGGTTCCTG |
| | SEQ ID NO: 31, TALE (2) |
| | ACCGAGGCTCTTAGTTGC |

All references, patents and patent applications disclosed herein are incorporated by reference with respect to the subject matter for which each is cited, which in some cases may encompass the entirety of the document.

The indefinite articles “a” and “an,” as used herein the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean±10% of the recited numerical value.

Where a range of values is provided, each value between the upper and lower ends of the range are specifically contemplated and described herein.

What is claimed is:

1. A method for activating a lytic Epstein-Barr virus (EBV) gene, comprising introducing into a cell infected with EBV
a programmable DNA binding protein system that targets a transcriptional regulatory sequence of a lytic EBV gene, and
a transcriptional activator that is linked to a component of the programmable DNA binding protein system and is capable of activating transcription of the lytic EBV gene.

2. The method of claim 1, wherein the transcriptional regulatory sequence is a promoter sequence.

3. The method of claim 1, wherein the lytic EBV gene is an immediate-early viral transactivator gene, a protein kinase gene, a thymidine kinase gene, or is essential for EBV DNA polymerase activity.

4. The method of claim 3, wherein the lytic EBV gene is an immediate-early viral transactivator gene selected from BZLF1 and BRLF1.

5. The method of claim 3, wherein the lytic EBV gene is BGLF4.

6. The method of claim 3, wherein the lytic EBV gene is BXLF1.

7. The method of claim 3, wherein the lytic EBV gene is BMRF1.

8. The method of claim 1, wherein the transcriptional activator comprises or encodes a heat shock factor 1 (HSF1) transactivation domain, optionally p65HSF1.

9. The method of claim 1, wherein the programmable DNA binding protein system comprises a catalytically-inactive RNA-guided engineered nuclease, optionally dCas9, and a guide RNA that binds to the transcriptional regulatory sequence, optionally wherein the lytic EBV gene is selected from BZLF1, BRLF1, BGLF4, BXLF1, and BMRF1.

10. The method of claim 9, wherein the catalytically-inactive RNA-guided engineered nuclease or the guide RNA is linked to the transcriptional activator.

11. The method of claim 9, wherein the programmable DNA binding protein system further comprises a Pumilio-FBF (PUF) domain binding sequence (PBS) linked to the gRNA and a PUF domain that binds to the PBS of the gRNA, and the PUF domain is linked to the transcriptional activator.

12. The method of claim 1, wherein the programmable DNA binding protein system comprises a transcription activator-like effector (TALE) linked to the transcriptional activator, wherein the TALE binds to the transcriptional regulatory sequence, optionally wherein the lytic EBV gene is selected from BZLF1, BRLF1, BGLF4, BXLF1, and BMRF1.

13. The method of claim 1, wherein the programmable DNA binding protein system includes a zinc finger protein (ZFP) linked to the transcriptional activator, wherein the ZFP binds to the transcriptional regulatory sequence, optionally wherein the lytic EBV gene is selected from BZLF1, BRLF1, BGLF4, BXLF1, and BMRF1.

14. The method of claim 1, wherein the transcriptional activator binds to the transcriptional regulatory sequence.

15. The method of claim 1 wherein expression of a component of the programmable DNA binding protein system is inducible, and/or wherein expression of the transcriptional activator is inducible.

16. The method of claim 1, wherein the cell is a mammalian cell and/or a cancer cell.

17. The method of claim 1, further comprising introducing into the cell an antiviral agent, optionally a prodrug.

18. The method of claim 17, wherein the prodrug is selected from ganciclovir, acyclovir, enciclovir, penciclovir, valacyclovir, famciclovir, and bromovinyldeoxyuridine.

19. A method of synergistic Epstein Barr virus (EBV) lytic activation, comprising introducing into a cell infected with EBV (a) a programmable DNA binding protein system that targets a transcriptional regulatory sequence of EBV BZLF1 and a transcriptional regulatory sequence of EBV BRLF1, and (b) a transcriptional activator that is linked to a component of the programmable DNA binding protein system and is capable of activating transcription of the EBV BZLF1 and EBV BRLF1, wherein expression of genes regulated by EBV BZLF1 and EBV BRLF1 is at least 2-fold higher than expression of the same genes resulting from introduction of a programmable DNA binding protein system that targets only EBV BZLF1 or only EBV BRLF1, optionally wherein the genes regulated by EBV BZLF1 and EBV BRLF1 include EBV protein kinase and EBV early antigen diffuse component.

20. A method comprising administering to a subject a programmable DNA binding protein system that targets a transcriptional regulatory sequence of a lytic EBV gene, and a transcriptional activator that is linked to a component of the programmable DNA binding protein system and is capable of activating transcription of the lytic EBV gene, wherein the subject has a cancer associated with EBV-infection.

21. A kit comprising:
programmable DNA binding protein system that targets a transcriptional regulatory sequence of a lytic EBV gene, optionally selected from the group consisting of BZLF1, BRLF1, BGLF4, BXLF1, and BMRF1;
a transcriptional activator, optionally linked to a component of the programmable DNA binding protein system; and
an antiviral agent, optionally ganciclovir (GCV).

22. A cell comprising a programmable DNA binding protein system that targets a transcriptional regulatory sequence of a lytic EBV gene, and a transcriptional activator that is linked to a component of the programmable DNA binding protein system and is capable of activating transcription of the lytic EBV gene.

23. A gRNA linked to a Pumilio-FBF (PUF) domain binding sequence (PBS), wherein the gRNA targets a lytic EBV gene, optionally wherein the PBS is bound to a PUF domain that is linked to a transcriptional activator.

24. A ribonucleoprotein complex comprising a catalytically-inactive RNA-guided engineered nuclease bound to a gRNA that targets a transcriptional regulatory sequence of a lytic EBV gene, wherein the gRNA is linked to a Pumilio-FBF (PUF) domain binding sequence (PBS), and the PBS is bound to a PUF domain that is linked to a transcriptional activator.


SEQUENCE LISTING (37 sequences)

SEQ ID NO: 1 - protein, 822 aa, Artificial Sequence
  Synthetic
MDYKDHDGDYKDHDIDYKDDDDKIDGGGGSDPKKKRKVDPKKKRKVDPKKKRKVGSTGSRNDGGGGSGGGGSGGGGSGRAGILPPKKKRKVSRGRSRLLEDFRNNRYPNLQLREIAGHIMEFSQDQHGSRFIQLKLERATPAERQLVFNEILQAAYQLMVDVFGNYVIQKFFEFGSLEQKLALAERIRGHVLSLALQMYGSRVIEKALEFIPSDQQNEMVRELDGHVLKCVKDQNGNHVVQKCIECVQPQSLQFIIDAFKGQVFALSTHPYGCRVIQRILEHCLPDQTLPILEELHQHTEQLVQDQYGNYVIQHVLEHGRPEDKSKIVAEIRGNVLVLSQHKFASNVVEKCVTHASRTERAVLIDEVCTMNDGPHSALYTMMKDQYANYVVQKMIDVAEPGQRKIVMHKIRPHIATLRKYTYGKHILAKLEKYYMKNGVDLGDPKKKRKVDPKKKRKVGGRGGGGSGGGGSGGGGSGPAGGGGSGGGGSGGGGSGPKKKRKVAAAGSPSGQISNQALALAPSSAPVLAQTMVPSSAMVPLAQPPAPAPVLTPGPPQSLSAPVPKSTQAGEGTLSEALLHLQFDADEDLGALLGNSTDPGVFTDLASVDNSEFQQLLNQGVSMSHSTAEPMLMEYPEAITRLVTGSQRPPDPAPTPLGTSGLPNGLSGDEDFSSIADMDFSALLSQISSSGQGGGGSGFSVDTSALLDLFSPSVTVPDMSLPDLDSSLASIQELLSPQEPPRPPEAENSSPDSGKQLVHYTAQPLFLLDPGSVDTGSNDLPVLFELGEGSYFSEGDGFAEDPTISLLTGSEPPKAKDPTVSID

SEQ ID NO: 2 - protein, 1820 aa, Artificial Sequence
  Synthetic
MYPYDVPDYASPKKKRKVEASDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSPKKKRKVEASGSGSGQCTNYALLKLAGDVESNPGPLIKMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKGSGATNFSLLKQAGDVEENPGPMAKPLSQEESTLIERATATINSIPISEDYSVASAALSSDGRIFTGVNVYHFTGGPCAELVVLGTAAAAAAGNLTCIVAIGNENRGILSPCGRCRQVLLDLHPGIKAIVKDSDGQPTAVGIRELLPSGYVWEG

SEQ ID NO: 3 - protein, 1258 aa, Artificial Sequence
  Synthetic
MDYKDHDGDYKDHDIDYKDDDDKIDGGGGSDPKKKRKVDPKKKRKVDPKKKRKVGSTGSRNDGGGGSGGGGSGGGGSGRAVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASNNGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNGGGKQALESIVAQLSRPDPALAALTNDHLVALACLGGRPAMDAVKKGLPHAPELIRRVNRRIGERTSHRVARDPKKKRKVDPKKKRKVGGRGGGGSGGGGSGGGGSGPAGGGGSGGGGSGGGGSGPKKKRKVAAAGSPSGQISNQALALAPSSAPVLAQTMVPSSAMVPLAQPPAPAPVLTPGPPQSLSAPVPKSTQAGEGTLSEALLHLQFDADEDLGALLGNSTDPGVFTDLASVDNSEFQQLLNQGVSMSHSTAEPMLMEYPEAITRLVTGSQRPPDPAPTPLGTSGLPNGLSGDEDFSSIADMDFSALLSQISSSGQGGGGSGFSVDTSALLDLFSPSVTVPDMSLPDLDSSLASIQELLSPQEPPRPPEAENSSPDSGKQLVHYTAQPLFLLDPGSVDTGSNDLPVLFELGEGSYFSEGDGFAEDPTISLLTGSEPPKAKDPTVSID

SEQ ID NO: 4 - protein, 1155 aa, Artificial Sequence
  Synthetic
MDYKDHDGDYKDHDIDYKDDDKIDGGGGSDPKKKRKVDPKKKRKVDPKKKRKVGSTGSRNDGGGGSGGGGSGGGGSGRAVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASNIGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASNNGGKQALESIVAQLSRPDPALAALTNDHLVALACLGGRPAMDAVKKGLPHAPELIRRVNRRIGERTSHRVARDPKKKRKVDPKKKRKVGGRGGGGSGGGGSGGGGSGPAGGGGSGGGGSGGGGSGPKKKRKVAAAGSPSGQISNQALALAPSSAPVLAQTMVPSSAMVPLAQPPAPAPVLTPGPPQSLSAPVPKSTQAGEGTLSEALLHLQFDADEDLGALLGNSTDPGVFTDLASVDNSEFQQLLNQGVSMSHSTAEPMLMEYPEAITRLVTGSQRPPDPAPTPLGTSGLPNGLSGDEDFSSIADMDFSALLSQISSSGQGGGGSGFSVDTSALLDLFSPSVTVPDMSLPDLDSSLASIQELLSPQEPPRPPEAENSSPDSGKQLVHYTAQPLFLLDPGSVDTGSNDLPVLFELGEGSYFSEGDGFAEDPTISLLTGSEPPKAKDPTVSID

SEQ ID NO: 5 - protein, 1224 aa, Artificial Sequence
  Synthetic
MDYKDHDGDYKDHDIDYKDDDDKIDGGGGSDPKKKRKVDPKKKRKVDPKKKRKVGSTGSRNDGGGGSGGGGSGGGGSGRAVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNNGGKQALESIVAQLSRPDPALAALTNDHLVALACLGGRPAMDAVKKGLPHAPELIRRVNRRIGERTSHRVARDPKKKRKVDPKKKRKVGGRGGGGSGGGGSGGGGSGPAGGGGSGGGGSGGGGSGPKKKRKVAAAGSPSGQISNQALALAPSSAPVLAQTMVPSSAMVPLAQPPAPAPVLTPGPPQSLSAPVPKSTQAGEGTLSEALLHLQFDADEDLGALLGNSTDPGVFTDLASVDNSEFQQLLNQGVSMSHSTAEPMLMEYPEAITRLVTGSQRPPDPAPTPLGTSGLPNGLSGDEDFSSIADMDFSALLSQISSSGQGGGGSGFSVDTSALLDLFSPSVTVPDMSLPDLDSSLASIQELLSPQEPPRPPEAENSSPDSGKQLVHYTAQPLFLLDPGSVDTGSNDLPVLFELGEGSYFSEGDGFAEDPTISLLTGSEPPKAKDPTVSID

SEQ ID NO: 6 - protein, 1190 aa, Artificial Sequence
  Synthetic
MDYKDHDGDYKDHDIDYKDDDDKIDGGGGSDPKKKRKVDPKKKRKVDPKKKRKVGSTGSRNDGGGGSGGGGSGGGGSGRAVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASNIGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNNGGKQALESIVAQLSRPDPALAALTNDHLVALACLGGRPAMDAVKKGLPHAPELIRRVNRRIGERTSHRVARDPKKKRKVDPKKKRKVGGRGGGGSGGGGSGGGGSGPAGGGGSGGGGSGGGGSGPKKKRKVAAAGSPSGQISNQALALAPSSAPVLAQTMVPSSAMVPLAQPPAPAPVLTPGPPQSLSAPVPKSTQAGEGTLSEALLHLQFDADEDLGALLGNSTDPGVFTDLASVDNSEFQQLLNQGVSMSHSTAEPMLMEYPEAITRLVTGSQRPPDPAPTPLGTSGLPNGLSGDEDFSSIADMDFSALLSQISSSGQGGGGSGFSVDTSALLDLFSPSVTVPDMSLPDLDSSLASIQELLSPQEPPRPPEAENSSPDSGKQLVHYTAQPLFLLDPGSVDTGSNDLPVLFELGEGSYFSEGDGFAEDPTISLLTGSEPPKAKDPTVSID

SEQ ID NO: 7 - protein, 1190 aa, Artificial Sequence
  Synthetic
MDYKDHDGDYKDHDIDYKDDDDKIDGGGGSDPKKKRKVDPKKKRKVDPKKKRKVGSTGSRNDGGGGSGGGGSGGGGSGRAVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASNNGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNGGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNNGGKQALESIVAQLSRPDPALAALTNDHLVALACLGGRPAMDAVKKGLPHAPELIRRVNRRIGERTSHRVARDPKKKRKVDPKKKRKVGGRGGGGSGGGGSGGGGSGPAGGGGSGGGGSGGGGSGPKKKRKVAAAGSPSGQISNQALALAPSSAPVLAQTMVPSSAMVPLAQPPAPAPVLTPGPPQSLSAPVPKSTQAGEGTLSEALLHLQFDADEDLGALLGNSTDPGVFTDLASVDNSEFQQLLNQGVSMSHSTAEPMLMEYPEAITRLVTGSQRPPDPAPTPLGTSGLPNGLSGDEDFSSIADMDFSALLSQISSSGQGGGGSGFSVDTSALLDLFSPSVTVPDMSLPDLDSSLASIQELLSPQEPPRPPEAENSSPDSGKQLVHYTAQPLFLLDPGSVDTGSNDLPVLFELGEGSYFSEGDGFAEDPTISLLTGSEPPKAKDPTVSID

SEQ ID NO: 8 - DNA, 20 bp, Artificial Sequence
  Synthetic
gcaaagatagcaaaggtggc

SEQ ID NO: 9 - DNA, 21 bp, Artificial Sequence
  Synthetic
gcagcctcctctgtgatgtca

SEQ ID NO: 10 - DNA, 19 bp, Artificial Sequence
  Synthetic
gaaactatgcatgagccac

SEQ ID NO: 11 - DNA, 21 bp, Artificial Sequence
  Synthetic
gcagaagtgtctaaaataagc

SEQ ID NO: 12 - DNA, 20 bp, Artificial Sequence
  Synthetic
gaaacactatcccgaagtgg

SEQ ID NO: 13 - DNA, 20 bp, Artificial Sequence
  Synthetic
gcatctactgaacaccatcg

SEQ ID NO: 14 - DNA, 21 bp, Artificial Sequence
  Synthetic
gcactcctgacagcccagagg

SEQ ID NO: 15 - DNA, 21 bp, Artificial Sequence
  Synthetic
gtgtacagcagcacaagctgc

SEQ ID NO: 16 - DNA, 21 bp, Artificial Sequence
  Synthetic
gccccaagatcttaaagaagc

SEQ ID NO: 17 - DNA, 20 bp, Artificial Sequence
  Synthetic
gctaagctactactccccca

SEQ ID NO: 18 - DNA, 20 bp, Artificial Sequence
  Synthetic
gctgttttgccattttattc

SEQ ID NO: 19 - DNA, 21 bp, Artificial Sequence
  Synthetic
gcacacacgagtgatgcaaaa

SEQ ID NO: 20 - DNA, 21 bp, Artificial Sequence
  Synthetic
gcaaggatcatacgtgtccac

SEQ ID NO: 21 - DNA, 20 bp, Artificial Sequence
  Synthetic
gagtacggtagttccagtgg

SEQ ID NO: 22 - DNA, 21 bp, Artificial Sequence
  Synthetic
gtaccgaggctcttagttgct

SEQ ID NO: 23 - DNA, 21 bp, Artificial Sequence
  Synthetic
gactgtgtttcaaacagagcg

SEQ ID NO: 24 - DNA, 21 bp, Artificial Sequence
  Synthetic
gactcaaacgtctcccttgcg

SEQ ID NO: 25 - DNA, 15 bp, Artificial Sequence
  Synthetic
agcaaaggtggccgg

SEQ ID NO: 26 - DNA, 17 bp, Artificial Sequence
  Synthetic
cctctgtgatgtcatgg

SEQ ID NO: 27 - DNA, 16 bp, Artificial Sequence
  Synthetic
atgcatgagccacagg

SEQ ID NO: 28 - DNA, 16 bp, Artificial Sequence
  Synthetic
gtctaaaataagctgg

SEQ ID NO: 29 - DNA, 18 bp, Artificial Sequence
  Synthetic
cctgacagcccagagggg

SEQ ID NO: 30 - DNA, 18 bp, Artificial Sequence
  Synthetic
gatgcaaaagggttcctg

SEQ ID NO: 31 - DNA, 18 bp, Artificial Sequence
  Synthetic
accgaggctcttagttgc

SEQ ID NO: 32 - protein, 1258 aa, Artificial Sequence
  Synthetic
MDYKDHDGDYKDHDIDYKDDDDKIDGGGGSDPKKKRKVDPKKKRKVDPKKKRKVGSTGSRNDGGGGSGGGGSGGGGSGRAVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNHGGKQALETVQRLLPVLCQDHGLTPDQVVAIASNIGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNHGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNHGGKQALETVQRLLPVLCQDHGLTPDQVVAIASNIGGKQALETVQRLLPVLCQDHGLTPDQVVAIASNHGGKQALETVQRLLPVLCQDHGLTPDQVVAIASNHGGKQALETVQRLLPVLCQDHGLTPDQVVAIASNHGGKQALETVQRLLPVLCQDHGLTPDQVVAIASNHGGKQALESIVAQLSRPDPALAALTNDHLVALACLGGRPAMDAVKKGLPHAPELIRRVNRRIGERTSHRVARDPKKKRKVDPKKKRKVGGRGGGGSGGGGSGGGGSGPAGGGGSGGGGSGGGGSGPKKKRKVAAAGSPSGQISNQALALAPSSAPVLAQTMVPSSAMVPLAQPPAPAPVLTPGPPQSLSAPVPKSTQAGEGTLSEALLHLQFDADEDLGALLGNSTDPGVFTDLASVDNSEFQQLLNQGVSMSHSTAEPMLMEYPEAITRLVTGSQRPPDPAPTPLGTSGLPNGLSGDEDFSSIADMDFSALLSQISSSGQGGGGSGFSVDTSALLDLFSPSVTVPDMSLPDLDSSLASIQELLSPQEPPRPPEAENSSPDSGKQLVHYTAQPLFLLDPGSVDTGSNDLPVLFELGEGSYFSEGDGFAEDPTISLLTGSEPPKAKDPTVSID

SEQ ID NO: 33 - protein, 1258 aa, Artificial Sequence
  Synthetic
MDYKDHDGDYKDHDIDYKDDDDKIDGGGGSDPKKKRKVDPKKKRKVDPKKKRKVGSTGSRNDGGGGSGGGGSGGGGSGRAVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASNHGGKQALETVQRLLPVLCQDHGLTPDQVVAIASNIGGKQALETVQRLLPVLCQDHGLTPDQVVAIASNGGGKQALETVQRLLPVLCQDHGLTPDQVVAIASNHGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNHGGKQALETVQRLLPVLCQDHGLTPDQVVAIASNHGGKQALETVQRLLPVLCQDHGLTPDQVVAIASNHGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNHGGKQALESIVAQLSRPDPALAALTNDHLVALACLGGRPAMDAVKKGLPHAPELIRRVNRRIGERTSHRVARDPKKKRKVDPKKKRKVGGRGGGGSGGGGSGGGGSGPAGGGGSGGGGSGGGGSGPKKKRKVAAAGSPSGQISNQALALAPSSAPVLAQTMVPSSAMVPLAQPPAPAPVLTPGPPQSLSAPVPKSTQAGEGTLSEALLHLQFDADEDLGALLGNSTDPGVFTDLASVDNSEFQQLLNQGVSMSHSTAEPMLMEYPEAITRLVTGSQRPPDPAPTPLGTSGLPNGLSGDEDFSSIADMDFSALLSQISSSGQGGGGSGFSVDTSALLDLFSPSVTVPDMSLPDLDSSLASIQELLSPQEPPRPPEAENSSPDSGKQLVHYTAQPLFLLDPGSVDTGSNDLPVLFELGEGSYFSEGDGFAEDPTISLLTGSEPPKAKDPTVSID

SEQ ID NO: 34 - protein, 1258 aa, Artificial Sequence
  Synthetic
MDYKDHDGDYKDHDIDYKDDDDKIDGGGGSDPKKKRKVDPKKKRKVDPKKKRKVGSTGSRNDGGGGSGGGGSGGGGSGRAVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASNIGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNHGGKQALETVQRLLPVLCQDHGLTPDQVVAIASNIGGKQALETVQRLLPVLCQDHGLTPDQVVAIASNHGGKQALETVQRLLPVLCQDHGLTPDQVVAIASNHGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNIGGKQALETVQRLLPVLCQDHGLTPDQVVAIASNHGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNHGGKQALETVQRLLPVLCQDHGLTPDQVVAIASHDGGKQALESIVAQLSRPDPALAALTNDHLVALACLGGRPAMDAVKKGLPHAPELIRRVNRRIGERTSHRVARDPKKKRKVDPKKKRKVGGRGGGGSGGGGSGGGGSGPAGGGGSGGGGSGGGGSGPKKKRKVAAAGSPSGQISNQALALAPSSAPVLAQTMVPSSAMVPLAQPPAPAPVLTPGPPQSLSAPVPKSTQAGEGTLSEALLHLQFDADEDLGALLGNSTDPGVFTDLASVDNSEFQQLLNQGVSMSHSTAEPMLMEYPEAITRLVTGSQRPPDPAPTPLGTSGLPNGLSGDEDFSSIADMDFSALLSQISSSGQGGGGSGFSVDTSALLDLFSPSVTVPDMSLPDLDSSLASIQELLSPQEPPRPPEAENSSPDSGKQLVHYTAQPLFLLDPGSVDTGSNDLPVLFELGEGSYFSEGDGFAEDPTISLLTGSEPPKAKDPTVSID

SEQ ID NO: 35 - DNA, 19 bp, Artificial Sequence
  Synthetic
cccaacactccaccacacc

SEQ ID NO: 36 - DNA, 20 bp, Artificial Sequence
  Synthetic
tcttaggagctgtccgaggg

SEQ ID NO: 37 - DNA, 27 bp, Artificial Sequence
  Synthetic
cacacactacacacacccacccgtctc